SEQUENCE LISTING (52 sequences)

SEQ ID NO: 1 - protein, 14 aa, Artificial sequence
  Synthetic construct
  MISC_FEATURE: CDR3 beta TCR2
CAGSQDGMNTEAFF

SEQ ID NO: 2 - protein, 14 aa, Artificial sequence
  Synthetic construct
  MISC_FEATURE: CDR3 beta TCR2
CASSRDGMNTEAFF

SEQ ID NO: 3 - protein, 14 aa, Artificial sequence
  Synthetic construct
  MISC_FEATURE: CDR3 beta TCR2
CAWSPDGMNTEAFF

SEQ ID NO: 4 - protein, 14 aa, Artificial sequence
  Synthetic construct
  MISC_FEATURE: CDR3 beta TCR2
CASSVDGMNTEAFF

SEQ ID NO: 5 - protein, 14 aa, Artificial sequence
  Synthetic construct
  MISC_FEATURE: CDR3 beta TCR2
CASSIDGMNTEAFF

SEQ ID NO: 6 - protein, 14 aa, Artificial sequence
  Synthetic construct
  MISC_FEATURE: CDR3 beta TCR2
CASSGDGMNTEAFF

SEQ ID NO: 7 - protein, 14 aa, Artificial sequence
  Synthetic construct
  MISC_FEATURE: CDR3 beta TCR2
CASSSDGMNTEAFF

SEQ ID NO: 8 - protein, 14 aa, Artificial sequence
  Synthetic construct
  MISC_FEATURE: CDR3 beta TCR2
CASSDDGMNTEAFF

SEQ ID NO: 9 - protein, 14 aa, Artificial sequence
  Synthetic construct
  MISC_FEATURE: CDR3 beta TCR2
CASSPDGMNTEAFF

SEQ ID NO: 10 - protein, 14 aa, Artificial sequence
  Synthetic construct
  MISC_FEATURE: CDR3 beta TCR2
CASSWDGMNTEAFF

SEQ ID NO: 11 - protein, 14 aa, Artificial sequence
  Synthetic construct
  MISC_FEATURE: CDR3 beta TCR2
CASSADGMNTEAFF

SEQ ID NO: 12 - protein, 14 aa, Artificial sequence
  Synthetic construct
  MISC_FEATURE: CDR3 beta TCR2
CAISEDGMNTEAFF

SEQ ID NO: 13 - protein, 14 aa, Artificial sequence
  Synthetic construct
  MISC_FEATURE: CDR3 beta TCR2
CASSEDGMNTEAFF

SEQ ID NO: 14 - protein, 14 aa, Artificial sequence
  Synthetic construct
  MISC_FEATURE: CDR3 beta TCR2
CASSQDGMNTEAFF

SEQ ID NO: 15 - protein, 11 aa, Artificial sequence
  Synthetic construct
  MISC_FEATURE: 1st mimotope
AMGGLLTQLAM

SEQ ID NO: 16 - protein, 9 aa, Artificial sequence
  Synthetic construct
  MISC_FEATURE: 1st mimetope 9mer
AMGGLLTQL

SEQ ID NO: 17 - protein, 9 aa, Artificial sequence
  Synthetic construct
  MISC_FEATURE: TMEM161A 9mer
ALGGLLTPL

SEQ ID NO: 18 - protein, 11 aa, Artificial sequence
  Synthetic construct
  MISC_FEATURE: 2nd mimotope
KLGGLLTMVGV

SEQ ID NO: 19 - protein, 9 aa, Artificial sequence
  Synthetic construct
  MISC_FEATURE: EBV LMP-2A 9mer
CLGGLLTMV

SEQ ID NO: 20 - protein, 11 aa, Artificial sequence
  Synthetic construct
  MISC_FEATURE: EBV LMP-2A 11mer
CLGGLLTMVAG

SEQ ID NO: 21 - protein, 9 aa, Artificial sequence
  Synthetic construct
  MISC_FEATURE: E. coli EntS 9mer
LLGGLLTMV

SEQ ID NO: 22 - protein, 11 aa, Artificial sequence
  Synthetic construct
  MISC_FEATURE: E. coli EntS 11mer
LLGGLLTMVSA

SEQ ID NO: 23 - protein, 9 aa, Artificial sequence
  Synthetic construct
  MISC_FEATURE: Control peptide
GILGFVFTL

SEQ ID NO: 24 - protein, 13 aa, Artificial sequence
  Synthetic construct
  MISC_FEATURE: CDR3-Alpha
CAVLMDSNYQLIW

SEQ ID NO: 25 - protein, 11 aa, Artificial sequence
  Synthetic construct
  MISC_FEATURE: TMEM161A11mer
ALGGLLTPLFL

SEQ ID NO: 26 - protein, 13 aa, Artificial sequence
  Synthetic construct
  MISC_FEATURE: CDR3 beta TCR15
CATRTGGGNTIYF

SEQ ID NO: 27 - protein, 13 aa, Artificial sequence
  Synthetic construct
  MISC_FEATURE: CDR3 beta TCR15
CASRTGPGNTIYF

SEQ ID NO: 28 - protein, 13 aa, Artificial sequence
  Synthetic construct
  MISC_FEATURE: CDR3 beta TCR15
CSARTGTGNTIYF

SEQ ID NO: 29 - protein, 13 aa, Artificial sequence
  Synthetic construct
  MISC_FEATURE: CDR3 beta TCR15
CASRTGDGNTIYF

SEQ ID NO: 30 - protein, 13 aa, Artificial sequence
  Synthetic construct
  MISC_FEATURE: CDR3 beta TCR15
CSARTGVGNTIYF

SEQ ID NO: 31 - protein, 13 aa, Artificial sequence
  Synthetic construct
  MISC_FEATURE: CDR3 beta TCR15
CSVRTGAGNTIYF

SEQ ID NO: 32 - protein, 13 aa, Artificial sequence
  Synthetic construct
  MISC_FEATURE: CDR3 beta TCR15
CAYRTGSGNTIYF

SEQ ID NO: 33 - protein, 13 aa, Artificial sequence
  Synthetic construct
  MISC_FEATURE: CDR3 beta TCR15
CASRTGSGNTIYF

SEQ ID NO: 34 - protein, 11 aa, Artificial sequence
  Synthetic construct
  MISC_FEATURE: Tetraspanin 11mer
AMGGLLFLLGF

SEQ ID NO: 35 - protein, 9 aa, Artificial sequence
  Synthetic construct
  MISC_FEATURE: Tetraspanin 9mer
AMGGLLFLL

SEQ ID NO: 36 - protein, 11 aa, Artificial sequence
  Synthetic construct
  MISC_FEATURE: GRM2 (Glutamate receptor) 11mer
AMGSLLALLAL

SEQ ID NO: 37 - protein, 9 aa, Artificial sequence
  Synthetic construct
  MISC_FEATURE: GRM2 9mer
AMGSLLALL

SEQ ID NO: 38 - protein, 11 aa, Artificial sequence
  Synthetic construct
  MISC_FEATURE: ZNF780B (Zinc finger protein 780B isoform X1) 11mer
KAFGLLTQLAQ

SEQ ID NO: 39 - protein, 9 aa, Artificial sequence
  Synthetic construct
  MISC_FEATURE: ZNF780B 9mer
KAFGLLTQL

SEQ ID NO: 40 - protein, 9 aa, Artificial sequence
  Synthetic construct
  MISC_FEATURE: mnhA1 9mer
KLGGLLTIM

SEQ ID NO: 41 - protein, 9 aa, Artificial sequence
  Synthetic construct
  MISC_FEATURE: NhaB 9mer
ALVGGLLMV

SEQ ID NO: 42 - protein, 9 aa, Artificial sequence
  Synthetic construct
  MISC_FEATURE: PA14 60530 9mer
ALGGVMTMV

SEQ ID NO: 43 - protein, 9 aa, Artificial sequence
  Synthetic construct
  MISC_FEATURE: TMEM161A 9mer
ALGGLLTPL

SEQ ID NO: 44 - protein, 9 aa, Artificial sequence
  Synthetic construct
  MISC_FEATURE: EBV BMLF1 9mer
GLCTLVAML

SEQ ID NO: 45 - protein, 9 aa, Artificial sequence
  Synthetic construct
  MISC_FEATURE: CLDN2 locus
LLGTLVAML

SEQ ID NO: 46 - protein, 9 aa, Artificial sequence
  Synthetic construct
  MISC_FEATURE: SERINC5 locus
YLCTLVAPL

SEQ ID NO: 47 - protein, 9 aa, Artificial sequence
  Synthetic construct
  MISC_FEATURE: TMEM198 locus
HPVGEASIL

SEQ ID NO: 48 - protein, 8 aa, Artificial sequence
  Synthetic construct
  MISC_FEATURE: CDR3 Beta TCR2 motif consensus
  misc_feature (2)..(2): Xaa can be any amino acid
SXDGMNTE

SEQ ID NO: 49 - protein, 7 aa, Artificial sequence
  Synthetic construct
  MISC_FEATURE: CDR3 Beta TCR15 motif consensus
  misc_feature (4)..(4): Xaa can be any amino acid
RTGXGNT

SEQ ID NO: 50 - protein, 7 aa, Artificial sequence
  Synthetic construct
  MISC_FEATURE: CDR3 Beta TCR12 motif consensus
  misc_feature (3)..(3): Xaa can be any amino acid
SVXSNQP

SEQ ID NO: 51 - protein, 7 aa, Artificial sequence
  Synthetic construct
  MISC_FEATURE: CDR3 Beta TCR13 motif consensus
  misc_feature (5)..(5): Xaa can be any amino acid
SIRSXYE

SEQ ID NO: 52 - protein, 7 aa, Artificial sequence
  Synthetic construct
  MISC_FEATURE: CDR3 Beta TCR14 motif consensus
  misc_feature (2)..(2): Xaa can be any amino acid
SXRSTDT

(19) United States
(12) Patent Application Publication
(10) Pub. No.: US 2023/0348559 A1
(43) Pub. Date: Nov. 2, 2023

(54) NOVEL T-CELL SPECIFICITIES AND USES THEREOF

(71) Applicant: The Board of Trustees of the Leland Stanford Junior University, Stanford, CA (US)

(72) Inventors: Diane TSENG, Stanford, CA (US); Mark M. DAVIS, Stanford, CA (US); Crystal L. MACKALL, Palo Alto, CA (US); Shin-Heng CHIOU, Princeton, NJ (US)

(21) Appl. No.: 17/996,966

(22) PCT Filed: Apr. 23, 2021
(86) PCT No.: PCT/US2021/028955

§ 371 Date: Oct. 24, 2022

Related U.S. Application Data
(60) Provisional application No. 63/014,960, filed on Apr. 24, 2020.

(51) Int. Cl. C07K 14/725; C12N 5/0783; C12N 15/86; A61K 39/00; A61P 35/00; A61P 31/00; A61K 45/06

(52) U.S. Cl. CPC C07K 14/7051; C12N 5/0636; C12N 15/86; A61K 39/4611; A61K 39/4632; A61P 35/00; A61P 31/00; A61K 45/06; C12N 2510/00; C12N 2740/10043; C12N 2740/15043; C12N 2501/515; A61K 2239/55

(57) ABSTRACT

The present disclosure relates generally to polypeptide constructs, and particularly relate to T-cell receptor (TCR) constructs having binding affinity for a specific cognate antigen. The disclosure also provides compositions and methods useful for producing such constructs as well as methods for the diagnosis, prevention, and/or treatment of conditions associated with cells expressing the cognate antigen recognized by the polypeptide constructs.

### CROSS-REFERENCE TO RELATED APPLICATIONS

This application claims the benefit of priority to U.S. Provisional Patent Application Ser. No. 63/014,960, filed on Apr. 24, 2020. The disclosure of the above-referenced application is herein expressly incorporated by reference it its entirety, including any drawings.

### STATEMENT REGARDING FEDERALLY SPONSORED R&D

This invention was made with Government support under contract CA232568 awarded by The National Institute of Health. The Government has certain rights in the invention.

### INCORPORATION OF THE SEQUENCE LISTING

The material in the accompanying Sequence Listing is hereby incorporated by reference into this application. The accompanying Sequence Listing text file, named 078430_518001WO_Sequence_Listing.txt, was created on Apr. 20, 2021 and is 15 KB.

### FIELD

The present disclosure relates generally to the field of immunology, and particularly relate to polypeptide constructs having binding affinity for a specific antigen. The disclosure also provides compositions and methods useful for producing such constructs as well as methods for the diagnosis, prevention, and/or treatment of health conditions associated with cells expressing the cognate antigen recognized by the polypeptide constructs.

### BACKGROUND

In recent years, T-cell receptors (TCR) have emerged as a promising approach for immunotherapy and made headlines in clinical trials conducted by a number of pharmaceutical and biotechnology companies. TCRs have been shown to have therapeutic and diagnostic potential and can be modified similarly to antibody molecules. In particular, the affinity of TCRs for a specific antigen makes them valuable for various therapeutic strategies, including adoptive immunotherapy.

In recent years, the wide use of immune checkpoint blockade and T cell-based immunotherapies to treat patients with solid tumors requires a deeper understanding of the T cell specificities in cancer. However, the specificities of the vast majority of tumor-infiltrating T cells remain unknown across all solid tumors despite the availability of advanced technologies for profiling T cell states and repertoires using single-cell sequencing techniques. This is largely due to the absence of tools for analyzing diverse TCR repertoires in the context of highly polymorphic human leukocyte antigens (HLA) alleles. For example, while next-generation sequencing technologies have made the sequencing of large numbers of TCR relatively straightforward and inexpensive, a major problem revolves around how these very large repertoires can be analyzed. This is because there can be hundreds or thousands of possible TCR sequences for the same peptide-MHC specificity.

Accordingly, uncovering the specificities of tumor-infiltrating T cells is important for understanding how T cell-intrinsic factors shape tumor-immune system interactions and impact therapies aimed at harnessing T cell responses against cancer.

### SUMMARY

The present disclosure relates generally to the field of immunology. More particularly, provided herein are novel polypeptide constructs having binding affinity for a specific antigen. The disclosure also provides compositions and methods useful for producing such polypeptide constructs as well as methods for the diagnosis, prevention, and/or treatment of conditions associated with cells expressing the cognate antigen recognized by the polypeptide constructs. In particular, also provided are recombinant cells such as lymphocyte T cells that have been engineered to express a polypeptide construct as disclosed herein and are directed against a cell of interest such as a cancer cell.

In one aspect, provided herein are various constructs including at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-14, 26-33, and 48-49.

Non-limiting exemplary embodiments of the disclosed constructs can include one or more of the following features. In some embodiments, the construct as disclosed herein is capable of binding to an epitope including a sequence having at least 70%, 75%, 80%, 85%, 90%, 95%, 96%, 97%, 98%, 99%, or 100% sequence identity to a sequence selected from the group consisting of SEQ ID NOs: 15-22 and 44-45. In some embodiments, the construct is single-chain constructs or double-chain constructs. In some embodiments, the construct is selected from the group consisting of: (a) a T cell receptor (TCR); (b) an antibody; and (c) a functional derivative or fragment of (a) or (b). In some embodiments, the construct is a TCR construct including a TCR alpha chain and a TCR beta chain covalently linked to each other. In some embodiments, the construct is a TCR construct including in its beta chain a CDR3β having at least 70%, 75%, 80%, 85%, 90%, 95%, 96%, 97%, 98%, 99%, or 100% sequence identity to a sequence selected from SEQ ID NOs: 1-14, 26-33, and 48-49. In some embodiments, the construct further includes in its alpha chain a CDR3α sequence. In some embodiments, the CDR3α sequence has at least 70%, 75%, 80%, 85%, 90%, 95%, 96%, 97%, 98%, 99%, or 100% sequence identity to a sequence selected from SEQ ID NO: 24. In some embodiments, the construct further includes in its alpha chain a CDR3α having at least 70%, 75%, 80%, 85%, 90%, 95%, 96%, 97%, 98%, 99%, or 100% sequence identity to the sequence of SEQ ID NO: 24. In some embodiments, the CDR3α sequence has at least 100% sequence identity to the sequence of SEQ ID NO: 24 and further includes one, two, three, or four amino acid residues of SEQ ID NO: 24 substituted with a different amino acid residue.

In some embodiments, the construct disclosed herein is an antibody construct selected from the group consisting of an antigen-binding fragment (Fab), a single-chain variable fragment (scFv), a nanobody, a single domain antibody (sdAb), a V_{H} domain, a V_{L} domain, a V_{H}H domain, a diabody, or a functional fragment of any thereof.

In another aspect, provided herein are recombinant nucleic acids, wherein the nucleic acids including a nucleic sequence encoding a construct of the disclosure.

Non-limiting exemplary embodiments of the disclosed nucleic acids can include one or more of the following features. In some embodiments, the nucleic acid sequence is operably linked to a heterologous nucleic acid sequence. In some embodiments, the nucleic acid molecule is further defined as an expression cassette or an expression vector. In some embodiments, the vector is a plasmid vector or a viral vector. In some embodiments, the viral vector is derived from a lentivirus, an adeno virus, an adeno-associated virus, a baculovirus, or a retrovirus.

In another aspect, some embodiments of the disclosure relates to engineered cells that include one or more of: (a) a construct of the disclosure and/or (b) a recombinant nucleic acid of the disclosure. Non-limiting exemplary embodiments of the disclosed cells can include one or more of the following features. In some embodiments, the engineered cell is a eukaryotic cell. In some embodiments, the eukaryotic cell is a mammalian cell. In some embodiments, the mammalian cell is a human cell. In some embodiments, the cell is an immune cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune cell is a lymphocyte. In some embodiments, the lymphocyte is a T lymphocyte or a T lymphocyte progenitor. In some embodiments, the T lymphocyte is a CD4+ T cell or a CD8+ T cell. In some embodiments, the T lymphocyte is a CD8+ T cytotoxic lymphocyte cell selected from the group consisting of naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In a related aspect, some embodiments of the disclosure relate to cell cultures that include at least one engineered cell of the disclosure and a culture medium.

In another aspect, some embodiments disclosed herein relate to methods for making an engineered cell, wherein the method includes (a) providing a host cell capable of protein expression; and (b) transducing the provided host cell with a recombinant nucleic acid of the disclosure to produce an engineered cell. Accordingly, in a related aspect, also provided herein are engineered cells produced by the methods of the disclosure. In a further related aspect, some embodiments of the disclosure relate to cell cultures that include at least one engineered cell of the disclosure and a culture medium.

In one aspect, provided herein are various pharmaceutical compositions, wherein the pharmaceutical compositions include a pharmaceutically acceptable carrier and one or more of: (a) a construct of the disclosure; (b) a recombinant nucleic acid of the disclosure; and/or (c) an engineered cell of the disclosure.

Non-limiting exemplary embodiments of the disclosed pharmaceutical compositions can include one or more of the following features. In some embodiments, the composition includes a recombinant nucleic acid of the disclosure and a pharmaceutically acceptable carrier. In some embodiments, the recombinant nucleic acid is encapsulated in a viral capsid or a lipid nanoparticle. In some embodiments, the composition includes an engineered cell of the disclosure and a pharmaceutically acceptable carrier.

In another aspect, some embodiments of the disclosure relate to methods for the prevention and/or treatment of a condition in a subject in need thereof, wherein the methods include administering to the subject a composition including one or more of (a) a construct of the disclosure; (b) a recombinant nucleic acid of the disclosure; (c) an engineered cell of the disclosure; and d) a pharmaceutically composition of the disclosure.

Non-limiting exemplary embodiments of the disclosed methods for preventing and/or treating a condition in a subject in need thereof can include one or more of the following features. In some embodiments, the condition is a proliferative disorder. In some embodiments, the proliferative disorder is a cancer that expresses an epitope including a sequence with at least 70%, 75%, 80%, 85%, 90%, 95%, 96%, 97%, 98%, 99%, or 100% sequence identity to a sequence selected from the group consisting of SEQ ID NOs: 15-22 and 44-45. In some embodiments, the proliferative disorder is a cancer expressing the TMEM161A antigen (TMEM161A-positive cancer). In some embodiments, the TMEM16TA-positive cancer is selected from the group consisting of colon cancer, breast cancer, kidney cancer, lung cancer, ovarian cancer, pancreatic cancer, prostate cancer, sarcoma, neuroendocrine cancer, and testicular cancer. In some embodiments, the proliferative disorder is a cancer expressing the CLDN2 antigen (CLDN2-positive cancer). In some embodiments, the CLDN2-positive cancer is selected from the group consisting of colorectal cancer, cervical cancer, liver cancer, lung cancer, gastric cancer, pancreatic cancer, renal cancer, and stomach cancer. In some embodiments, the lung cancer is selected from the group consisting of adenocarcinoma, squamous cell carcinoma, small cell carcinoma, non-small cell carcinoma, adenosquamous carcinoma, small cell lung cancer, large cell carcinoma, neuroendocrine cancers of the lung, non-small cell lung cancer (NSCLC), undifferentiated non-small cell carcinoma, non-small cell carcinoma not otherwise specified, pulmonary squamous cell carcinoma, broncho-alveolar carcinoma, sarcomatoid carcinoma, pleomorphic carcinoma, carcinosarcoma, pulmonary blastoma, metastatic carcinoma of unknown primary, primary pulmonary lymphoepithelioma-like carcinoma, and benign neoplasms of the lung. In some embodiments, the cancer is a non-metastatic cancer, a metastatic cancer, a multiply drug resistant cancer, or a recurrent cancer. In some embodiments, the administered composition inhibits tumor growth or metastasis of the cancer in the subject.

In some embodiments, provided herein are methods for preventing and/or treating a condition in a subject in need thereof, wherein the condition is a malignancy associated with a bacterial infection or viral infection. In some embodiments, the condition is a malignancy associated with an infection by Epstein-Barr virus (EBV) or Escherichia coli. In some embodiments, the malignancy is associated with an EBV infection and is selected from the group consisting of Hodgkin lymphoma, Burkitt lymphoma, diffuse large B cell lymphoma, nasopharyngeal carcinoma, gastric carcinoma, post-transplant lymphoproliferative disease, B lymphoproliferative disease, T/NK lymphoproliferative disease, T/NK lymphomas/leukemias, leiomyosarcomas, and lymphoepithelioma-like carcinomas.

In some embodiments of the methods for preventing and/or treating a condition in a subject in need thereof, wherein the subject is a mammal. In some embodiments, the mammal is a human. In some embodiments, the composition is administered to the subject individually as a first therapy (monotherapy) or in combination with at least one additional therapies. In some embodiments, the at least one additional therapies is selected from the group consisting of chemotherapy, radiotherapy, immunotherapy, hormonal therapy, toxin therapy, targeted therapy, or surgery. In some embodiments, the first therapy and the at least one additional therapies are administered concomitantly. In some embodiments, the first therapy is administered at the same time as the at least one additional therapies. In some embodiments, the first therapy and the at least one additional therapies are administered sequentially. In some embodiments, the first therapy is administered before the at least one additional therapies. In some embodiments, the first therapy is administered after the at least one additional therapies. In some embodiments, the first therapy is administered before and/or after the at least one additional therapies. In some embodiments, the first therapy and the at least one additional therapies are administered in rotation. In some embodiments, the first therapy and the at least one additional therapies are administered together in a single formulation.

In another aspect, some embodiments of the disclosure relate to kits for the practice of the methods disclosed herein. Some embodiments relate to kits for methods of the diagnosis, prevention, and/or treatment a condition in a subject in need thereof, wherein the kits include one or more of: a construct of the disclosure; a recombinant nucleic acid of the disclosure; an engineered cell of the disclosure; and a pharmaceutical composition of the disclosure.

In another aspect, provided herein is the use of one or more of: a construct of the disclosure; a recombinant nucleic acid of the disclosure; an engineered cell of the disclosure; and a pharmaceutical composition, for the prevention and/or treatment of a condition. In some embodiments, the condition is a proliferative disorder. In some embodiments, the proliferative disorder is a cancer. In some embodiments, the condition is a malignancy associated with an infection. In some embodiments, the infection is a bacterial infection or viral infection.

In another aspect, provided herein is the use of one or more of: a construct of the disclosure, a recombinant nucleic acid of the disclosure, an engineered cell of the disclosure, or a pharmaceutical composition of the disclosure, in the manufacture of a medicament for the treatment of a health condition. In some embodiments, the condition is a proliferative disorder. In some embodiments, the proliferative disorder is a cancer. In some embodiments, the condition is a malignancy associated with an infection. In some embodiments, the infection is a bacterial infection or viral infection.

In yet another aspects, provided herein are various methods for obtaining a construct as disclosed herein, the methods include (a) identifying a plurality of T cell receptors (TCRs) associated with a health condition; (b) determining a sequence of a CDR3β present in each of the identified TCRs; (c) identifying one or more cognate antigens commonly recognized by the CDR3β sequences; (c) making a construct including a CDR3β sequence determined in (b), wherein the construct is capable of binding to the one or more cognate antigens. In some embodiments, the condition is a proliferative disease.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1G schematically summarize the results of experiments performed to establish specificity groups with TCR CDR3β sequences from lung cancer patients. FIG. 1A: Schematic of the three steps involved in TCR specificity inference with the GLIPH2 algorithm (Grouping of Lymphocyte Interactions by Paratope Hotspots). Step 1, acquisition of T cell receptor CDR3β sequences. Step 2, discovery of short sequence motifs within CDR3β sequences from multiple patients. These shared motifs are predicted to be involved in the direct engagement with antigenic peptides loaded on HLA molecules. Step 3, specificity group inference. Multiple cutoffs are used for the inference of a given specificity group, including a) the enrichment of Vβ genes, b) minimum numbers of distinct CDR3β sequences=3, c) minimum numbers of patients=2, d) enrichment of clonally expanded CDR3β clonotypes, and e) enrichment of HLA alleles. FIG. 1B: Disease relevance of tumor-enriched TCR specificity groups in lung cancer. The most expanded CDR3β sequences from the tumors of TRACERx NSCLC patients (validation) belonged to the 449 tumor-enriched specificity groups defined with the MD Anderson cohort (discovery), whereas those from the lungs of healthy and COPD patients did not. The T cell repertoires were obtained from the lungs of two independent, non-cancerous cohorts (healthy and COPD lungs) and tumors of a second NSCLC cohort (the TRACERx consortium, n=202, validation). ***, p<0.001; *, p<0.05 by paired t test. FIG. 1C: Network analysis of 396 NSCLC specificity groups annotated with publicly available, tetramer-derived CDR3β sequences of defined specificities and HLA restrictions. Groups are annotated with tetramer-derived CDR3β sequences from influenza virus (Flu, red), Epstein-Barr virus (EBV, green), and cytomegalovirus (CMV, blue) antigens. Each dot is a specificity group, edges indicate the presence of identical CDR30 sequence(S) across two specificity groups. FIG. 1D: Percentage (%) of HLA-A*02 or HLA-B*08 tetramer-annotated specificity groups with significantly enriched HLA I alleles of the A*02 (purple, right plot) or B*08 (blue, left plot) supertype quantified by GLIPH2, respectively. Specificity groups annotated with tetramers of other HLA alleles (other tetramer) were included for comparisons. FIG. 1E: Percentage (%) of shared specificities between any two given NSCLC patients (n=178) based on total specificity groups regardless of clonal expansion (n=66,996), clonally-expanded specificity groups (n=4,300), or identical CDR3β sequences. FIGS. 1F-1G: Bootstrapping of specificity group numbers (y-axis, specificity group #) with varying sampling sizes for either HLA-A*02+ or HLA-A*02-NSCLC patients (FIG. 1F) or healthy donors (FIG. 1G, Emerson study)

FIGS. 2A-2C schematically summarize the results of experiments performed to illustrate the prioritization of the tumor-enriched specificity group with the motif “S % DGMNTE” in human lung cancer, where “%” denotes the amino acid that varied (Gee M. H., et al., Cell, 2018. 172(3): p. 549-563 e16). FIG. 2A: Left, volcano plot showing the comparison of the 4,300 clonally-expanded TCR specificity groups between tumor (T) and uninvolved lung (N) by Poisson test. The y-axis represents the negative log 10 converted p values of the Poisson test and the x-axis represents the log 2 converted fold-difference between tumor and uninvolved lung (T/N). The dot size represents levels of clonal expansion. Tumor-enriched specificity groups (n=449) are highlighted in red. Right, volcano plot of T/N comparison for CDR3β-defined clonotypes. CDR3β clonotypes belonging to the 449 tumor-enriched specificity groups are highlighted in red. FIG. 2B: Left, volcano plot for the 4,300 NSCLC specificity groups as in (FIG. 2A, left). The specificity groups significantly enriched with the HLA-A*02 allele are highlighted in red or pink. *, specificity groups with at least one pair of CDR3α/β sequences from the Stanford NSCLC cohort are in red. Right, volcano plot of T/N comparison for CDR3β-defined clonotypes as in (A, right). CDR3β clonotypes belonging to the TCR specificity group with the motif “S % DGMNTE” are highlighted in red. FIG. 2C: The CDR3β clonotypes belonging to specificity group with the motif “S % DGMNTE”. Fourteen distinct CDR3β-defined clonotypes were identified in this specificity group. For each of these clonotypes, the Vβ gene usage (Vβ), number of patients with each clonotype in tumor or uninvolved lung (Patient counts), number of HLA-A*02+ patients (Counts of HLA-A*02+ cases/total), and the average clonal frequencies found in uninvolved lung and tumor are shown. ND, not detected.

FIGS. 3A-3D schematically summarize the results of experiments illustrating the identification of tumor and pathogen-derived antigens recognized by a tumor-enriched TCR in human lung cancer. FIG. 3A: Top: levels of stimulation (CD69 upregulation by fold-change compared to unstimulated control) by the indicated top-20 11 mers on Jurkat cells expressing the prioritized TCRα/β chains (TCR2); bottom: ranked raw counts (log 10) of the enriched 11 mer sequences from the 4th-round of selection on the yeast 11 mer library. FIG. 3B: Protein database search results show sequence similarity of the top 2 mimotopes with 9mer peptide sequences from human TMEM161A locus, EBV LMP-2A, and E. coli EntS. All matches were 9mers and predicted to bind HLA-A*02 with high affinities by netMHCpan 4.0. FIG. 3C: Left, representative FACS plots showing the stimulation of the TCR2-expressing Jurkat cells with 9mer from the human TMEM161A locus (TMEM9mer), LMP-2A of EBV (LMP9mer), and EntS from E. coli (EntS9mer); right, results of TCR2-expressing Jurkat cell stimulation as left in triplicate. Ctrl pp: control peptide (GILGFVFTL; SEQ ID NO: 20); No pp: no peptide. FIG. 3D: Stimulation of primary T cells ectopically expressing TCR2 TCR_{α/β} chains with either peptide 9mers (left) or the full-length proteins TMEM161A, EntS, or LMP2A processed by 293T cells and presented on HLA-A*02 (right). Stimulation of primary T cells ectopically expressing TCR14 by 293T-A*02 cells expressing full-length FluM1 protein. *, p<0.05; **, p<0.01; ***, p<0.001 by t test.

FIGS. 4A-4E schematically summarize the results of experiments demonstrating that TMEM161A protein is highly expressed in human lung cancer. FIG. 4A: Representative images of TMEM161A immunohistochemistry on tumor (top) and uninvolved lung (bottom) from 4 Stanford patients. Scale bar, 100 μm. Right-most panel, zoomed in images from the patient A16 tumor section with TMEM161A immunohistochemistry (top) and H&E staining on a serial section (bottom) are shown. Scale bar, 40 μm. FIG. 4B: Quantification of TMEM161A immunohistochemistry on sections from the Stanford NSCLC cohort (n=11). TMEM161A expression on sections was quantified with FIJI. Boxplots represent medians with the first and third quartiles (the 25th and 75th percentiles). Individual data points are included. FIG. 4C: TMEM161A expression quantified by bulk RNA-sequencing for the indicated samples from TCGA cohort (n=958) is shown in boxplots as in FIG. 4B. UI-Ctrl, uninvolved lung control. TMEM161A expression normalized against UI-Ctrl is shown. FIG. 4D: Geneset enrichment analysis (GSEA) for the ranked gene list based on Pearson correlation with TMEM161A abundance in the pan-lung cancer TCGA dataset (n=958). Left, genesets with highest (blue) and lowest (red) normalized enrichment scores based on Pearson correlation with TMEM161A abundance are indicated and their enrichment curves are shown (right). FIG. 4E: Two of the three most enriched genesets in FIG. 4D are chosen and the single-sample GSEA signature scores (Sig score) for the chosen genesets are plotted against TMEM161A expression. Pearson correlation coefficients are shown in plots (cor coef). ***, p<0.001. ND, not significantly different.

FIGS. 5A-5E schematically summarize the results of experiments illustrating the isolation and characterization of cross-reactive TMEM161A-specific T cells from peripheral blood of healthy donors. FIG. 5A: Schematic showing the procedure used to identify the TMEM161A-specific or EntS-specific T cell clones from healthy HLA-A*02+ donors and NSCLC patients. Cells were sorted by FACS directly into 96-well plates for single-cell RNA-seq and TCR-seq. FIG. 5B: Representative FACS plots of T cells sorted with HLA-A*02 tetramers loaded with TMEM9mer (ALGGLLTPL, SEQ ID NO: 17, top panels) or EntS9mer (LLGGLLTMV, SEQ ID NO: 21; bottom panels) from the PBMC of HLA-A*02+ healthy donors (He65 and He66) or NSCLC patients (A6 and A17) are shown. FIG. 5C: Percentages of A*02-tetramer+ T cells as in (B) from healthy donors (n=11) and NSCLC patients (n=7) are summarized in the barplot with individual data points. Boxes represent medians with the first and third quartiles (the 25th and 75th percentiles). No statistical significance was found between the groups. FIG. 5D: Percentages of distinct CDR3β sequences in tetramer-sorted T cells as in FIGS. 5B and 5C from healthy donors and NSCLC patient are shown. Numbers in bars represent the counts of sorted cells. FIG. 5E: Indicated T cell clonotypes identified with tetramer sorting as in FIG. 5B were subcloned into Jurkat cells and subsequently stimulated with the indicated 9mer peptides. Y axis (fold stimulated) shows CD69 upregulation by fold-change compared to unstimulated control. *, p<0.05.

FIGS. 6A-6F schematically summarize the results of experiments illustrating the phenotypic characterization of TMEM161A-specific T cells in tumors. FIGS. 6A-6B: Dimension reduction by Uniform Manifold Approximation and Projection (UMAP) of the single-cell RNA-sequencing (scRNA-Seq) results from 2,950 CD3+ sorted, tumor-infiltrating T cells integrated from resected tumors of 10 NSCLC patients (Stanford cohort). The identified clusters (n=14) of cells with shared cellular states are labeled with distinct colors (FIG. 6A) and shown with varying dot sizes representing the level of clonal expansion (FIG. 6B). FIG. 6C: Level of clonal expansion for the 2,950 sorted T cells as in FIG. 6B is quantified as clonality (1−Pielou's evenness, Methods). FIG. 6D: Breakdown of scRNA-Seq-defined cell states for T cell clonotypes with CDR3β sequences related to the 4,300 clonally-expanded specificity groups (top), inferred viral-related specificity groups annotated with public tetramer datasets (second from top), the 449 tumor-enriched specificity groups (third from top), and specific CD8+ T cells sorted with the HLA-A*02/TMEM9mer tetramer from a tumor (bottom). FIG. 6E: Heatmap showing specific gene expression programs unique to each cell cluster defined in (A). Select differential genes for cluster C5, C6, and C7 are highlighted. FIG. 6F: Stacked violin plot showing the expression of indicated differential genes of C5, C6, and C7 as in FIG. 6E in all cell clusters.

FIGS. 7A-7B schematically summarize the data availability for the 178 HLA-typed NSCLC patients from the MD Anderson Cancer Center (FIG. 7A), and the specificity inference pipeline (FIG. 7B).

FIG. 8 depicts the grouping of low percentages of TCR clonotypes from uninvolved lungs into tumor-enriched specificity groups. The percentages of the top 20 most expanded CDR3β clonotypes from the uninvolved lungs of patients belonging to the MD Anderson NSCLC cohort (n=178, left) and the TRACERx cohort (n=63, right) were quantified for those that belonged to the 449 tumor-enriched specificity groups as in FIG. 1B (% tumor-enriched, log 10-converted). The same analysis was performed on the remainder of the CDR3β clonotypes (Non-exp, non-expanded). ND, no statistical significance was found between the expanded and the less expanded clonotypes.

FIGS. 9A-9B schematically summarize the in silico validation of TCR specificity groups using HLA tetramer sequences. FIG. 9A: Left to right, network analysis of 72 clonally expanded specificity groups colored as in FIG. 1C is shown; the two Flu-related communities (red) are circled and the CDR3β members of the specificity groups are shown with the previously reported short motifs “RS” and “GxY” highlighted in red; heatmap showing distinct CDR3β members (columns) of the Flu-related (with the “RS” motif) specificity groups (rows) and the levels of shared CDR3β members between specificity groups within the circled community; table showing an example of the “SIRSS % E” specificity group containing the short “RS” motif (bold) that is annotated with 5 Flu-specific tetramer sequences (bottom). The counts of distinct CDR3β members from tumor and the VP gene usage are shown (top). FIG. 9B: the 72 clonally expanded specificity groups annotated and colored with the indicated tetramers are shown in the network.

FIGS. 10A-10B schematically summarize the results of CDR3β sequences recognizing CMV, Flu, and EBV do not differ in their distribution between tumor and uninvolved lung. FIG. 10A: Volcano plots showing the relative distributions of CDR3β sequences with inferred specificities to CMV (blue), Flu (red), or EBV (green) across the tumor (T) and uninvolved lung (N) by comparing multiple patients with Poisson test. The y-axis shows the negative log 10 converted p values of the Poisson test and the x-axis shows the log 2 converted fold-difference between tumor and uninvolved lung (T/N). FIG. 10B: Total frequencies of clonotypes in tumor (right) or uninvolved lung (left) that are inferred to recognize antigens from EBV, CMV, or Flu by GLIPH2. Each dot is a patient and total frequencies are shown as log 10 converted values (first and the third quartiles show 25th & 75th percentiles, respectively).

FIGS. 11A-11C schematically summarize the results of GLIPH2 analysis illustrating that TCR specificity group saturation is dependent of the level of clonal expansion, the absolute numbers of specificity groups, as well as the sequencing depth of the repertoires. FIG. 11A: Bootstrapping for quantification of clonally expanded (right, n=77) and non-expanded (left, n=77) HLA-A*02:01-enriched specificity groups with CDR30 sequences from either HLA-A02+(red) or HLA-A02- (blue) NSCLC patients. Bootstrapping was done by “sampling with replacement” and the X axis represents the number of patients that were randomly sampled (Sampling size, Methods) and the Y axis represents the numbers of specificity groups quantified with a given sampling event. Shades of error bars represent the 3× standard errors derived from 100 sampling events for a given sampling size. FIG. 11B: Bootstrapping for quantification of HLA-A*02:01-enriched specificity groups with varying cutoffs for HLA-A*02:01 enrichment (p<0.05, n=1267; p<0.025, n=319; p<0.01, n=72). As in FIG. 11A, X axis represents the number of patients randomly sampled (Sampling size, Methods). Y axis represents the numbers of specificity groups quantified with a given sampling event and normalized against the number of total specificity groups used (Specificity fraction). Shades of error bars represent the 3×standard errors derived from 100 sampling events for a given sampling size. FIG. 11C: Bootstrapping for quantification of HLA-A*02:01-enriched specificity groups with varying input CDR3β sequencing depth (50, 75, 87.5, or 100% of total input by random down-sampling). As in FIG. 11B, X axis represents the number of patients randomly sampled and Y axis represents the normalized numbers of specificity groups. Shades of error bars represent the 3×standard errors derived from 100 sampling events for a given sampling size.

FIG. 12 is a schematic of the combined single-cell TCR-Seq and single-cell RNA-Seq (scRNA-seq) procedures. CD45+CD3+ T cells were sorted from single-cell suspensions of lung tumor samples from patients with NSCLC at Stanford. Single-cell TCR-Seq was performed using nested multiplexed PCR as previously described (Han et al., 2014. Nat. Biotechnol. 32, 684]. Single-cell RNseq was performed according to previous methods (Picelli et al., 2014. Nat. Protoc. 9, 171) with modifications as details in the methods. TCR repertoires were integrated from the single-cell TCR-Seq pipeline and from the scRNA-seq data with reconstruction using the TraCeR algorithm (Stubbington et al., 2016. Nat. Methods 13, 329) for GLIPH2 analysis.

FIGS. 13A-13B depict an experimental validation of four TCR specificity groups inferred by GLIPH2 to recognize Flu and EBV. FIG. 13A: GLIPH2 inferred clone TCR12, TCR13 and TCR14 to recognize Influenza virus M1 (FluM1) 9mer peptide “GILGFVFTL” (SEQ ID NO: 23) in the context of HLA-A*02; clone TCR15 is inferred to recognize EBV BMLF1 9mer peptide “GLCTLVAML” (SEQ ID NO: 44) in the context of HLA-A*02. The CDR3β sequences of the TCR12, TCR13, TCR14, and TCR15 clonotypes belong to specificity groups with the motif “SV % SNQP” (SEQ ID NO: 50), “SIRS % YE” (SEQ ID NO: 51), “S % RSTDT” (SEQ ID NO: 52) and “RTG % GNT” (SEQ ID NO: 49), respectively, where “%” denotes the amino acid that varied (Gee M. H., et al., 2018 supra). The selected T cell clones with paired CDR3α/β sequences available are highlighted in bold in the tables and the CDR3 sequences of both TCRα/β chains are shown at the bottom. All four clones were found in tumors from 2 different NSCLC patients from the Stanford cohort. FIG. 13B: Right, the TCRα/β sequences of the four chosen T cell clonotypes in FIG. 13A were ectopically expressed in Jurkat cells and stimulated with T2 (HLA-A02+) cells pulsed with indicated peptides (right, above FACS plots). CD69 expression quantified by FACS is shown. Left, Jurkat cells expressing the control TCRα/β chains (Ctrl-TCR, PDB 5euo) previously reported to recognize FluM1 in the context of HLA-A*02 and stimulated with or without the 9mer peptide “GILGFVFTL” (SEQ ID NO: 23) are shown. Ctrl PP, control peptide.

FIGS. 14A-14B schematically summarize the results of experiments showing that the antigens recognized by TCR2 are defacto 9mers. FIG. 14A: TCR-deficient Jurkat cells were transduced with lentivirus carrying a composite coding region of TCR2a chain, 2A peptide sequence (2Ap), TCR2β chain, 2Ap, GFP. Transduced cells were subsequently sorted by FACS for GFP+CD3+ population (left) and allowed to expand (top right) and used in in vitro stimulation experiments (bottom right). Similar strategies were used to make other stable TCR-Jurkat clones throughout the paper. FIG. 14B: Jurkat cell clones stably expressing the prioritized TCR2 α/β chains was stimulated with T2 (HLA-A2+) cells pulsed with indicated peptides, including the top mimetope from the yeast screen (11 mer, AMGGLLTQLAM; SEQ ID NO: 15), the predicted 9mer from the top mimetope that can bind HLA-A*02 with high affinity (AMGGLLTQL; SEQ ID NO: 16), and both 9mer/11 mer peptides from the TMEM161A coding region (ALGGLLTPL, SEQ ID NO: 17 and ALGGLLTPLFL; SEQ ID NO: 25, respectively) with sequence homology to the top mimetope. CD69 upregulation was quantified by FACS. Jurkat cells expressing the control TCR_{α/β} chains (TCR-fluM1, PDB 5euo) and stimulated with the cognate 9mer “GILGFVFTL” (SEQ ID NO: 23), the control peptide (Ctrl PP), no peptide (No PP), or no co-cultured T2 cells are included as controls.

FIGS. 15A-15C schematically summarize the results of experiments demonstrating that the endogenous peptide derived from human TMEM161A is recognized by TCR2. FIG. 15A: Protein database search results show partial matches of the top 2 mimotopes with the candidate coding sequences. All matches were 9mers and predicted to bind HLA-A*02 with high affinities by netMHCpan 4.0. FIGS. 15B and 15C: Similarly to FIG. 3C, Jurkat cells expressing TCR2 were cocultured with HLA-A02+ T2 cells and pulsed with indicated peptides: Ctrl PP, GILGFVFTL (SEQ ID NO: 23); top mimetope 11 mer, AMGGLLTQLAM (SEQ ID NO: 15); TMEM161A 11 mer, ALGGLLTPLFL (SEQ ID NO: 25); TMEM161A 9mer, ALGGLLTPL; Tetraspanin 11 mer, AMGGLLFLLGF (SEQ ID NO: 34); Tetraspanin 9mer, AMGGLLFLL (SEQ ID NO: 35); GRM2 (Glutamate receptor) 11 mer, AMGSLLALLAL (SEQ ID NO: 36); GRM2 9mer, AMGSLLALL (SEQ ID NO: 37); ZNF780B (Zinc finger protein 780B isoform X1) 11 mer, KAFGLLTQLAQ (SEQ ID NO: 38); ZNF780B 9mer, KAFGLLTQL (SEQ ID NO: 39) in (B) and Ctrl PP, GILGFVFTL (SEQ ID NO: 23); LMP-2A 9mer, CLGGLLTMV (SEQ ID NO: 19); EntS 9mer, LLGGLLTMV (SEQ ID NO: 21); mnhA1 9mer, KLGGLLTIM (SEQ ID NO: 40); NhaB 9mer, ALVGGLLMV (SEQ ID NO: 41); PA14 60530 9mer, ALGGVMTMV (SEQ ID NO: 42); TMEM161A 9mer, ALGGLLTPL (SEQ ID NO: 43) in FIG. 15C. Jurkat cells expressing the control TCR (TCR-fluM1, PDB 5euo) are stimulated with the cognate 9mer “GILGFVFTL” (SEQ ID NO: 23) for comparison. ***, p<0.001 by t test.

FIG. 16 summarizes the results of analyses performed to show that TMEM161A is a non-mutated tumor antigen. Left panel: percentages of all types of genetic alterations within the TMEM161A locus defined with whole-genome sequencing from the Cancer Genome Atlas (TCGA) project (pan-lung cancer cases, n=1053) are shown. Right panel: number of cases with indicated genomic alterations.

FIGS. 17A-17C schematically summarize the experimental workflow for the characterization of TMEM161A-specific CD8+ T cells. In these experiments, TMEM9mer/A02 tetramer+ sorted T cells from tumor and uninvolved lung carry the “S % DGMNTE” (SEQ ID NO: 48) motif, as predicted by GLIPH2 were used. FIG. 17A: Sorting HLA-A*02/TMEM9mer+CD8+ T cells from the resected tumor and uninvolved lung of NSCLC patient A6 through FACS. Single cell suspensions were prepared and stained with anti-CD4, CD8, CD3, and CD45 antibodies, live/dead marker AquaZombie, PE-conjugated tetramer HLA-A*02/viral peptides (CMV-pp65495-503 and EBV-BMLF1280-288), and APC-conjugated tetramer HLA-A*02/TMEM9mer. Specific T cells were sorted onto 96-well plates based on the following criteria: non-doublets, live cells, CD3+CD45+, HLA-A*02/viral peptides-, and HLA-A*02/TMEM9mer+. FIG. 17B: Percentages of distinct CDR3β sequences of tetramer-sorted CD8+ T cells as in FIG. 17A from uninvolved lung and tumor are shown. Numbers in bars represent the counts of sorted cells.

FIGS. 18A-18C schematically summarize the results of experiments performed to demonstrate that the frequency of T cells with the “S % DGMNTE” (SEQ ID NO: 48) CDR3β motif in tumors correlate with tumor attributes (FIG. 18A) Numbers of A02+ squamous cell lung carcinoma (LUSC) or lung adenocarcinoma (LUAD) patients from the MD Anderson cohort with or without detected T cells carrying the “S % DGMNTE” (SEQ ID NO: 48) CDR3β motif p value=5.3×10-3 by Fisher's Exact test. (FIG. 18B) Total percentage (log 10) of T cells carrying the “S % DGMNTE” (SEQ ID NO: 48) CDR3β motif for A02+ patients from the MD Anderson cohorts stratified as having high (>500) or moderate to low (<500) mutation counts (n=34).

FIG. 19A depicts a break down of the clinical data of the MD Anderson NSCLC patient cohort stratified by detection of CDR3β sequences carrying the “S % DGMNTE” sequence motif (SEQ ID NO: 48). FIG. 19B shows that there are no differences in overall survival (left) or recurrence-free survival (right) among patients with tumor-infiltrating T cell CDR3β containing the “S % DGMNTE” sequence motif (SEQ ID NO: 48) (n=32) vs patients without tumor-infiltrating T cell CDR3β containing “S % DGMNTE” sequence motif (SEQ ID NO: 48) (n=49).

FIGS. 20A-20B schematically summarize the results of experiments showing that some TMEM161A-specific T cells found in healthy donor peripheral blood have effector phenotype. FIG. 20A: Seurat analysis of the scRNA-Seq results from the sorted CD45+CD8+CD3+ T cells from PBMC with indicated HLA-A*02 tetramers (left) as in FIG. 5A identified 3 major cell states by the UMAP dimensionality reduction method (right). Viral, fluM1 peptide GILGFVFTL. FIG. 20B: Stacked violin plot showing the differential genes that are expressed by the identified cell states.

FIG. 21 schematically summarize the results of analyses performed to further characterize the TMEM161-specific CD8+ cells. This figure shows a dimension reduction by UMAP of a previously published NSCLC scRNA-Seq results (Guo X. et al. 2018. Nat. Med. 24, 978). 12,346 sorted T cells from the Guo et al. publication were combined with the 2,950 sorted T cells from the current study (Stanford cohort) for the joint Seurat analysis. Cells from the Zhang group study are colored according to the identified cell states as reported (left) in comparison with cells from the Stanford cohort colored with the 14 cell states identified in the current study (right). *, cell states identified in the Zhang group report (left) that mostly resembled at least one of the cell states identified in the current study (*, right).

FIGS. 22A-22B summarize the results from experiments performed to demonstrate that TMEM161A protein is expressed on multiple human cancers. A tissue microarray consisting of over 100 human cancer tissues and normal tissues from paraffin-embedded sections were stained anti-TMEM161A antibody. Tissues were manually scored based on percent positivity and intensity for determination of H scores. High levels of TMEM161A expressed were observed in colon cancer, breast cancer, kidney cancer, lung cancer, ovarian cancer, pancreatic cancer, prostate cancer, sarcoma, neuroendocrine cancer, and testicular cancer. Representative examples of TMEM161A expression in cancer tissue are shown, as well as quantification of TMEM161A expression by H-score.

FIG. 23A schematically summarize the results of experiments demonstrating that tumor-derived clone TCR15 is cross-reactive to a shared tumor antigen (CLDN2) and a viral antigen from EBV. Representative FACS plots shown the stimulation of the Jukat-TCR cells with 9 mers from the EBV BMLF1 locus “GLCTLVAML” (SEQ ID NO: 44), uniprot NP 001164563.1 (CLDN2 locus, LLGTLVAML; SEQ ID NO: 45), XP 016864815.1 (SERINC5 locus, YLCTLVAPL; SEQ ID NO: 46), and NP 001005209.1 (TMEM198 locus, HPVGEASIL; SEQ ID NO: 47). Right panel: results of Jurkat-TCR15 cell stimulation in triplicate. Control peptide: flu M1 “GILGFVFTL” (SEQ ID NO: 23). *** p<0.001; **, p<0.01 by student t test.

### DETAILED DESCRIPTION OF THE DISCLOSURE

The present disclosure generally relates to, inter alia, compositions and methods for the diagnosis, prevention, and/or treatment of health conditions. More particularly, provided herein are novel polypeptide constructs having binding affinity for a specific cognate antigen. The disclosure also provides compositions and methods useful for producing such polypeptide constructs as well as methods for the diagnosis, prevention, and/or treatment of conditions associated with cells expressing the cognate antigen recognized by the polypeptide constructs. In particular, also provided are recombinant cells such as lymphocyte T cells that have been engineered to express a polypeptide construct as disclosed herein and are directed against a cell of interest such as a cancer cell.

As will be discussed more thoroughly below, the present disclosure describes an approach that combines bioinformatics and antigen screening to identify novel shared tumor antigens in lung cancer. In some embodiments, the disclosed approach implements an improved version of the algorithm GLIPH (Grouping of Lymphocyte Interactions with Paratope Hotspots), GLIPH2 (Glanville, J., et al., Nature, 2017. 547(7661): p. 94-98; and Huang, H., et al., Nat Biotechnol, 2020. October; 38(10): 1194-1202), to infer the T cell specificities for shared antigens at a global level. Using TCR repertoires from 178 HLA-typed lung cancer patients, GLIPH2 identified over 400 specificity groups inferred to recognize shared tumor antigens in defined HLA contexts. Subsequent analyses were then performed on those with inferred HLA-A*02 restrictions, which informed the prioritization of a particular specificity group carrying the motif “S % DGMNTE” (SEQ ID NO: 48) for antigen identification. In addition, the cognate antigens recognized by the candidate specificity group, including a peptide from the human TMEM161A protein as well as from Epstein-Barr virus (EBV) and E. coli, was also identified. Furthermore, it was observed that TMEM161A to be widely overexpressed in human lung cancer cells. Taken together, the approach described herein has applied a robust method for inferring T cell specificities in lung cancer to identify a novel class of cross-reactive T cells to specific tumor antigen TMEM161A and pathogens.

As discussed above, the wide use of immune checkpoint blockade and T cell-based immunotherapies to treat patients with solid tumors requires a deeper understanding of the T cell specificities in cancer. However, the specificities of the vast majority of tumor-infiltrating T cells remain unknown across all solid tumors despite the availability of advanced technologies for profiling T cell states and repertoires using single-cell sequencing techniques. In recent years, the handful specificities of tumor-infiltrating T cells that have been previously described include T cells recognizing mutated antigens, non-mutated (shared) antigens, and viral antigens. In the era of immune checkpoint blockade, there has been a recent focus on mutated antigens (e.g., neoantigens). As neoantigens represent a type of “altered self” antigen, T cells recognizing this class of antigens have been shown to exhibit an activated phenotype and respond vigorously in tumors. Non-mutated tumor antigens include differentiation antigens (e.g. melanoma-associated antigens) that are expressed in normal tissue counterparts, or self-antigens where expression is restricted to immune-privileged sites, germline tissue, or embryos. There have been numerous examples targeting these types of tumor antigen with adoptive T cell therapies. In addition, T cells with specificities for viruses (such as HPV, EBV, and Merkel cell polyomavirus) have also been a focus of investigation for virus-associated cancers.

In contrast, the role of other types of T cell specificities in solid tumors remains elusive. For example, numerous reports have described the existence of virus-specific T cells in tumors, such as T cells specific for influenza virus (Flu) or cytomegalovirus (CMV) in lung cancer. Without direct evidence of such viruses playing a role in the oncogenesis of lung cancer or other solid tumors, they have largely been presumed to be irrelevant to the tumor immune response and assumed to be “bystander cells”. As described in greater detail below, experimental data described herein have identified a class of specific CD8+ T cells and their cross-reactive antigens from cancer cells and pathogens. This finding is consistent with the hypothesis that maintaining a broad T cell repertoire to defend against viruses and other pathogens may rely on cross-reactivity. T cells specific to self-antigens have been detected in the peripheral blood of healthy individuals, pruned but not clonally deleted in the thymus, potentially to avoid immunologic “blind spots” to viruses and other pathogens. Because cancer cells histologically resemble their tissue of origin and can express self-antigens, we considered the possibility that some tumor-infiltrating T cells are indeed specific to ubiquitously expressed, non-mutated self-antigens. Comprehensively profiling and deep characterization of T cell specificities within the tumor microenvironment provides a fundamental understanding of the T cell response beyond phenotypic characterization and sheds important insight on how the immune system recognizes tumors, normal tissues, and pathogens.

The vast majority of tumor-infiltrating T cells remain unknown is largely due to the absence of tools for analyzing diverse TCR repertoires in the context of highly polymorphic human leukocyte antigens (HLA) alleles. For example, while next-generation sequencing technologies have made the sequencing of large numbers of TCR relatively straightforward and inexpensive, a major problem revolves around how these very large repertoires can be analyzed.

This is because there can be hundreds or thousands of possible TCR sequences for the same peptide-MHC specificity. GLIPH algorithms (Glanville et al., 2018), and more recently an improved version (GLIPH2; Huang et al, Nat Biotechnol, 2020. October; 38(10): 1194-1202) have been previously developed to systemically profile antigen specificities of T cells and to allow inferences of T cell specificity solely based on the CDR3β sequences. These algorithms analyze large numbers of sequences quickly and parse them into TCR specificity groups (a.k.a. specificity groups) that can predict the likely MHC allele restriction. As described in greater detail below, GLIPH2 was used to analyze 778,938 distinct TCRβ CDR3 sequences (referred to as CDR3β sequences) from 178 HLA-typed, non-small cell lung cancer (NSCLC) patients with surgically resectable disease. A total of 4,300 high-confidence specificity groups were initially derived. Of those, 449 were found enriched in tumor compared to uninvolved lung tissue. It was also found that up to 35% of all tumor-infiltrating T cell repertoires within a patient were inferred to have shared antigen specificities. Subsequently, select specificity groups were validated by identifying novel clonotypes predicted to recognize known viral antigens in given HLA contexts and experimentally confirmed these predictions. Next, a specificity group was prioritized that was preferentially enriched in tumor and inferred to recognize antigen in the context of HLA-A*02, and then identified the cognate antigens with yeast display libraries. It was also found that the TCR belonging to this tumor-enriched specificity group recognized the novel tumor antigen TMEM161A. Remarkably, it was also found that this TMEM161A-specific TCR was cross-reactive to similar peptides from pathogens EBV and E. coli. This finding suggests that some pathogen-specific T cells residing in tumors can cross-react to antigens overexpressed in cancer. Phenotypically, these cross-reactive CD8+ T cells adopted an effector cell state, expressing some genes found on activated NK cells and did not express exhaustion markers PD-1 or CD39. In summary, we offer direct evidence that the T cells infiltrating tumors may cross-react to recognize tumor antigens and pathogen-derived antigens.

As described in greater detail below, the experimental data disclosed herein establishes a novel approach for discovering shared tumor antigens and the T cells that recognize them. In particular, as a non-limiting exemplification of the new approach, experiments have been performed to identify a group of cross-reactive TCRs to a tumor antigen associated with lung cancer (TMEM161A), the viral EBV antigen LMP2, and E. coli EntS peptide, which in turn offers an important perspective on how pathogens shape the adaptive immune system response to cancer.

A non-limiting workflow for the approach for discovering novel shared tumor antigens in a target cancer, e.g., lung cancer, generally begins with comprehensive profiling of the T cell specificity landscape in human lung cancer. The bioinformatics tool GLIPH2 was used to profile 778,938 CDR3β sequences from 178 patients and establish 449 tumor-enriched specificity groups. One such TCR with inferred specificity for a shared tumor antigen in the context of HLA-A*02 was identified, and a HLA-A*02 yeast display libraries was screened to identify its cognate antigens. The platform for T cell antigen identification as disclosed herein brings together two technologies. First, the GLIPH2 algorithm performs unbiased inferences of global T cell specificities with accurate predictions of HLA restriction. More information regarding the GLIPH2 algorithm can be found in Huang et al., Nat Biotechnol, 2020. October; 38(10): 1194-1202, the content of which is expressed incorporated by reference. The inferences of shared specificity and HLA context are used to prioritize disease-relevant TCR candidates for downstream antigen discovery. Second, the rich diversity of yeast display libraries greatly facilitates antigen identification and allows for discovery of cross-reactive antigens. Unlike other MHC/peptide libraries built in mammalian cells, the yeast display libraries used the experiments described below incorporate more than 10⁸ randomly permutated peptide sequences. Previously, the uncertainty of HLA restriction limited the success of antigen identification using the yeast display libraries. The studies described herein overcome this limitation by using GLIPH2 algorithm to infer the correct HLA context of the candidate TCR prior to screening the yeast library for its antigens.

As discussed above, uncovering the specificities of tumor-infiltrating T cells is important for understanding how T cell-intrinsic factors shape tumor-immune system interactions and impact therapies aimed at harnessing T cell responses against cancer. Complementing the current understanding of T cell exhaustion as a mechanism of tumor immune evasion, the studies described herein demonstrate that T cell specificities for self-antigens also play a role. Without being bound to any particular theory, it is believed that T cell specificity for self-antigens partly explain why previous studies observed low reactivities of tumor-infiltrating T cells to autologous tumor. As described in greater detail below, a TCR specificity group has been identifying for its ability to recognize the novel shared tumor antigen TMEM161A that is over-expressed in NSCLC. Remarkably, it was observed that despite being highly represented in tumors, TMEM161A-specific T cells are relatively weak responders to the self-antigen TMEM161A compared to the cross-reactive antigens LMP2 and EntS. As shown in the Examples section, TMEM161A-specific T cells were consistently identified in tumors that abundantly express the TMEM161A protein, indicating that antigen-expressing tumor cells are not eliminated in the tested patient. Together, these results indicate that T cells with specificity for some non-mutated tumor antigens are intrinsically weak responders.

In the studies described herein, CD8+ T cells capable of recognizing the tumor antigen TMEM161A and pathogen-derived antigens from EBV LMP2 and E. coli EntS were also identified. This finding is consistent with the observation that self-specific T cells exist in the periphery (and are not deleted in the thymus) to maintain a diverse TCR repertoire capable of responding to foreign pathogens through cross-reactivity. However, it remains largely unknown whether these T cells play an important role in anti-tumor immune responses. The scRNA-seq data described below indicate that HLA-A*02/TMEM9mer-specific T cells in tumors exhibit an effector phenotype differentially expressing EOMES and KLRG1 rather than an exhausted state. The lack of CD39 expression is consistent with the phenotype of the previously reported “bystander” pathogen-specific T cells found in tumors. The experimental data described herein indicates that T cell specificity for tumor antigen and pathogen-derived antigens within tumors are not mutually exclusive. In addition to categorizing T cell specificities in tumors as being tumor-specific or pathogen-specific, the studies described herein suggest a third category of cross-reactive T cells that recognize tumor antigens and pathogen-derived antigens. These cross-reactive T cells have not been previously appreciated.

In addition, the concept that immunologic exposure to environmental pathogens may influence the immune response to tumors has been previously theorized, although its mechanism is poorly understood. As early as the late 19th century, William Coley pioneered a mixed bacterial vaccine termed Coley's toxin for the treatment of cancer patients with some successes. In the modem era, Bacillus Calmette-Guerin (BCG) is routinely used as an immunotherapy for early-stage bladder cancer. Recently the gut microbiome has been shown to be a key determinant of immunotherapy responses in cancer. In pancreatic cancer, a unique microbiome has been observed in patients with longest survival after surgery. While the mechanism of action of these various examples could involve cell types of the innate immune system, cross-reactive T cells recognizing both tumor and pathogens might be playing an essential role. Furthermore, as the lungs are exposed to respiratory pathogens, it is contemplated that the cross talk between these antigens and tumor antigens is particularly important for understanding the adaptive immune responses to lung cancer.

Experimental results described herein have demonstrated that the categorization of T cell specificities in tumors as tumor-specific or as pathogen-specific bystanders does not fully capture all possibilities for T cell antigen recognition. As described in greater detail below, T cells in tumors can also be cross-reactive to both tumor antigens and pathogen-derived antigens and therefore offers a more nuanced understanding of T cell specificity in tumors. The disclosed approach for finding this particular class of TCRs also demonstrates a novel methodology for discovering additional tumor antigens. This is because a deeper understanding of how cross-reactive T cells recognize tumor antigens and pathogen-derived antigens can inform advancements in cellular therapies, checkpoint therapies, and vaccination strategies against cancer. The experimental data disclosed herein indicates that an individual's encounters with environmental pathogens may shape the adaptive immune response against cancer, a concept that can be harnessed for improving immunotherapies for patients.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

The term “effective”, “therapeutically effective”, or “pharmaceutically effective” amount or number of a subject construct, nucleic acid, cell, or composition of the disclosure generally refer to an amount or number sufficient for a construct, nucleic acid, cell, or composition to accomplish a stated purpose relative to the absence of the composition (e.g., achieve the effect for which it is administered, prevent or treat a disease, inhibit a microbial infection, or reduce one or more symptoms of a health condition). An example of an effective amount or number is an amount or number sufficient to contribute to the treatment, prevention, or reduction of a symptom or symptoms of a disease, which could also be referred to as a therapeutically effective amount. A “reduction” of a symptom(s) generally means decreasing of the severity or frequency of the symptom(s), or elimination of the symptom(s). The exact amount or number of a construct, nucleic acid, cell, or composition will depend on the purpose of the treatment, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription. It should be understood that, operably linked elements may be contiguous or non-contiguous.

In the context of polypeptide constructs, “operably linked” refers to a physical linkage (e.g., directly or indirectly linked) between amino acid sequences (e.g., different segments, portions, or domains) to provide for a described activity of the constructs. In the present disclosure, region, or domains of the constructs of the disclosure may be operably linked to retain proper folding, processing, targeting, expression, binding, and other functional properties of the constructs in the cell. Unless stated otherwise, the segments, portions, and domains of the constructs of the disclosure are operably linked to each other. Operably linked segments, portions, and domains of the constructs disclosed herein may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a query sequence. This definition includes sequence comparison performed by a BLAST algorithm wherein the parameters of the algorithm are selected to give the largest match between the respective sequences over the entire length of the respective reference sequences. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### T Cell Receptors

A TCR is a heterodimeric cell surface protein of the immunoglobulin super-family, which is associated with invariant proteins of the CD3 complex involved in mediating signal transduction. TCRs and antibodies are molecules that have evolved to recognize different classes of antigens (ligands). TCRs are antigen-specific molecules that are responsible for recognizing antigenic peptides presented in the context of a product of the major histocompatibility complex (MHC) on the surface of antigen presenting cells (APCs) or any nucleated cell (e.g., all human cells in the body, except red blood cells). In contrast, antibodies generally recognize soluble or cell-surface antigens, and do not require presentation of the antigen by an MHC. This system endows T cells, via their TCRs, with the potential ability to recognize the entire array of intracellular antigens expressed by a cell (including viral and bacterial proteins) that are processed intracellularly into short peptides, bound to an intracellular MHC molecule, and delivered to the surface as a peptide-MHC complex (pepMHC). This system allows virtually any foreign protein (e.g., mutated cancer antigen or virus protein) or aberrantly expressed protein to serve a target for T cells.

Generally, TCRs exist in αβ and γδ forms, which are structurally similar but have quite distinct anatomical locations and probably functions. The extracellular portion of native heterodimeric αβ TCR generally consists of two polypeptides, an a chain and a p chain, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies, embedded in a framework sequence, one being the hyper-variable region named CDR3. There are several types of alpha chain variable (Vu) regions and several types of beta chain variable (VO) regions distinguished by their framework, CDR1 and CDR2 sequences, and by a partly defined CDR3 sequence. The use of TCR gene therapy overcomes a number of current hurdles. For example, it allows equipping patients' own T cells with desired specificities and generation of sufficient numbers of T cells in a short period of time, avoiding their exhaustion. In addition, the TCR can be transduced into central memory T cells or T cells with stem cell characteristics, which may ensure better persistence and function upon transfer. Furthermore, TCR-engineered T cells can be infused into cancer patients rendered lymphopenic by chemotherapy or irradiation, allowing efficient engraftment but inhibiting immune suppression.

### Compositions of the Disclosure

As described in greater detail below, one aspect of the present disclosure relates to novel polypeptide constructs having binding affinity for a specific cognate antigen. Also provided are recombinant nucleic acids encoding such polypeptide constructs, as well as recombinant cells that have been engineered to express a polypeptide construct as disclosed herein and are directed against a cell of interest such as a cancer cell.

### A. Constructs of the Disclosure

In one aspect, provided herein are various constructs including at least one complementary determining region (CDR) having at least 70% sequence identity to a sequence selected from the group consisting of SEQ ID NOs: 1-14, 26-33, and 48-49.

Non-limiting exemplary embodiments of the disclosed constructs can include one or more of the following features. In some embodiments, the constructs include at least one, at least two, or at least three CDR having at least 70% sequence identity to a sequence selected from the group consisting of SEQ ID NOs: 1-14 and 48. In some embodiments, the constructs include at least one, at least two, or at least three CDR having at least 70% sequence identity to the sequence of SEQ ID NO: 6. In some embodiments, the constructs include at least one, at least two, or at least three CDR having at least 70% sequence identity to the sequence of SEQ ID NO: 48. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to a sequence selected from the group consisting of SEQ ID NOs: 1-14 and 48. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 6. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 48.

In some embodiments, the constructs include at least one, at least two, or at least three CDR having at least 70% sequence identity to a sequence selected from the group consisting of SEQ ID NOs: 26-33 and 49. In some embodiments, the constructs include at least one, at least two, or at least three CDR having at least 70% sequence identity to the sequence of SEQ ID NO: 30. In some embodiments, the constructs include at least one, at least two, or at least three CDR having at least 70% sequence identity to the sequence of SEQ ID NO: 49. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to a sequence selected from the group consisting of SEQ ID NOs: 26-33. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 30. In some embodiments, the constructs include at least one CDR having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 49.

The CDR sequence of the constructs disclosed herein may be modified, e.g., mutated. Non-limiting examples of modifications of the CDR sequence include a substitution, a deletion, an addition, or an insertion of no more than five, no more than four, no more than three, no more than two, or no more than one amino acid residue. In some embodiments, the at least one CDR of the constructs disclosed herein includes a sequence having 100% identity to a sequence selected from the group consisting of SEQ ID NOs: 1-14 and 48, wherein at least 1, at least 2, at least 3, at least 4, at least 5 amino acid residues in the sequence is substituted by a different amino acid residue. In some embodiments, the at least one CDR of the constructs disclosed herein includes a sequence having 100% identity to the sequence of SEQ ID NO: 6, wherein at least 1, at least 2, at least 3, at least 4, at least 5 amino acid residues in the sequence is substituted by a different amino acid residue. In some embodiments, the at least one CDR of the constructs disclosed herein includes a sequence having 100% identity to the sequence of SEQ ID NO: 48, wherein at least 1, at least 2, at least 3, at least 4, at least 5 amino acid residues in the sequence is substituted by a different amino acid residue.

In some embodiments, the at least one CDR of the constructs disclosed herein includes a sequence having 100% identity to a sequence selected from the group consisting of SEQ ID NOs: 26-33 and 49, wherein at least 1, at least 2, at least 3, at least 4, at least 5 amino acid residues in the sequence is substituted by a different amino acid residue. In some embodiments, the at least one CDR of the constructs disclosed herein includes a sequence having 100% identity to the sequence of SEQ ID NO: 30, wherein at least 1, at least 2, at least 3, at least 4, at least 5 amino acid residues in the sequence is substituted by a different amino acid residue. In some embodiments, the at least one CDR of the constructs disclosed herein includes a sequence having 100% identity to the sequence of SEQ ID NO: 49, wherein at least 1, at least 2, at least 3, at least 4, at least 5 amino acid residues in the sequence is substituted by a different amino acid residue.

In some embodiments, the at least one CDR includes a sequence having 100% identity to a sequence selected from the group consisting of SEQ ID NOs: 1-14 and 48, wherein one, two, three, four, or five of the amino acid residues in the sequence is substituted by a different amino acid residue. In some embodiments, the at least one CDR includes a sequence having 100% identity to the sequence of SEQ ID NO: 6, wherein one, two, three, four, or five of the amino acid residues in the sequence is substituted by a different amino acid residue. In some embodiments, the at least one CDR includes a sequence having 100% identity to the sequence of SEQ ID NO: 48, wherein one, two, three, four, or five of the amino acid residues in the sequence is substituted by a different amino acid residue. In some embodiments, the at least one CDR includes a sequence having 100% identity to a sequence selected from the group consisting of SEQ ID NOs: 26-33 and 49, wherein one, two, three, four, or five of the amino acid residues in the sequence is substituted by a different amino acid residue. In some embodiments, the at least one CDR includes a sequence having 100% identity to the sequence of SEQ ID NO: 30, wherein one, two, three, four, or five of the amino acid residues in the sequence is substituted by a different amino acid residue. In some embodiments, the at least one CDR includes a sequence having 100% identity to the sequence of SEQ ID NO: 49, wherein one, two, three, four, or five of the amino acid residues in the sequence is substituted by a different amino acid residue.

In some embodiments, the construct as disclosed herein is capable of binding to an epitope including a sequence having at least 70% sequence identity to a sequence selected from the group consisting of SEQ ID NOs: 15-22 and 44-45. One of ordinary skill in the art will understand that binding affinity can generally be used as a measure of the strength of a non-covalent interaction between two molecules, e.g., an antibody or functional fragment thereof and an antigen. In some cases, binding affinity can be used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant kₐ (or k_{off}), respectively. K_{D} is related to kₐ and kₐ through the equation K_{D}=k_{d}/kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of engineered antibodies of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA. In some embodiments, the binding affinity of a construct as disclosure herein for a target antigen can be assessed by the Scatchard method described by Frankel et al., Mol. Immunol, 16: 101-106, 1979.

In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to a sequence selected from the group consisting of SEQ ID NOs: 15-22 and 44-45. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 17. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 19. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 21. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 22. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 44. In some embodiments of the disclosure, the construct as disclosed herein is capable of binding to an epitope including a sequence having at least 70%, for example at least 75%, at least 80%, at least 85%, at least 90%, at least 95%, at least 96%, at least 97%, at least 98%, at least 99%, or at least 100% sequence identity to the sequence of SEQ ID NO: 45. In some embodiments, the epitope includes a sequence having 100% identity to a sequence selected from the group consisting of SEQ ID NOs: 15-22 and 44-45, and further having no more than five, no more than four, no more than three, no more than two, or no more than one amino acid residue substituted by a different amino acid residue. In some embodiments, the epitope includes a sequence having 100% identity to SEQ ID NO: 17, and further having no more than five, no more than four, no more than three, no more than two, or no more than one amino acid residue substituted by a different amino acid residue. In some embodiments, the epitope includes a sequence having 100% identity to SEQ ID NO: 19, and further having no more than five, no more than four, no more than three, no more than two, or no more than one amino acid residue substituted by a different amino acid residue. In some embodiments, the epitope includes a sequence having 100% identity to SEQ ID NO: 21, and further having no more than five, no more than four, no more than three, no more than two, or no more than one amino acid residue substituted by a different amino acid residue. In some embodiments, the epitope includes a sequence having 100% identity to SEQ ID NO: 22, and further having no more than five, no more than four, no more than three, no more than two, or no more than one amino acid residue substituted by a different amino acid residue. In some embodiments, the epitope includes a sequence having 100% identity to SEQ ID NO: 44, and further having no more than five, no more than four, no more than three, no more than two, or no more than one amino acid residue substituted by a different amino acid residue.

In some embodiments, the epitope includes a sequence having 100% identity to a sequence selected from the group consisting of SEQ ID NOs: 15-22 and 44-45, wherein at least 1, at least 2, at least 3, at least 4, at least 5 amino acid residues in the sequence is substituted by a different amino acid residue. In some embodiments, the epitope includes a sequence having 100% identity to SEQ ID NO: 17, wherein at least 1, at least 2, at least 3, at least 4, at least 5 amino acid residues in the sequence is substituted by a different amino acid residue. In some embodiments, the epitope includes a sequence having 100% identity to SEQ ID NO: 19, wherein at least 1, at least 2, at least 3, at least 4, at least 5 amino acid residues in the sequence is substituted by a different amino acid residue. In some embodiments, the epitope includes a sequence having 100% identity to SEQ ID NO: 21, wherein at least 1, at least 2, at least 3, at least 4, at least 5 amino acid residues in the sequence is substituted by a different amino acid residue. In some embodiments, the epitope includes a sequence having 100% identity to SEQ ID NO: 22, wherein at least 1, at least 2, at least 3, at least 4, at least 5 amino acid residues in the sequence is substituted by a different amino acid residue. In some embodiments, the epitope includes a sequence having 100% identity to SEQ ID NO: 44, wherein at least 1, at least 2, at least 3, at least 4, at least 5 amino acid residues in the sequence is substituted by a different amino acid residue. In some embodiments, the epitope includes a sequence having 100% identity to SEQ ID NO: 45, wherein at least 1, at least 2, at least 3, at least 4, at least 5 amino acid residues in the sequence is substituted by a different amino acid residue. In some embodiments, the epitope includes a sequence having 100% identity to a sequence selected from the group consisting of SEQ ID NOs: 15-22 and 44-45, wherein one, two, three, four, or five of the amino acid residues in the sequence is substituted by a different amino acid residue. In some embodiments, the epitope includes a sequence having 100% identity to SEQ ID NO: 17, wherein one, two, three, four, or five of the amino acid residues in the sequence is substituted by a different amino acid residue. In some embodiments, the epitope includes a sequence having 100% identity to SEQ ID NO: 19, wherein one, two, three, four, or five of the amino acid residues in the sequence is substituted by a different amino acid residue. In some embodiments, the epitope includes a sequence having 100% identity to SEQ ID NO: 21, wherein one, two, three, four, or five of the amino acid residues in the sequence is substituted by a different amino acid residue. In some embodiments, the epitope includes a sequence having 100% identity to SEQ ID NO: 22, wherein one, two, three, four, or five of the amino acid residues in the sequence is substituted by a different amino acid residue. In some embodiments, the epitope includes a sequence having 100% identity to SEQ ID NO: 44, wherein one, two, three, four, or five of the amino acid residues in the sequence is substituted by a different amino acid residue. In some embodiments, the epitope includes a sequence having 100% identity to SEQ ID NO: 45, wherein one, two, three, four, or five of the amino acid residues in the sequence is substituted by a different amino acid residue.

In some embodiments, the construct of the disclosure can be (a) a TCR; (b) an antibody; or (c) a functional derivative or fragment of (a) or (b). One skilled in the art upon will readily understand that the term “functional fragment thereof” or “functional derivative thereof” refers to a molecule having quantitative and/or qualitative biological activity in common with the wild-type molecule from which the fragment or derivative was derived. For example, a functional fragment or a functional derivative of an antibody is one which retains essentially the same ability to bind to the same epitope as the antibody from which the functional fragment or functional derivative was derived. For instance, an antibody capable of binding to an epitope may be truncated at the N-terminus and/or C-terminus, and the retention of its epitope binding activity assessed using assays known to those of skill in the art.

In some embodiments, the construct is a TCR construct including a TCR alpha chain and a TCR beta chain covalently linked to each other. The present disclosure provides both single-chain TCR constructs and multiple-chain TCR constructs. In some embodiments, the TCR constructs of the disclosure may be provided as single chain α or β, or γ and δ, molecules, or alternatively as double chain constructs composed of both the α and β chain, or γ and δ chain.

In some embodiments, the TCR construct of the disclosure may be provided as a single chain TCR (scTCR). A scTCR can include a polypeptide of a variable region of a first TCR chain (e.g., an alpha chain) and a polypeptide of an entire (full-length) second TCR chain (e.g., a beta chain), or vice versa. In some embodiments, the polypeptides are directly linked to one another. In some embodiments, the scTCR can optionally include one or more linkers which join the two or more polypeptides together. In some embodiments, the linker can be a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In some embodiments, the linker can be a peptide linker, which joins together two single chains, as described herein. In some embodiments, the length and amino acid composition of the peptide linker sequence can be optimized to vary the orientation and/or proximity of the polypeptides relative to one another to achieve a desired activity of the constructs (e.g., TCR constructs) as disclosed herein.

The construct according to the present disclosure can also be provided in the form of a multimeric complex, including at least two scTCR molecules, wherein the scTCR molecules are each fused to at least one biotin moiety, or other interconnecting molecule/linker, and wherein the scTCRs are interconnected by biotin-streptavidin interaction to allow the formation of said multimeric complex. Similar approaches known in the art for the generation of multimeric TCR are also contemplated and included in this disclosure. Accordingly, also provided are multimeric complexes of a higher order, comprising more than two scTCR of the disclosure.

Suitable methods of making fusion polypeptides are known in the art, and include, for example, recombinant methods. In some embodiments, the constructs, TCRs (and functional fragments and functional derivatives thereof), and polypeptides of the disclosure may be expressed as a single protein including a linker peptide linking the a chain and the β chain, and/or linking the γ chain and the δ chain. In this regard, the constructs, TCRs (and functional fragments and functional derivatives thereof), and polypeptides of the disclosure include the amino acid sequences of the variable regions of the TCR of the disclosure and can further include a linker peptide. In some embodiments, the linker peptide may advantageously facilitate the expression of a construct or a TCR (including functional fragments and functional derivatives thereof) in a host cell. In principle, the linker peptide may comprise any suitable amino acid sequence. Linker sequences for single chain TCR constructs are well known in the art. In some embodiments, such a single chain construct can further comprise one, or two, constant domain sequences. Upon expression of the construct including the linker peptide by a host cell, the linker peptide may also be cleaved, resulting in separated α and β chains, and separated γ and δ chain.

In some embodiments, the TCR constructs of the disclosure includes at least one TCR α or γ and/or TCR β or δ variable domain. Generally, they include both a TCR α variable domain and a TCR β variable domain, alternatively both a TCR γ variable domain and a TCR δ variable domain. In some embodiments, the TCR constructs include αβ/γδ heterodimers or may be in single chain format. In some embodiments, for use in adoptive therapy, an αβ or γδ heterodimeric TCR may, for example, be transfected as full length chains having both cytoplasmic and transmembrane domains. If desired, an introduced disulfide bond between residues of the respective constant domains can be present.

In some embodiments, the TCR constructs of the disclosure are provided as single chain α or β, or γ and δ, molecules, or alternatively as double chain constructs composed of both the α and β chain, or γ and δ chain. Accordingly, in some embodiments, the TCR construct is a single-chain TCR construct including in its beta chain a CDR3β having at least 70%, for example, at least 75%, at least 80%, at least 85%, at least 90%, at least 95%, at least 96%, at least 97%, at least 98%, at least 99%, or at least 100% sequence identity to a sequence selected from SEQ ID NOs: 1-14, 26-33, and 48-49. In additional or alternative embodiments, the TCR construct may further include a CDR1 and/or a CDR2 domain sequence. In some embodiments, the TCR constructs of the disclosure include at least one, preferably all three CDR sequences CDR1, CDR2 and CDR3.

In some embodiments, the TCR constructs of the disclosure are provided as double-chain constructs composed of both the α and R chain, or γ and δ chain. Accordingly, in some embodiments, the TCR constructs of the disclosure are provided as double-chain constructs comprising both the α and R chain, wherein its beta chain includes a CDR3β having at least 70%, for example, at least 75%, at least 80%, at least 85%, at least 90%, at least 95%, at least 96%, at least 97%, at least 98%, at least 99%, or at least 100% sequence identity to a sequence selected from SEQ ID NOs: 1-14, 26-33, and 48-49. In some embodiments, the TCR constructs further include in its alpha chain a CDR3α sequence. In some embodiments, the CDR3α sequence has at least 70% sequence identity to SEQ ID NO: 24. In some embodiments, the CDR3α sequence has at least 75%, at least 80%, at least 85%, at least 90%, at least 95%, at least 96%, at least 97%, at least 98%, at least 99%, or at least 100% sequence identity to SEQ ID NO: 24. In some embodiments, the CDR3α includes a sequence having at least 75%, at least 80%, at least 85%, at least 90%, at least 95%, at least 96%, at least 97%, at least 98%, at least 99%, or at least 100% sequence identity to the sequence of SEQ ID NO: 24. In some embodiments, the CDR3α sequence has at least 100% sequence identity to the sequence of SEQ ID NO: 24 and further includes one, two, three, or four amino acid residues of SEQ ID NO: 24 substituted with a different amino acid residue.

As outlined above, in some embodiments, the construct of the disclosure can be provided in the framework of an antibody construct or a functional fragment thereof, which specifically binds to the antigens described herein. The antibody construct can be any type of immunoglobulin that is known in the art. For instance, the antibody construct can be of any iso-type, e.g., IgA, IgD, IgE, IgG, IgM, etc. The antibody construct can be monoclonal or polyclonal. The antibody construct can be a naturally-occurring antibody, e.g., an antibody isolated and/or purified from a mammal, e.g., human cell. Alternatively, the antibody construct can be a genetically-engineered antibody, e.g., a humanized antibody or a chimeric antibody. The antibody construct can be in monomeric or polymeric form. In some embodiments, the construct disclosed herein is an antibody construct selected from the group consisting of an antigen-binding fragment (Fab), a single-chain variable fragment (scFv), a nanobody, a single domain antibody (sdAb), a V_{H} domain, a V_{L} domain, a V_{H}H domain, a diabody, or a functional fragment of any thereof.

### B. Nucleic Acids

In one aspect, provided herein are various nucleic acid molecules including nucleotide sequences encoding the constructs of the disclosure, including expression cassettes, and expression vectors containing these nucleic acid molecules operably linked to heterologous nucleic acid sequences such as, for example, regulator sequences which allow in vivo expression of the constructs in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments disclosed herein, the nucleic acid molecules of the disclosure include a nucleotide sequence encoding a construct including at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-14, 26-33, and 48-49. In some embodiments, the construct is capable of binding to an epitope including a sequence having at least 70%, 75%, 80%, 85%, 90%, 95%, 96%, 97%, 98%, 99%, or 100% sequence identity to a sequence selected from the group consisting of SEQ ID NOs: 15-22 and 44-45. In some embodiments, the construct is single-chain constructs or double-chain constructs. In some embodiments, the construct is selected from the group consisting of: (a) a TCR; (b) an antibody; and (c) a functional derivative or fragment of (a) or (b). In some embodiments, the construct is a TCR construct including a TCR alpha chain and a TCR beta chain covalently linked to each other. In some embodiments, the construct is a TCR construct including in its beta chain a CDR3β having at least 70%, 75%, 80%, 85%, 90%, 95%, 96%, 97%, 98%, 99%, or 100% sequence identity to a sequence selected from SEQ ID NOs: 1-14, 26-33, and 48-49. In some embodiments, the construct further includes in its alpha chain a CDR3α sequence. In some embodiments, the CDR3α has at least 70%, 75%, 80%, 85%, 90%, 95%, 96%, 97%, 98%, 99%, or 100% sequence identity to the sequence of SEQ ID NO: 24. In some embodiments, the CDR3α sequence has at least 100% sequence identity to the sequence of SEQ ID NO: 24 and further includes one, two, three, or four amino acid residues of SEQ ID NO: 24 substituted with a different amino acid residue.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the construct as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a baculoviral vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acid molecules encoding any of the constructs disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan.

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, baculoviral vectors, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoamidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In another aspect, provided herein are cell cultures including at least one engineered cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one engineered cell as disclosed herein are also within the scope of this application. Methods and systems suitable for generating and maintaining cell cultures are known in the art.

### C. Engineered Cells and Cell Cultures

The recombinant nucleic acids of the present disclosure can be introduced into a cell, such as, for example, a human T lymphocyte, to produce an engineered cell containing the nucleic acid molecule. Accordingly, some embodiments of the disclosure relate to methods for making an engineered cell, including (a) providing a host cell capable of protein expression; and transducing the provided host cell with a recombinant nucleic acid of the disclosure to produce an engineered cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce an engineered cell. For example, the nucleic acid molecule can be stably integrated in the engineered cell's genome, or can be episomally replicating, or present in the engineered cell as a mini-circle expression vector for transient expression. Accordingly, in some embodiments, the nucleic acid molecule is maintained and replicated in the recombinant host cell as an episomal unit. In some embodiments, the nucleic acid molecule is present in the engineered cell as a mini-circle expression vector for transient expression. In some embodiments, the nucleic acid molecule is stably integrated into the genome of the engineered cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the engineered cell is a prokaryotic cell or a eukaryotic cell. In some embodiments, the cell is in vivo. In some embodiments, the cell is ex vivo. In some embodiments, the cell is in vitro. In some embodiments, the engineered cell is a eukaryotic cell. In some embodiments, the engineered cell is an animal cell. In some embodiments, the animal cell is a mammalian cell. In some embodiments, the animal cell is a human cell. In some embodiments, the cell is a non-human primate cell. In some embodiments, the engineered cell is an immune system cell, e.g., a B cell, a monocyte, a NK cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+ T cytotoxic lymphocyte cell. Non-limiting examples of CD8+ T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell. Suitable CD4+ T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

As outlined above, some embodiments of the disclosure relate to various methods for making an engineered cell of the disclosure, the methods include: (a) providing a host cell capable of protein expression; and transducing the provided host cell with a recombinant nucleic acid of the disclosure to produce an engineered cell. Non-limiting exemplary embodiments of the disclosed methods for making an engineered cell can further include one or more of the following features. In some embodiments, the cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the engineered cells produced by the methods disclosed herein are also within the scope of the disclosure.

In another aspect, provided herein are cell cultures including at least one engineered cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one engineered cell as disclosed herein are also within the scope of this application. Methods and systems suitable for generating and maintaining cell cultures are known in the art.

### E. Pharmaceutical Compositions

The constructs, nucleic acids, engineered cells, and/or cell cultures of the disclosure can be incorporated into compositions, including pharmaceutical compositions. Such compositions generally include one or more of the constructs, nucleic acids, engineered cells, and/or cell cultures as provided and described herein, and a pharmaceutically acceptable excipient, e.g., carrier. In some embodiments, the pharmaceutical compositions of the disclosure are formulated for the treating, preventing, ameliorating a disease such as cancer, or for reducing or delaying the onset of the disease.

Accordingly, one aspect of the present disclosure relates to pharmaceutical compositions that include a pharmaceutically acceptable carrier and one or more of the following: (a) a construct of the disclosure; (b) a recombinant nucleic acid of the disclosure; and (c) an engineered cell of the disclosure. In some embodiments, the pharmaceutical compositions include (a) a construct of the disclosure and (b) a pharmaceutically acceptable carrier. In some embodiments, the pharmaceutical compositions include (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the pharmaceutical compositions of the disclosure include (a) an engineered cell of the disclosure and (b) a pharmaceutically acceptable carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Methods of the Disclosure

Administration of any one of the therapeutic compositions described herein, e.g., constructs, nucleic acids, engineered cells, and pharmaceutical compositions, can be used to treat subjects in the treatment of relevant diseases, such as cancers, immune diseases, and chronic infections. In some embodiments, the constructs, nucleic acids, engineered cells, and pharmaceutical compositions as described herein can be incorporated into therapeutic agents for use in methods of preventing and/or treating a subject who has, who is suspected of having, or who may be at high risk for developing one or more health conditions, such as proliferative disorders or microbial infections. Exemplary proliferative disorders can include, without limitation, angiogenic diseases, a metastatic diseases, tumorigenic diseases, neoplastic diseases and cancers. In some embodiments, the proliferative disorder is a cancer.

Accordingly, in one aspect, some embodiments of the disclosure relate to methods for the prevention and/or treatment of a condition in a subject in need thereof, wherein the methods include administering to the subject a composition including one or more of: (a) a construct of the disclosure; (b) a recombinant nucleic acid of the disclosure; (c) an engineered cell of the disclosure; and d) a pharmaceutically composition of the disclosure. In some embodiments, the composition includes a therapeutically effective amount or number of: (a) a construct of the disclosure; (b) a recombinant nucleic acid of the disclosure; (c) an engineered cell of the disclosure; and/or a pharmaceutical composition of the disclosure.

In some embodiments, the disclosed pharmaceutical composition is formulated to be compatible with its intended route of administration. The recombinant polypeptides of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant polypeptides of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutically effective amount of a subject recombinant polypeptide of the disclosure (e.g., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU). The compositions can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject recombinant polypeptides of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

### Administration of Engineered Cells to a Subject

In some embodiments, the methods of treatment as disclosed herein involve administering an effective amount or number of the engineered cells to a subject in need of such treatment. This administering step can be accomplished using any method of implantation delivery in the art. For example, the engineered cells can be infused directly in the individual's bloodstream or otherwise administered to the individual.

In some embodiments, the methods disclosed herein include administering, which term is used interchangeably with the terms “introducing,” implanting,” and “transplanting,” engineered cells into a subject, by a method or route that results in at least partial localization of the introduced cells at a desired site such that a desired effect(s) is/are produced. The engineered cells or their differentiated progeny can be administered by any appropriate route that results in delivery to a desired location in the individual where at least a portion of the administered cells or components of the cells remain viable. The period of viability of the cells after administration to a subject can be as short as a few hours, e.g, twenty-four hours, to a few days, to as long as several years, or even the lifetime of the individual, i.e., long-term engraftment.

When provided prophylactically, the engineered cells described herein can be administered to a subject in advance of any symptom of a disease or condition to be treated. Accordingly, in some embodiments the prophylactic administration of an engineered cell population prevents the occurrence of symptoms of the disease or condition.

When provided therapeutically in some embodiments, engineered cells are provided at (or after) the onset of a symptom or indication of a disease or condition, e.g., upon the onset of disease or condition.

For use in the various embodiments described herein, an effective amount or number of engineered cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The engineered cells can be derived from one or more donors or can be obtained from an autologous source. In some embodiments, the engineered cells are expanded in culture prior to administration to a subject in need of a treatment.

In some embodiments, the delivery of an engineered cell composition (e.g., a composition including a plurality of engineered cells according to any of the cells described herein) into an individual by a method or route results in at least partial localization of the cell composition at a desired site. A composition including engineered cells can be administered by any appropriate route that results in effective treatment in the individual, e.g., administration results in delivery to a desired location in the individual where at least a portion of the composition delivered, e.g., at least 1×10³ cells, is delivered to the desired site for a period of time. Modes of administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the engineered cells are administered systemically, e.g., via infusion or injection. For example, a population of engineered cells are administered other than directly into a target site, tissue, or organ, such that it enters the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective number of engineered cells refers to a number of engineered cells that is sufficient to promote a provide a therapeutic benefit in the treatment or management of a disease, e.g., cancer, or to delay or minimize one or more symptoms associated with the disease when administered to a subject, such as one who has, is suspected of having, or is at risk for the disease. In some embodiments, an effective number includes a number sufficient to prevent or delay the development of a symptom of the disease, alter the course of a symptom of the disease (for example but not limited to, slow the progression of a symptom of the disease), or reverse a symptom of the disease. In some embodiments, an effective number includes a number sufficient to inhibit tumor growth or metastasis of a cancer in the individual. In some embodiments, an effective number includes a number sufficient to increase cytokine production, inhibit (e.g., kill) a cancer cell or an infected cell.

In some embodiments of the disclosed methods, the individual is a mammal. In some embodiments, the mammal is a human. In some embodiments, the individual has or is suspected of having a condition associated with a proliferative disorder or disease, such as a cancer. The term cancer generally refers to the presence of cells possessing characteristics typical of cancer-causing cells, such as uncontrolled proliferation, immortality, metastatic potential, rapid growth and proliferation rate, and certain characteristic morphological features. Cancer cells are often observed aggregated into a tumor, but such cells can exist alone within an animal subject, or can be a non-tumorigenic cancer cell, such as a leukemia cell. Thus, the terms “cancer” or can encompass reference to a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

Examples of conditions suitable for being treated by the compositions and methods of the disclosure include those associated with cancers, autoimmune diseases, inflammatory diseases, and infectious diseases. In some embodiments, the proliferative disorder is a cancer or a microbial infection. Non-limiting examples of cancers that can suitably be treated by the compositions and methods of the disclosure include cancers that express an antigen including an epitope having at least 70%, 75%, 80%, 85%, 90%, 95%, 96%, 97%, 98%, 99%, or 100% sequence identity to a sequence selected from the group consisting of SEQ ID NOs: 15-22. In some embodiments, the cancers are characterized by an increased amount and/or activity of an antigen that includes an epitope having at least 70%, 75%, 80%, 85%, 90%, 95%, 96%, 97%, 98%, 99%, or 100% sequence identity to a sequence selected from the group consisting of SEQ ID NOs: 15-22. In some embodiments, the proliferative disorder is a cancer expressing the TMEM161A antigen (e.g., TMEM161A-positive cancer). In some embodiments, the proliferative disorder is a cancer associated with an increased amount and/or activity of the TMEM161A antigen compared to a reference non-cancerous cell, e.g., a cell which is non-cancerous and which is obtained from a matching tissue as the original tissue/cell from which the cancer originates. In some embodiments, the proliferative disorder is a cancer expressing TMEM161A at levels that are at least 10% higher such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times of at least one reference non-cancerous cell.

Examples of cancers that can be suitably diagnosed, prevented, and/or treated by the compositions and methods of the disclosure include colon cancer, breast cancer, kidney cancer, lung cancer, and ovarian cancer. Additional cancers that can be suitably diagnosed, prevented, and/or treated by the compositions and methods of the disclosure include, but are not limited to, pancreatic cancer, prostate cancer, sarcoma, neuroendocrine cancer, and testicular cancer. In some embodiments, the cancer is a lung cancer.

Additional examples of cancers that can suitably be treated by the compositions and methods of the disclosure include cancers that express an antigen including an epitope having at least 70%, 75%, 80%, 85%, 90%, 95%, 96%, 97%, 98%, 99%, or 100% sequence identity to a sequence selected from the group consisting of SEQ ID NOs: 44-45. In some embodiments, the cancers are characterized by an increased amount and/or activity of an antigen that includes an epitope having at least 70%, 75%, 80%, 85%, 90%, 95%, 96%, 97%, 98%, 99%, or 100% sequence identity a sequence selected from the group consisting of SEQ ID NOs: 44-45. In some embodiments, the proliferative disorder is a cancer expressing the CLDN2 antigen (e.g., CLDN2-positive cancer). In some embodiments, the proliferative disorder is a cancer associated with an increased amount and/or activity of the CLDN2 antigen compared to a reference non-cancerous cell, e.g., a cell which is non-cancerous and which is obtained from a matching tissue as the original tissue/cell from which the cancer originates. In some embodiments, the proliferative disorder is a cancer expressing CLDN2 at levels that are at least 10% higher such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times of at least one reference non-cancerous cell.

Additional cancers that can be suitably diagnosed, prevented, and/or treated by the compositions and methods of the disclosure include, but are not limited to, colorectal cancer, cervical cancer, liver cancer, lung cancer, gastric cancer, pancreatic cancer, renal cancer, and stomach cancer.

In some embodiments, the cancer is a lung cancer. In principle, there are no particular limitations to the in regard to the lung cancers that can be suitably diagnosed, prevented, and/or treated by the compositions and methods disclosed herein. Examples of suitable lung cancers include adenocarcinoma, squamous cell carcinoma, small cell carcinoma, non-small cell carcinoma, adenosquamous carcinoma, small cell lung cancer, large cell carcinoma, neuroendocrine cancers of the lung, non-small cell lung cancer (NSCLC). Additional lung cancers that can be suitably diagnosed, prevented, and/or treated by the compositions and methods disclosed herein include, but are not limited to, undifferentiated non-small cell carcinoma, non-small cell carcinoma not otherwise specified, pulmonary squamous cell carcinoma, broncho-alveolar carcinoma, sarcomatoid carcinoma, pleomorphic carcinoma, carcinosarcoma, pulmonary blastoma, metastatic carcinoma of unknown primary, primary pulmonary lymphoepithelioma-like carcinoma, and benign neoplasms of the lung. In some embodiments, the cancer is NSCLC.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

In another aspect, provided herein are methods for assisting in the prevention and/or treatment of a condition in a subject in need thereof, the methods including the steps of administering to the subject a first therapy including one or more constructs, recombinant nucleic acids, engineered cells, or pharmaceutical compositions as disclosed herein, and administering to the subject at least one additional therapies, wherein the first therapy and at least one additional therapies together prevent and/or treat the condition in the subject. In some embodiments, the methods include administering to the subject a first therapy including an effective number of the engineered cells as disclosed herein, wherein the engineered cells treat the condition.

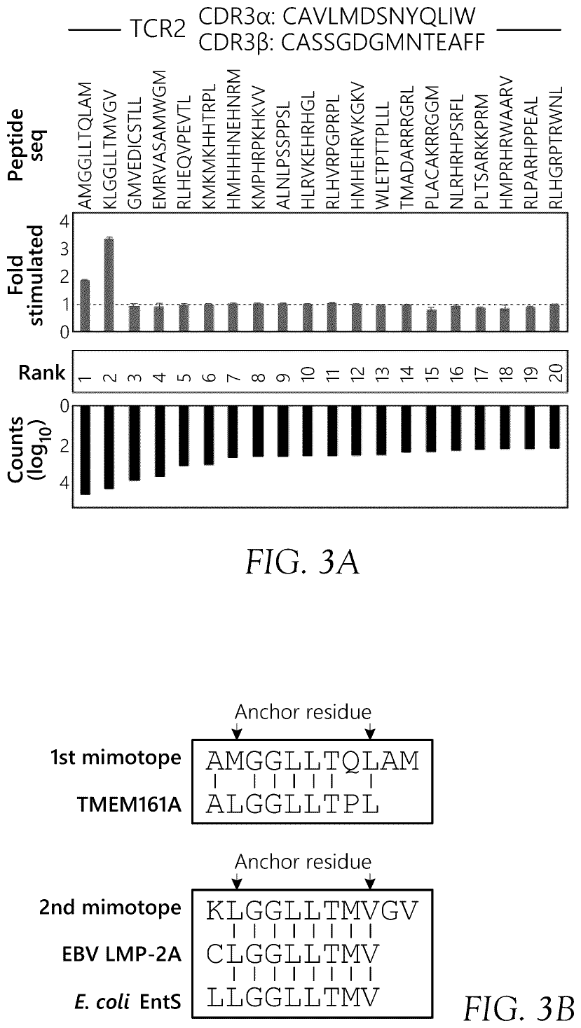

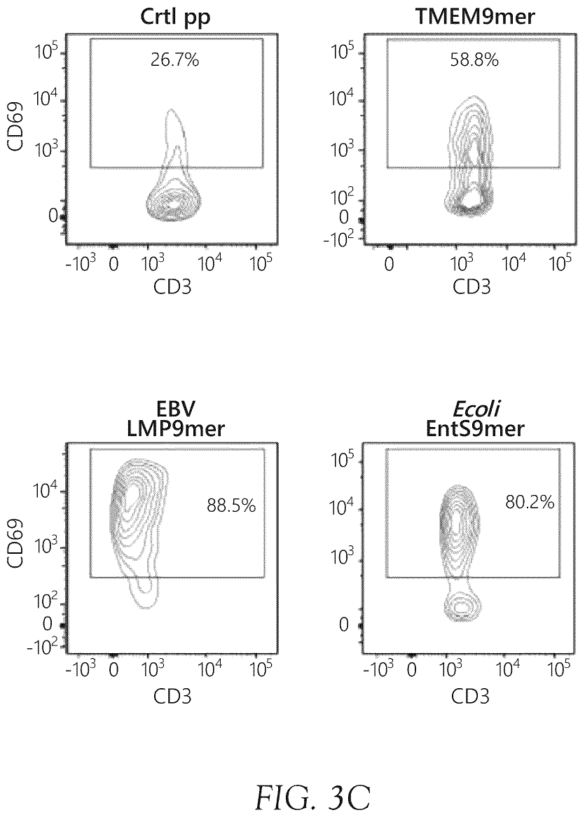

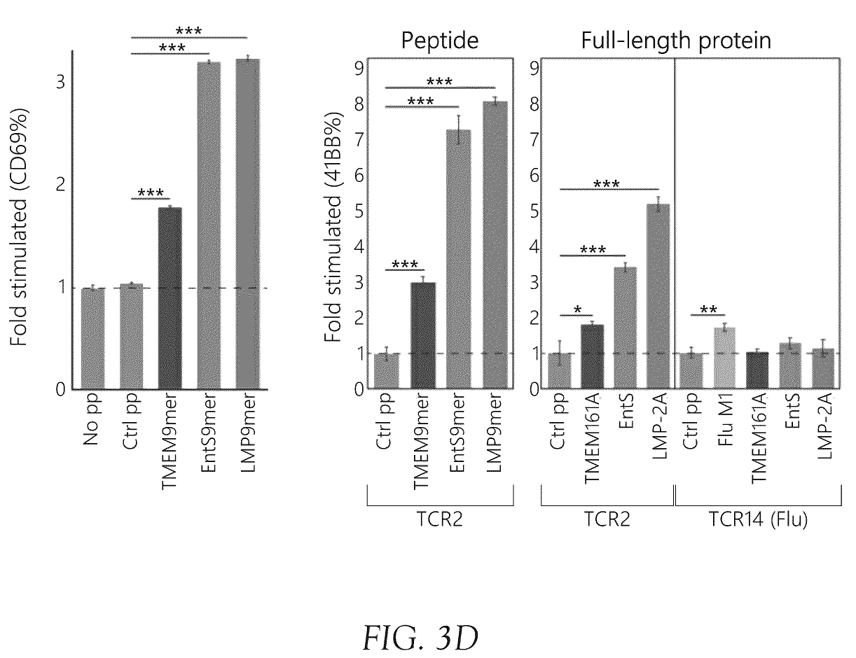

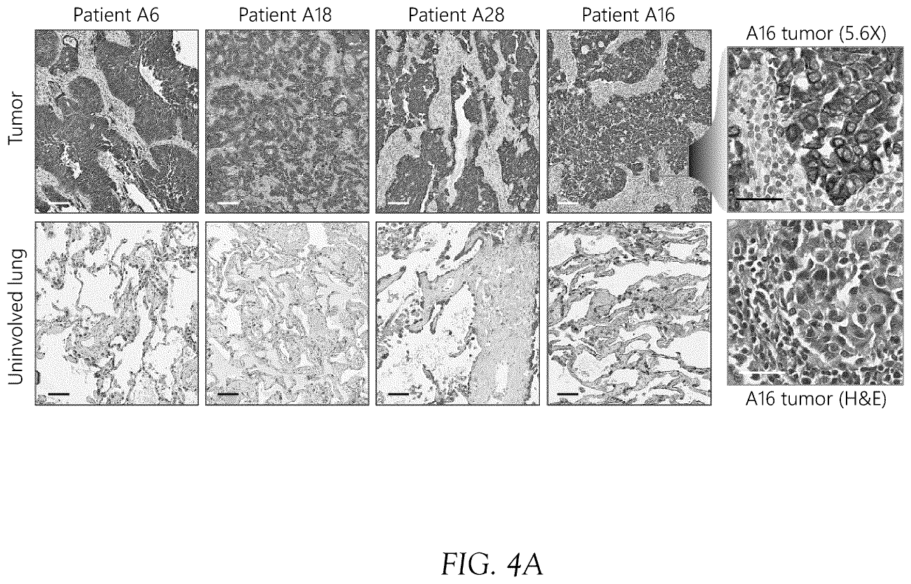

As described in greater detail below, e.g., Example 29 and FIG. 3, various constructs of the disclosure are capable of binding antigens derived from viral and bacterial pathogens. In some embodiments of the disclosure, provided herein are methods for the diagnosis, prevention, and/or treatment of a malignancy associated with a microbial infection. In some embodiments, the malignancy is associated with a bacterial infection. Non-limiting examples of malignancies associated with a bacterial infection include colon cancer associated with Streptococcus bovis infection and stomach cancer associated with Helicobacter pylori infection. In some embodiments, the bacterial infection is an Escherichia coli infection. Examples of common E. coli infections include cholecystitis, bacteremia, cholangitis, urinary tract infection (UTI), and traveler's diarrhea, and other clinical infections such as neonatal meningitis and pneumonia. Additional illnesses associate with E. coli include bloody diarrhea (hemorrhagic colitis), nonbloody diarrhea, the hemolytic uremic syndrome, and thrombotic thrombocytopenic purpura.

In some embodiments, provided herein are methods for the diagnosis, prevention, and/or treatment of a malignancy associated with a viral infection. In some embodiments, the malignancy associated with an infection by Epstein-Barr virus (EBV), which was originally discovered through its association with Burkitt lymphoma, but has since been linked to a remarkably wide range of lymphoproliferative lesions and malignant lymphomas of B-, T- and NK-cell origin. Examples of EBV-associated malignancies that can suitably be diagnosed, prevented, and/or treated by using the compositions and methods disclosed herein include Hodgkin lymphoma, Burkitt lymphoma, diffuse large B cell lymphoma, nasopharyngeal carcinoma, gastric carcinoma, post-transplant lymphoproliferative disease, B lymphoproliferative disease. Additional EBV-associated malignancies that can suitably be diagnosed, prevented, and/or treated by using the compositions and methods disclosed herein include, but are not limited to, T-cell lymphoproliferative disease, NK-cell lymphoproliferative disease, NK-cell lymphomas, T-cell lymphomas, NK-cell lymphomas, T-cell leukemias, leiomyosarcomas, and lymphoepithelioma-like carcinomas.

### Additional Therapies

As discussed above, some embodiments of the disclosure provide methods for the prevention or treatment of a condition in a subject, wherein the methods include administering a composition as disclosed herein to the subject as a single therapy (e.g., monotherapy). In addition, in some embodiments of the disclosure, the composition is administered to the subject individually as a first therapy or in combination with at least one additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. In some embodiments, the first therapy and the at least one additional therapies are administered concomitantly. In some embodiments, the first therapy is administered at the same time as the at least one additional therapies. In some embodiments, the first therapy and the at least one additional therapies are administered sequentially. In some embodiments, the first therapy is administered before the at least one additional therapies. In some embodiments, the first therapy is administered after the at least one additional therapies. In some embodiments, the first therapy is administered before and/or after the at least one additional therapies. In some embodiments, the first therapy and the at least one additional therapies are administered in rotation. In some embodiments, the first therapy and the at least one additional therapies are administered together in a single formulation.

In yet another aspects, provided herein are various methods for obtaining a construct as disclosed herein, the methods include (a) identifying a plurality of TCRs associated with a health condition; (b) determining a sequence of a CDR3β present in each of the identified TCRs; (c) identifying one or more cognate antigens commonly recognized by the CDR3β sequences; (c) making a construct including a CDR3β sequence determined in (b), wherein the construct is capable of binding to the one or more cognate antigens. In some embodiments, the condition is a proliferative disease. In some embodiments, the proliferative disease is a cancer. In some embodiments, the cancer is a lung cancer. In some embodiments, the condition is a malignancy associated with a bacterial infection or viral infection. In some embodiments, the condition is a malignancy associated with an infection by Epstein-Barr virus (EBV) or Escherichia coli.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the constructs, recombinant nucleic acids, engineered cells, or pharmaceutical compositions as provided and described herein, as well as written instructions for making and using the same.

In some embodiments, the kits of the disclosure further include one or more means useful for the administration of any one of the provided constructs, recombinant nucleic acids, engineered cells, or pharmaceutical compositions to an individual. For example, in some embodiments, the kits of the disclosure further include one or more syringes (including pre-filled syringes) and/or catheters (including pre-filled syringes) used to administer any one of the provided constructs, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative control constructs, positive control constructs, reagents suitable for in vitro production of the constructs.

In some embodiments, the components of a kit can be in separate containers. In some other embodiments, the components of a kit can be combined in a single container.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Example 1

### Clinical Samples

Protocols for collection of human tissue and blood were approved by the Stanford Institutional Review Board (IRB 15166 and IRB 21319). Inclusion criteria included adult patients (age >=18 years), known or suspected diagnosis of NSCLC, primary tumor >2 cm, and consent for research. Patients receiving neoadjuvant therapy or patients with underlying lung infection, inflammatory, or fibrotic disease were excluded. Overall 21 patients with surgically-resectable NSCLC treated at Stanford were included in this study. DNA was extracted from peripheral blood PBMC (Qiagen) for HLA tying.

### Example 2

### Tissue Processing

Tissue was processed within 2 hours from surgery. Tissue was divided and one section for cell suspensions and another section for histology. Cell suspensions were generated by mincing of tissue followed by digestion with collagenase III (200 IU/mL) and DNAse (100 U/mL) (Worthington Biochemical) for 40 minutes in RPMI and passing through a 70-um filter. Sections for histology were fixed in 4% paraformaldehyde and transferred to 70% ethanol solution the following day.

### Example 3

### FACS Analyses

T cells were isolated from tumor single cell suspensions by antibody staining followed by cell sorting on a 5-laser FACSAria Fusion (Stanford FACS Facility) purchased using funds from the Parker Institute for Cancer Immunotherapy. Tumor cell suspensions were stained in PBS with Zombie Aqua dye (Biolegend) for viability assessment. This was followed by staining in PBS with 2% FBS in Fc Blocking solution (Biolegend) plus the following antibodies: anti-CD4 (OKT4, Biolegend), anti-CD8 (SKI, Biolegend), anti-CD3 (OKT3, Biolegend), anti-CD45 (H130, Biolegend), anti-CD25 (BC96, Biolegend), anti-PD-1(EH12.2H7, Biolegend), anti-CD137 (4B4-1, BD Biosciences), anti-HLA-DR (L243, Biolegend). CD3+CD45+AquaZombie− cells were index sorted directly into 96-well plates preloaded with 4 uL of capture buffer, snap frozen on dry ice, and stored at −80° C.

### Example 4

### GLIPH2 Analyses and Establishment of T Cell Specificity Groups

The GLIPH2 algorithm was implemented for the establishment of T cell specificity groups using 778,938 distinct CDR3β sequences from the MD Anderson NSCLC dataset (Reuben, A., et al., Nat Commun, 2020. 11(1): p. 603). Briefly, by comparing with the reference dataset of 273,920 distinct CDR3β sequences (both CD4 and CD8) from 12 healthy individuals, GLIPH2 first discovered clusters of CDR3β sequences sharing either global or local motifs as previously described (Huang, H., et al., Nat Biotechnol, 2020. October; 38(10): 1194-1202). The output of CDR3β clusters with shared sequence motifs is accompanied by multiple statistical measurements to facilitate the calling of high-confidence specificity groups, including biases in Vβ gene usage, CDR3β length distribution (relevant only for local motifs), cluster size, HLA allele usage, and clonal expansion. To establish high-confidence specificity groups with the NSCLC dataset, TCR specificity groups with at least 3 distinct CDR3β members from a minimum of 2 different patients with significant biases in Vβ gene usage, and CDR3β clonal expansion in comparison with the reference dataset were prioritized. This led to the discovery of 4,300 specificity groups that formed the basis for further analyses throughout the study.

### Example 5

### Annotation of Specificity Groups with Tetramer-Derived CDR3β Sequences

To annotate inferred specificity groups from lung cancer patients, a combined GLIPH analysis using both the MD Anderson lung cancer patient CDR3β sequences and publicly available, tetramer-derived CDR3β sequences was performed (Glanville, J., et al., Nature, 2017. 547(7661): p. 94-98; Shugay, M., et al., Nucleic Acids Res, 2018. 46(D1): p. D419-D427; and Song, I., et al., Nat Struct Mol Biol, 2017. 24(4): p. 395-406). To do so, tetramer-derived CDR3β sequences that could form TCR specificity groups were first identified by running an independent GLIPH analysis with a total 10,051 CDR3β sequences from the tetramer datasets. This led to the formation of 395 specificity groups containing 1,561 CDR3β sequences. These 1,561 CDR3β sequences were then combined with the 778,938 CDR3β sequences from the MD Anderson lung cancer dataset for the aforementioned GLIPH2 analysis. Any specificity group that includes at least one CDR3β sequence from the tetramer data is considered “annotated” and would be assigned a specificity and HLA restriction according to the associated tetramer sequence(s). Of note, in all cases where multiple tetramer-derived CDR3β sequences were found in a given specificity group, there was only one dominant tetramer-defined specificity/HLA involved.

### Example 6

### In Silico Validation of HLA Restriction Inference

For the tetramer-annotated specificity groups mentioned above (n=72), the inferences of HLA restriction made by the GLIPH2 algorithm against the HLA restriction informed by tetramers was validated. Specificity groups annotated with HLA-A*02 (n=50 out of 72) or HLA-B*08 (n=8 out of 72) tetramers were chosen for the validation because they were the most prevalent. To validate a specificity group for enrichment with HLA-A*02 alleles, a contingency table was first constructed with the number of total patients in the specificity group (query patients), number of query patients carrying HLA-A*02 supertype allele(s) (Huang, H., et al., Nat Biotechnol, 2020. October; 38(10): 1194-1202), number of all NSCLC patients (n=178), and number of all NSCLC patients carrying HLA-A*02 supertype allele(s) (n=79). p values were then calculated by using the hypergeometric test (phyper in R). The numbers of specificity groups significantly enriched with HLA-A*02 supertype alleles (p<0.05 by the hypergeometric test) were reported as a fraction over the number of specific groups annotated with HLA-A*02 tetramers (n=18 out of 50). The numbers of specificity groups significantly enriched with HLA-A*02 supertype alleles were also reported as a fraction over the number of specificity groups annotated with non-HLA-A*02 tetramers (n=0 out of 22). This process for the validation of specificity groups enriched was repeated with HLA-B*08 supertype alleles.

### Example 7

### Bootstrapping of HLA-A*02:01 Specificity Group Quantifications

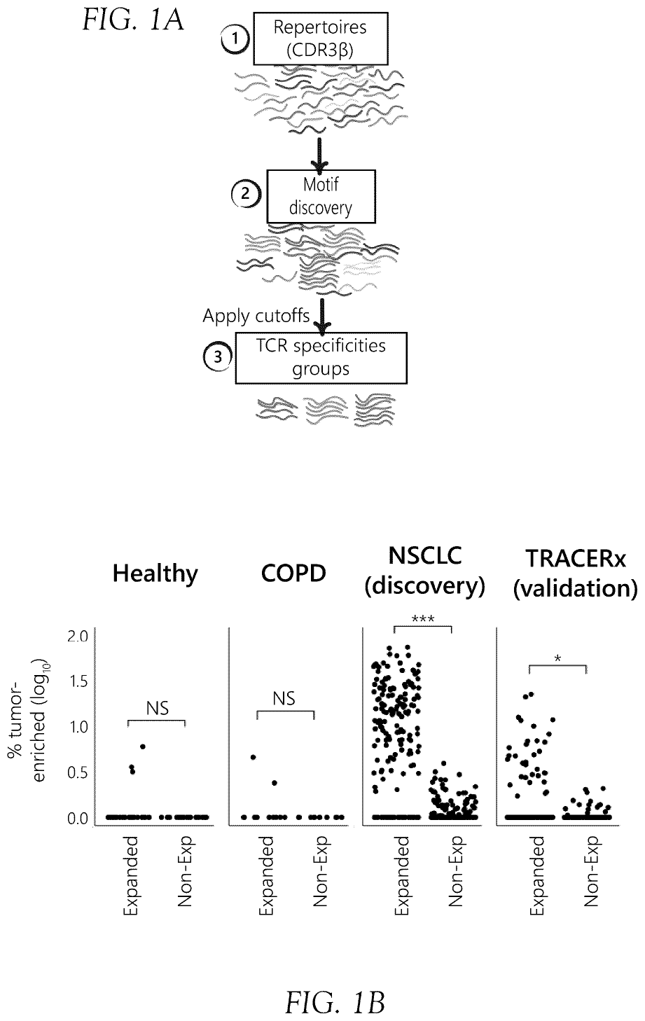

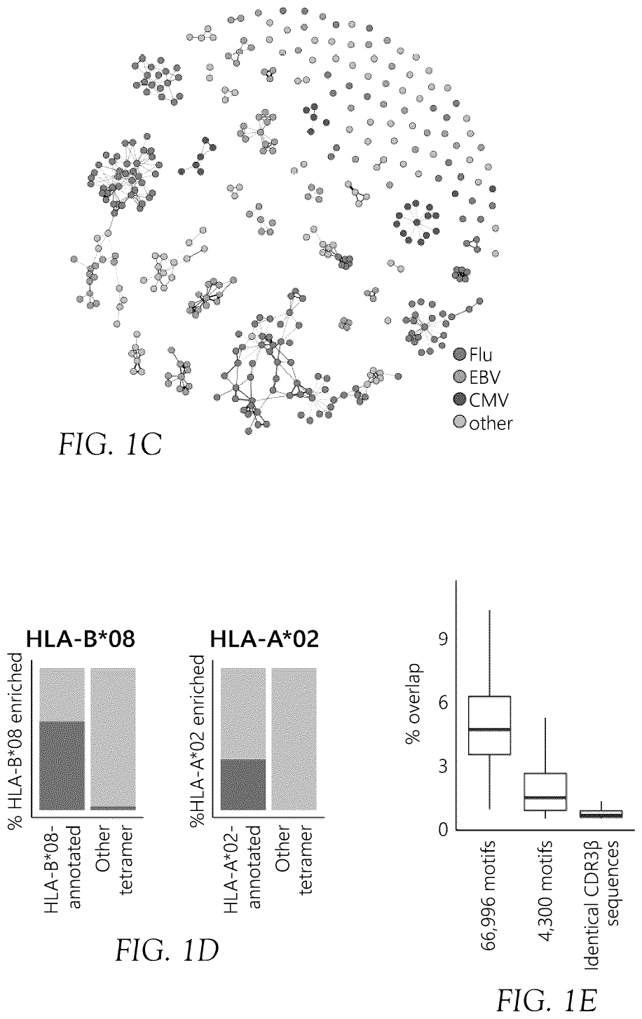

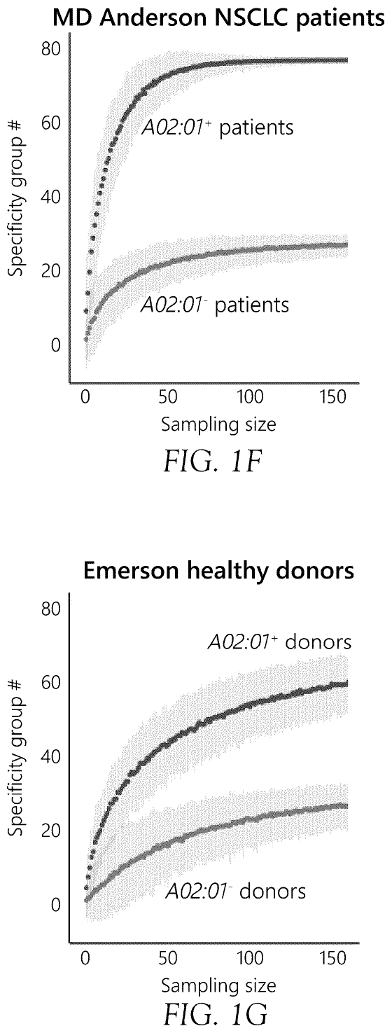

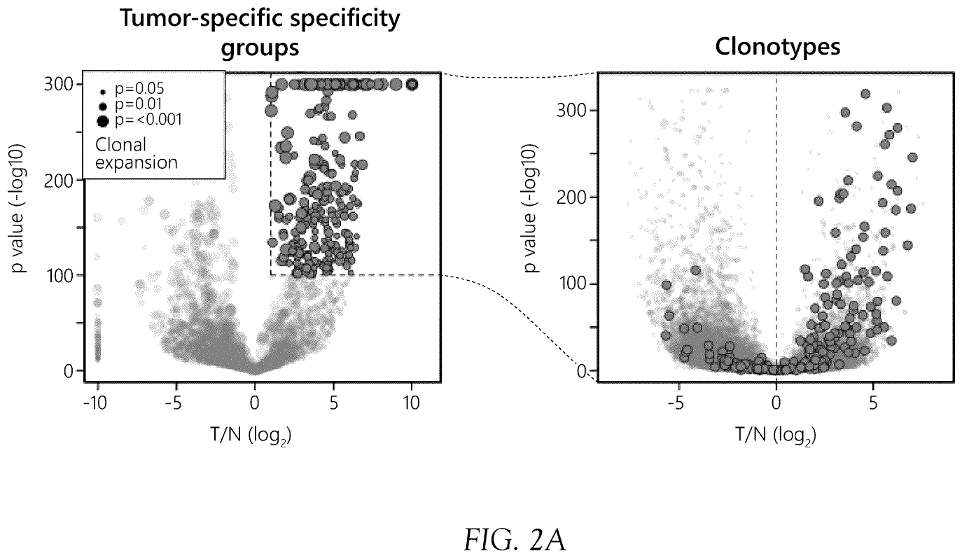

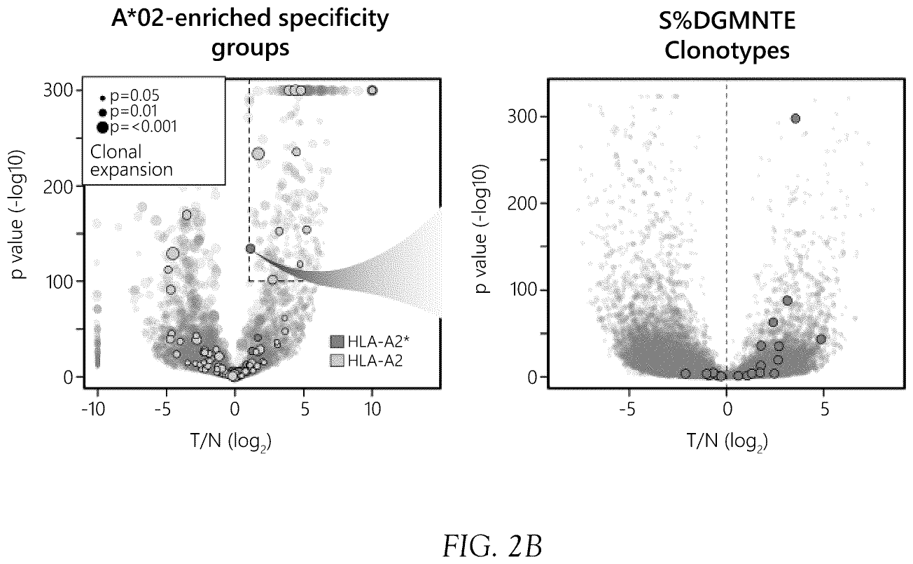

To estimate the number of HLA-A*02:01+ NSCLC patients needed to cover 50% of all HLA-A*02:01-enriched specificity groups (n=77), a bootstrapping process was performed through random sampling of patients with incremental sampling sizes. First, 77 specificity groups (from the 4,300 NSCLC-enriched specificity groups), which were significantly enriched with the HLA-A*02:01 allele (p<0.05), were first established. Bootstrapping was conducted with random sampling (with replacement) of 1 through 160 patients for 100 times. For each sampling event, the sum of HLA-A*02:01-enriched specificity groups found was tallied using the CDR3β sequences from the sampled patients (specificity count, FIGS. 1F and 1G). The mean and the standard error of the specificity counts from the bootstrapping process were then calculated. As an internal control, we repeated the bootstrapping process on the rest of HLA-A*02:01−NSCLC patients. To compare with specificity groups from a healthy cohort, a total of 989,816 distinct CDR3β sequences from 304 HLA-A*02:01+ and 1,153,600 CDR3β sequences from 362 HLA-A*02-healthy donors' PBMC from a publicly available dataset (Emerson dataset, Emerson, R. O., et al., Nat Genet, 2017. 49(5): p. 659-665) were used. To adjust for the differences in sequencing depth (below), 5000 CDR3β sequences (with the highest frequencies) from each healthy donor were included for the GLIPH analysis. To address the influence of clonal expansion on specificity group quantification, the bootstrapping results were compared with the aforementioned HLA-A*02:01-enriched specificity groups to an equal number of HLA-A*02:01-enriched specificity groups without clonal expansion (n=77). A similar strategy was used to address how the total number of specificity groups impacted this result. Bootstrapping was performed using various enrichment cutoffs for HLA-A*02 enrichment (p <0.05, n=1,267; p<0.025, n=319; p<0.01, n=72 specificity groups). Finally, to address the impact of sequencing depth on specificity group quantification, the total input CDR3β sequences was down-sampled randomly in the bootstrapping process by the indicated proportions (50%, 25%, 12.5%, or 0% down-sampled).

### Example 8

### GSEA Analysis of the TCGA Data

Normalized gene expression data from bulk RNA-Seq analyses of human NSCLC resected tumors and uninvolved lungs from the Cancer Genome Atlas (TCGA) were downloaded from the NCI GDC Legacy Archive (n=1,017 for tumors and n=110 for uninvolved lungs). To conduct geneset enrichment analysis (GSEA) with the TCGA dataset, the correlation coefficients between any gene and TMEM161A was first calculated using the Pearson correlation. The sorted gene list based on the correlation coefficient with TMEM161A gene expression was then used for GSEA with the Preranked tool (v2.2.2, Broad Institute) and all hallmark genesets. The signature scores were derived using the gene lists of indicated hallmark signatures with the single-sample GSEA method as described previously (ssGSEA, Abazeed, M. E., et al., Cancer Res, 2013. 73(20): p. 6289-98; and Barbie, D. A., et al., Nature, 2009. 462(7269): p. 108-12).

### Example 9

### FACS Sorting of HLA-A*02 Tetramer+CD8 T Cells

Recombinant HLA-A*02 monomer with UV exchangeable peptide were either synthesized as previously described (Altman, J. D. and M. M. Davis, Curr Protoc Immunol, 2003. Chapter 17: p. Unit 17 3) or purchased commercially (Biolegend). UV peptide exchange was performed over 20 minutes with 1 mM of peptide in PBS using Strategene UV Stratalinker 2400. Streptavidin conjugated fluorophore was added incrementally the following day for a final 4:1 molar ratio of MHC:streptavidin. Tetramer staining was performed in PBS plus 2% FBS in Fc Blocking solution (Biolegend) at room temperature for 1 hour. For peripheral blood samples, cells were subsequently stained with anti-TCRγδ (B1, Biolegend), anti-CD19 (H1B19, Biolegend), anti-CD14 (M5E2, Biolegend), anti-CD3 (OKT3, Biolegend), anti-CD4 (RPA-T4, Biolegend), anti-CD8 (HIT8a, Biolegend), and live/dead near-IR dye (Invitrogen). For tumor samples, cells were stained with anti-CD4 (OKT4, Biolegend), anti-CD8 (HIT8a, Biolegend), anti-CD3 (UCHT1, Biolegend), anti-CD45 (H130, Biolegend).

### Example 10

### Single-Cell RNA-Seq (scRNA-Seq) Sample Preparation with the Smart-Seq2 Method

Full transcriptomes from FACS sorted T cells at the single-cell level were generated according to the previously reported procedures with some modifications (Picelli, S., et al., Nat Protoc, 2014. 9(1): p. 171-81). First strand cDNA was then generated with Takara's SMARTScribe Reverse Transcriptase kit according to manufacturer's protocol (Takara Bio). Notable changes from the previously reported Smart-Seq2 RT step includes: 2 mM of dNTP and 2 μM of oligo-dT were included in the capture buffer; 1M of Betaine and additional 6 mM MgCl₂ were included in the RT reaction buffer. The cDNA samples were then amplified with the KAPA Library Quantification kit for 22-25 cycles (Roche). One μL of amplified cDNA (of total 25/well) was used for single-cell TCR-sequencing and thus bypassing the RT step as reported previously (Han, A., et al., Nat Biotechnol, 2014. 32(7): p. 684-92). To proceed with scRNA-Seq, full-length cDNA samples were first cleaned up with 0.6-0.8× volume of pre-calibrated AMPure XP beads (Beckman Coulter) to exclude DNA fragments smaller than 500 base pairs. The automatic liquid handler Biomek FXP Automated Workstation (Beckman Coulter) was used in order to eliminate cell-to-cell variabilities. The quality of purified full-length cDNA was validated with the AATI Fragment Analyzer (Agilent). Subsequently, the measurements from the Fragment Analyzer were used in order to normalize the cDNA input with a Mantis liquid handler (Formulatrix). The cDNA samples were then consolidated into a 384-well plate (LVSD) with a Mosquito X1 liquid handler (TTP labtech). After transfer, Illumina sequencing libraries were prepared using a Mosquito HTS liquid handler (TTP labtech). Only 0.4 uL (of total 23 uL) of cDNA per well were used to make the full transcriptome libraries with the Nextera XT DNA Library Preparation Kit (Illumina, FC-131-1096). Custom-made i5 and i7 unique 8-bp indexing primers (IDT) were used to multiplex 384 wells in a single sequencing run. The libraries were amplified on a C1000 Touch™ Thermal Cycler with 384-Well Reaction Module (Bio-rad). The pooled libraries were checked with the Agilent 2100 Bioanalyzer (Stanford PAN facility) and acquired paired-end sequences (150 bp×2) on a Hiseq 4000 Sequencing System (Illumina) purchased with funds from NIH (S10OD018220) for the Stanford Functional Genomics Facility (SFGF).

### Example 11

### Single-Cell Sequencing of the TCRα/β Chains

Single T cells were sorted and captured as described above in the method for scRNA-Seq sample preparation. Following first strand cDNA synthesis (Takara) and amplification (Roche), one microliter of amplified cDNA (of total 25 uL/well) was used for single-cell TCR-sequencing and thus bypassing the RT step as reported previously (Han, A., et al., Nat Biotechnol, 2014. 32(7): p. 684-92). Nested PCR was performed with TCRα/β primers carrying multiplexing barcodes that enabled pooled CDR3α/β sequencing in a single Miseq run. Paired sequencing reads were joined, demultiplexed, and mapped to the human TCR references from the international ImMunoGeneTics information System® (IMGT) with custom scripts as reported previously (Han, A., et al., Nat Biotechnol, 2014. 32(7): p. 684-92).

### Example 12

### Data Analyses of scRNA-Seq Results

Sequencing reads were first de-multiplexed and binned into separate fastq files that correspond with the full transcriptomes of individual T cells. STAR aligner (2.7.1a) was used to map the reads with default parameters against human genome reference GRCh38 (v21) from the UCSC genome browser. Mapped reads were sorted and indexed with samtools (1.4). Gene expression was first quantified by counting reads mapped to genes with htseq-count (HTSeq 0.9.1) using the following settings: --stranded=no --type=exon --idattr=gene name --mode=intersection-nonempty. Unless otherwise stated, all single-cell T cell states were analyzed with Seurat (3.1.4) packages in R using raw read counts. To derive TCR repertoires from the scRNA-Seq results, reads mapped to both the TCRα and TCRβ genes were first reconstructed with the TraCeR algorithm as described previously (Stubbington, M. J. T., et al., Nat Methods, 2016. 13(4): p. 329-332). The reconstructed DNA sequences were then submitted to the IMGT to call gene segment usage and the CDR3 amino acid sequences through HighV-QUEST.

### Example 13

### GLIPH2 Analysis on the CDR3b Sequences from the TRACERx NSCLC Cohort

Raw fastq files (n=202) of the bulk CDR3β nucleotide sequences from the TRACERx cohort of NSCLC were downloaded from the Short Read Archive as reported (Joshi, K., et al., Nat Med, 2019. 25(10): p. 1549-1559). The amino acid sequences of CDR3β, V gene usage, and clonal counts were subsequently derived by using the custom pipeline established previously (Han, A., et al., 2014 supra). To quantify the percentages of tumor-enriched specificity groups shown in FIG. 1C, joint GLIPH2 analyses were first conducted with combined CDR3β sequences from the MD Anderson cohort (n=778,938) and the bulk CDR3β sequences from each tumor sample of the TRACERx cohort. The total percentages (%) of top-20 clonally expanded as well as the rest CDR3β clonotypes that belonged to the 449 tumor-enriched specificity groups were then derived for each tumor (n=202).

### Example 14

### Soluble Biotinylated TCRα/β Chains for Yeast Screen

Soluble TCRα/β chains used for yeast selections were made as described previously (Gee, M. H., et al., Cell, 2018. 172(3): p. 549-563 e16). Briefly, synthetic gene blocks (gBlocks®) of N-terminal truncated TCRα or TCRβ chain V and modified C gene fragments were assembled into the baculoviral pAcGP67a construct (BD Biosciences) with Gibson assembly (New England BioLabs). The final baculoviral plasmid was co-transfected into SF9 cells (ATCC) with Bestbac 2.0 (Expression systems) with FuGENE® 6 (Promega) to make the crude viral supernatant (P0). Subsequently, viruses were passaged at a dilution of 1:500 in 30-50 mL cultures at a density of 1×10⁶ cells/mL to generate higher titer viruses (P1). To generate the soluble TCRα/β chains, up to 4 liters of High Five (Hi5, ThermoFisher Scientific) cells were infected with P1 baculovirus at a dilution of 1:500-1:1000 at a density of 2×10⁶ cells/mL for a week before protein purification. Recombinant TCRα/β chains were bound with Ni-NTA resin (QIAGEN) in the Hi5 cell media for 3 hours at room temperature, washed with 20 mM imidazole in 1×HBS at pH 7.2, and eluded eluted in 200 mM imidazole in 1×HBS at pH 7.2. After buffer exchange to 1×HBS at pH 7.2 with a 30 kDa filter (Millipore), purified proteins were biotinylated overnight with birA ligase in the presence of 100 μM biotin, 40 mM Bicine at pH 8.3, 10 mM ATP, and 10 mM Magnesium Acetate at 4° C. Biotinylated proteins were purified by size-exclusion chromatography using an AKTAPurifier Superdex 200 column (GE Healthcare) and validated on a SDS-PAGE gel to confirm the stoichiometry and biotinylation with excess streptavidin.

### Example 15

### Identification of Novel T Cell Antigens with HLA-A02 Yeast Libraries

To uncover the cognate antigens of the candidate TCRα/β, the yeast HLA-A*02 libraries displaying highly diverse peptides of 4 different length were used (Gee, M. H., et al., 2018 supra). Briefly, four separate naïve HLA-A*02 libraries carrying distinct lengths of peptides were first expanded to beyond 10× diversities in SDCAA pH 6.0 before induction of the peptide-HLA-Aga2p composite proteins with SGCAA. Induced libraries were used for affinity-based selection with biotinylated soluble TCRα/β chains coupled to streptavidin-coated magnetic MACS beads (Miltenyi) in the presence of 0.5% bovine serum albumin and 1 mM EDTA to reduce the background. The selected yeast clones in SDCAA were cultured until confluency, then induced confluent cells in SGCAA for 2-3 days before the next round of selection. The selection was repeated four times and then enrichment of cognate antigens was confirmed with Sanger sequencing of 20 colonies. Once confirmed, the plasmid DNA from 5-10×10⁷ yeast cells per round of selection was prepared by miniprep (Zymoprep II kit, Zymo Research). The peptide coding regions were PCR-amplified with composite oligos with Illumina P5/P7-Truseq indexed adapters and gel purified for pooled sequencing on a Miseq sequencer (2×150 V2 kit)

### Example 16

### Lentiviral TCR Transduction

TCRα chain, P2A linker, and TCRβ chain fusion gene fragments were purchased from IDT and cloned into MCS of the EF1a-MCS-GFP-PGK-puro lentiviral vector (Glanville, J., et al., Nature, 2017. 547(7661): p. 94-98). HEK-293T cells were plated on a 10-cm dish at a density of 7.5×10⁶ cells in 10 mL of DMEM the day prior to transfection. 293 Ts were co-transfected with 3.3 μg of the lentiviral plasmid, 2.5 μg of the gag-pol plasmid, and 0.83 μg of the VSV-G envelope plasmid pre-mixed with 33 μL of PEI in 120 μL of Opti-MEM (ThermoFisher Scientific). After 24 hours, the medium was replenished and viral supernatant was collected 24 and 48 hours later. TCR-deficient Jurkat cells (below) were transduced with viral supernatant, TCR expression was assessed by flow cytometry, and TCR-expressing cells were sorted based on the expression of GFP, CD3, and the transduced TCRα/β chains. For lentivirus expressing full-length EntS, LMP2, and FluM1, gene fragments were also purchased from IDT and cloned into MCS of EF1a-MCS-GFP-PGK-puro lentiviral vector. Lentivirus for expressing human TMEM161A (NM 017814) was purchased from GeneCopoeia. Lentivirus was produced as described above, and 293T cells stably expressing HLA-A*02 (293A2) were transduced with viral supernatant. Transduced 293A2 cells were sorted based on GFP expression and used for in vitro T cell stimulation.

### Example 17

### Retroviral TCR Transduction

For retroviral-mediated expression of TCR2 in primary T cells, TCRα chain, P2A linker, and TCRβ chain were PCR amplified from the lentiviral vector (described above) and cloned into the MCS of an MSGV1-based retroviral vector (gift from Steve Rosenberg laboratory) using In-Fusion Cloning (Takara). For retroviral-mediated expression of TCR14 in primary T cells, TCRα chain, P2A linker, and TCRβ chain fusion gene fragments were purchased from IDT and cloned into MCS of an MSGV1-based retroviral vector.

### Example 18

### Cell Cultures

The Jurkat 76 T-cell line deficient for both TCRα and TCRβ were provided by Dr. Shao-An Xue (Department of Immunology, University of College London). Jurkat cells and primary T cells were grown in complete RPMI (ThermoFisher) containing 10% FBS, 25 mM HEPES, 290 μg/mL L-glutamine, 100 U/mL penicillin, 100 U/mL streptomycin, 1 mM sodium pyruvate, and 1× non-essential amino acids. T2 cells were grown in IMDM (Fisher Scientific) with 20% FBS, 290 μg/mL L-glutamine, 100 U/mL penicillin, 100 U/mL streptomycin. 293T cells stably expressing HLA-A*02 were provided by Dr. Steve Feldman (Stanford School of Medicine) and grown in DMEM (ThermoFisher) with 10% FBS, 290 g/mL L-glutamine, 100 U/mL penicillin, 100 U/mL streptomycin.

### Example 19

### In Vitro Stimulation of the Jurkat T Cell Clones

Jurkat 76 cells expressing the exogenous TCR of interest were sorted and co-cultured with T2 cells in complete RPMI as detailed above. Peptides were dissolved in DMSO at 20 mM stock concentration and diluted to a final concentration of 2 mM. After 18 hours of stimulation, cells were washed and stained with anti-CD3 (OKT3, Biolegend), anti-CD69 (FN50, Biolegend), and anti-TCRα/β (IP26, Biolegend) antibodies. Cells were acquired using FACS Fortessa (BD Biosciences) automated high throughput sampler, and data analyzed using FlowJo software (Treestar).

### Example 20

### In Vitro Stimulation of Primary T Cells Expressing Candidate TCRα/β

T cells were isolated from a leukoreduction system chamber from an HLA-A*02 positive healthy donor from the Stanford institutional blood bank using the RosetteSep human T cell enrichment cocktail (Stem Cell Technologies) and viably stored in liquid nitrogen. For T cell activation, T cells were thawed and stimulated with anti-CD3/CD28 beads (Life Technologies) in the presence of IL-2 (100 IU/mL). On days 1 and 2, activated T cells were retrovirally transduced using Retronectin (Takara) coated plates in media containing 100 IU/mL IL-2. Anti-CD3/CD28 beads were removed on day 3 and media containing IL-2 were replenished once every 2 days. Following 8 days of in vitro expansion, T cells were co-cultured with 293A2 cells expressing full-length TMEM161A, EntS, LMP2, FluM1, or GFP alone at a 1:1 ratio. Following 18 hours incubation, cells were stained with anti-CD3 (OKT3, Biolegend), anti-CD69 (FN50, Biolegend), anti-TCRα/β (IP26, Biolegend), anti-CD137 (4B4-1, BD Biosciences), and live/dead near-IR dye (Invitrogen). Data were acquired using FACS Fortessa (BD Biosciences) automated high throughput sampler, and analyzed using FlowJo software (Treestar).

### Example 21

### Immunohistochemistry of TMEM161A Expression in Multiple Human Cancers

Additional experiments were performed to demonstrate that multiple human cancers express TMEM161A protein. In these experiments, a tissue microarray consisting of over 100 human cancer tissues and normal tissues from paraffin-embedded sections were stained anti-TMEM161A antibody. Tissues were manually scored based on percent positivity and intensity for determination of H scores.

TMEM161A staining of paraffin-embedded tissue was performed according to standard procedures by the Stanford Human Pathology/Histology Service Center. Anti-TMEM161A antibody was stained at 1:50 (abcam ab180954), followed by HRP-conjugated secondary antibody. Tissue was counterstained with hematoxylin. Automated imaging analysis was performed using ImageJ (Rueden, C. T., et al., Bioinformatics, 2017. 18(1): p. 529) along with Fiji imaging processing package (Schindelin, J., et al., Nat Methods, 2012. 9(7): p. 676-82).

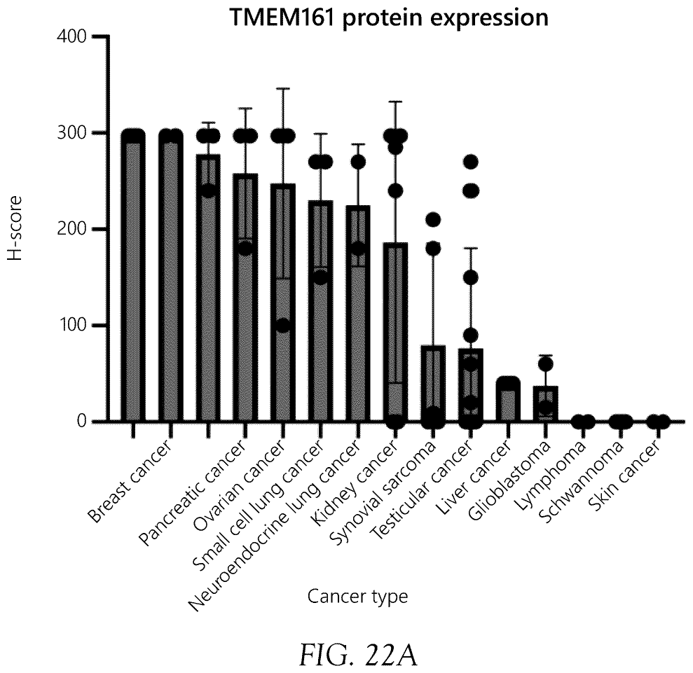

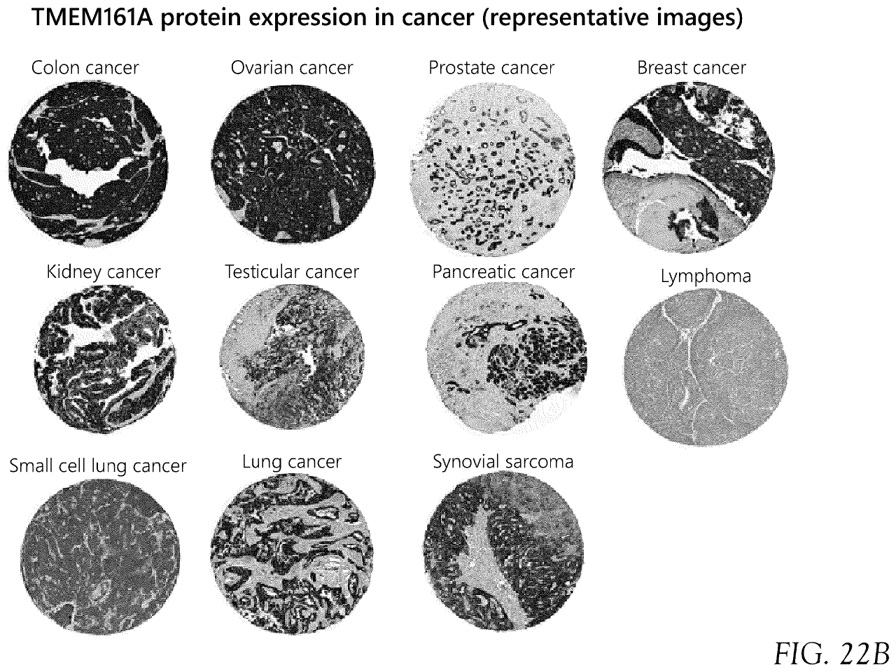

As shown in FIGS. 22A-22B, it was observed that multiple human cancers expressed TMEM161A protein. High levels of TMEM161A expressed were observed in colon cancer, breast cancer, kidney cancer, lung cancer, ovarian cancer, pancreatic cancer, prostate cancer, sarcoma, neuroendocrine cancer, and testicular cancer. Representative examples of TMEM161A expression in cancer tissue are shown, as well as quantification of TMEM161A expression by H-score.

### Example 22

### Whole-Exome Sequencing

Whole-exome sequencing of tumor DNA and matched germline leukocyte DNA was performed by inputting 75 ng of sheared genomic DNA for library preparation with the KAPA HyperPrep Kit (Roche) with modifications to the manufacturer's instruction, as described previously (Hellmann, M. D., et al., Clin Cancer Res, 2020). Library-prepared samples were captured with the SeqCap EZ MedExome Kit (NimbleGen) according to the manufacturer's instructions. Sequencing data were demultiplexed and mapped to hg19 using a custom bioinformatics pipeline, as described previously (Newman, A. M., et al., Nat Med, 2014. 20(5): p. 548-54). VarScan 2 (Koboldt, D. C., et al., Genome Res, 2012. 22(3): p. 568-76), Mutect (Cibulskis, K., et al., Nat Biotechnol, 2013. 31(3): p. 213-9), and Strelka (Saunders, C. T., et al., Bioinformatics, 2012. 28(14): p. 1811-7) were used to call variants use default parameters. Variants called by at least two of the approaches were then filtered by requiring: 1) variant allele frequency of at least 2.5%, 2) at least 30×depth in both tumor and germline samples, 3) zero germline reads, and 4) a population allele frequency of less than 0.1% in the Genome Aggregation database (Lek, M., et al., Nature, 2016. 536(7616): p. 285-91).

### Example 23

### Statistical Analysis

Unless stated otherwise, all statistical analyses performed in finding high-confidence specificity groups with GLIPH2 were Fisher's exact tests using the contingency tables with the CDR3β query set (specificity group) and the reference set (Huang, H., et al., 2020 supra). Poisson test was used to determine the representation bias in comparisons of distinct CDR3β sequences or specificity groups between tumors and uninvolved lungs. Student's t test was used to assess the results from all in vitro assays. Statistical significance was defined as p value <0.05.

### Example 24

### Establishing Specificity Groups from Tumor-Infiltrating T Cells in Human Lung Cancer

This Example describes the results of experiments performed to identify T cells recognizing shared tumor antigens in lung cancer.

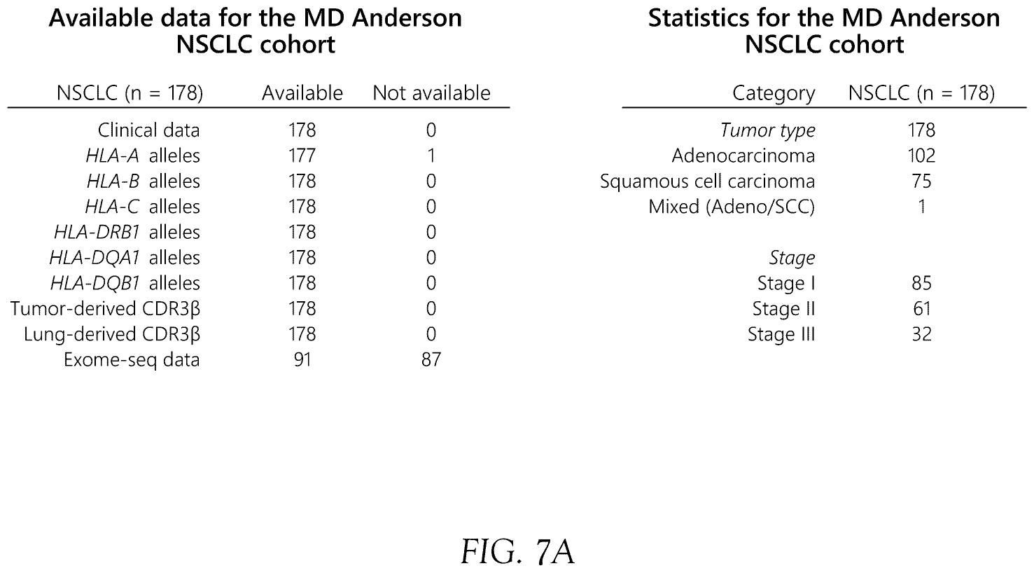

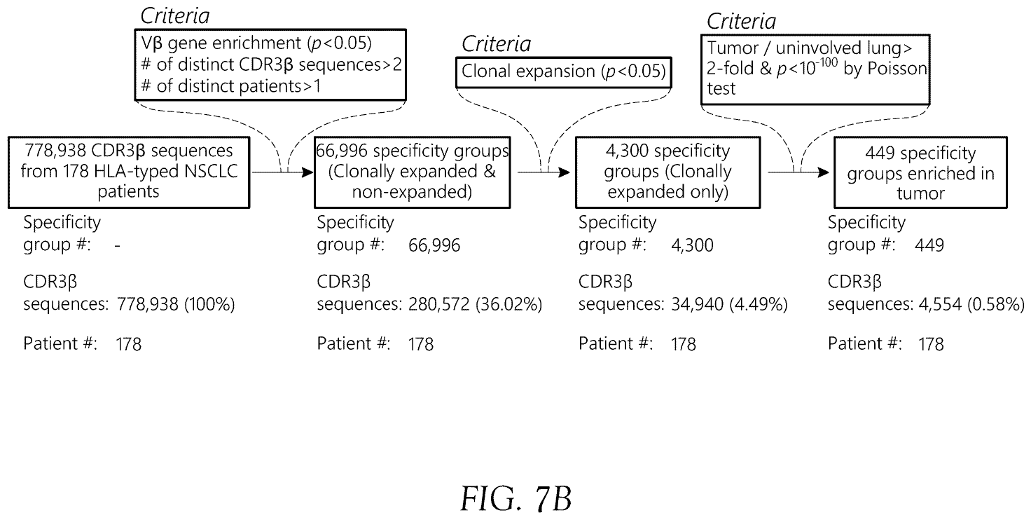

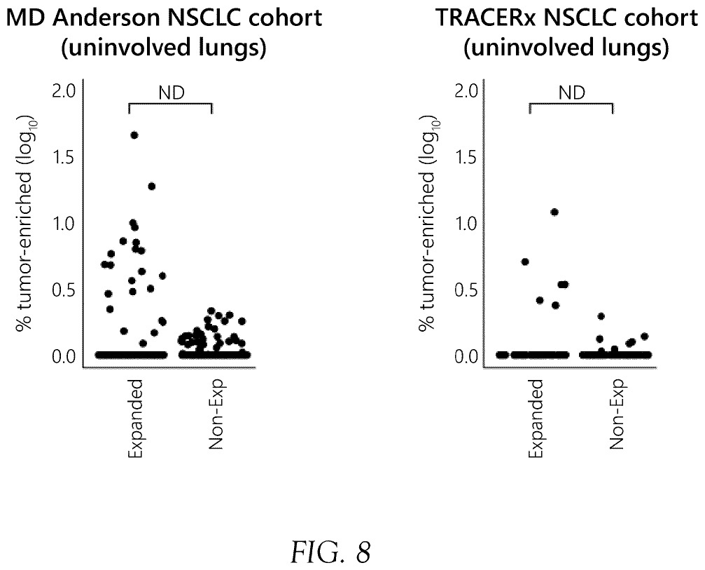

In order to identify T cells recognizing shared tumor antigens in lung cancer, specificity groups using GLIPH2 were established. As described previously, TCR clonotypes with a high probability of sharing specificities are grouped based on short amino acid sequence motifs embedded within the variable CDR3β regions of the TCR (Glanville, J., et al., 2017 supra). The improved GLIPH2 offers the advantage of analyzing large T cell repertoire datasets and identifying specificity groups carrying local or global sequence motifs with a much greater capacity (Huang, H., et al., 2020 supra). GLIPH2 algorithms were applied to a recently published T cell repertoire dataset of 778,938 distinct CDR3β sequences from 178 HLA-typed, non-small cell lung cancer (NSCLC) patients with surgically resectable disease (Reuben, A., et al., Nat Commun, 2020. 11(1): p. 603) (FIG. 7A). Of note, the T cell clonotypes from bulk sequencing were derived from both the surgically removed tumor as well as the uninvolved lung (FIG. 7A). With this dataset, 449 specificity groups enriched in tumors from NSCLC patients were established after applying a set of criteria including Vs gene enrichment and clonal expansion, the latter indicative of T cell antigen recognition (FIGS. 1A and 7B). To identify specificity groups related to antigens shared across these patients, a specificity group was further defined as including at least 3 distinct CDR3β sequences from a minimum of 2 patients. The fraction of clonotypes, which are members of the specificity groups as previously defined above, was established. It was found that significantly higher percentages of the most expanded TCR clonotypes in tumor belonged to the tumor-enriched specificity groups (FIG. 1B). In contrast, TCR clonotypes from patients' uninvolved lungs showed much lower percentages belonging to the tumor-enriched specificity groups (FIG. 8). It was next established that the 449 tumor-enriched specificity groups are relevant to lung cancer, and not merely to normal lung tissue or other types of lung disease. In a validation cohort of 7,363,492 clonotypes from 202 tumor samples representing 68 NSCLC patients (Joshi, K., et al., Nat Med, 2019. 25(10): p. 1549-1559), a significantly higher percentage of top expanded TCR clonotypes in tumor belonged to the 449 tumor-enriched specificity groups compared to the non-expanded counterparts (FIG. 1B). In contrast, a lower percentage of TCR clonotypes belonging to the tumor-enriched specificity groups in lung tissue from healthy donors and patients with COPD (without a cancer diagnosis) (Reuben, A., et al., 2020 supra) was observed, regardless of clonal expansion (FIG. 1B). In summary, the experimental data described herein has identified a set of specificity groups predicted to recognize shared tumor antigens across NSCLC patients.

### Example 25

### In Silico Validation of TCR Specificity Groups Using HLA Tetramer Sequences

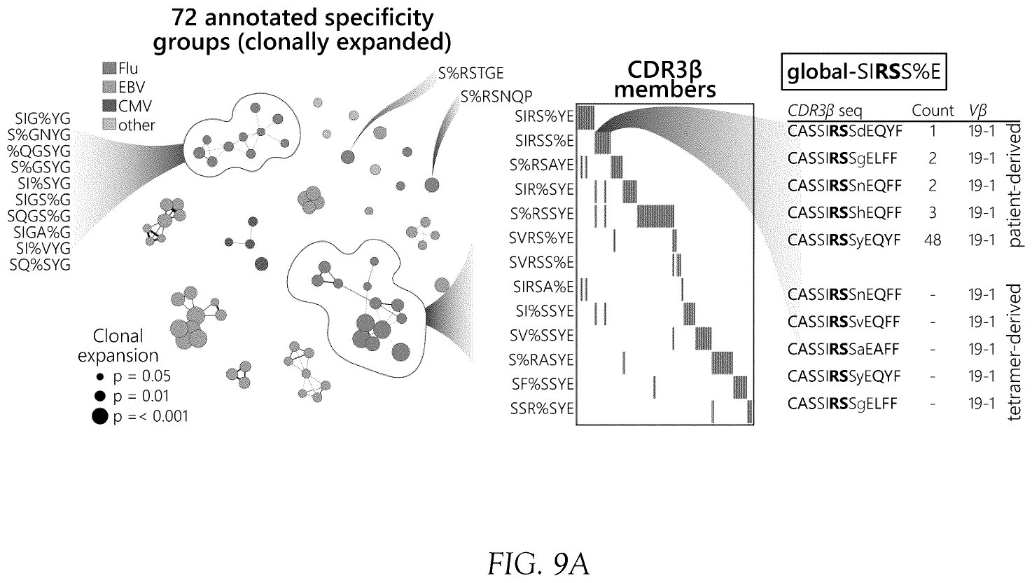

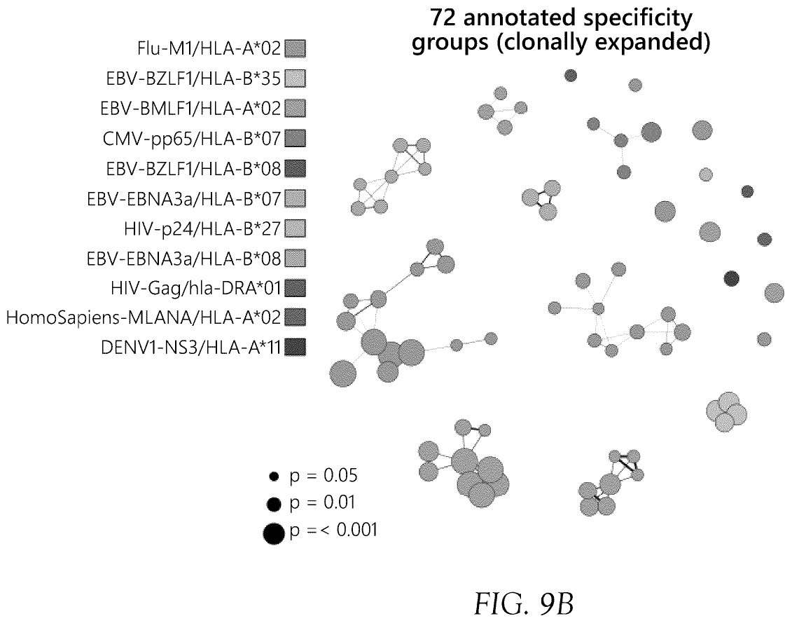

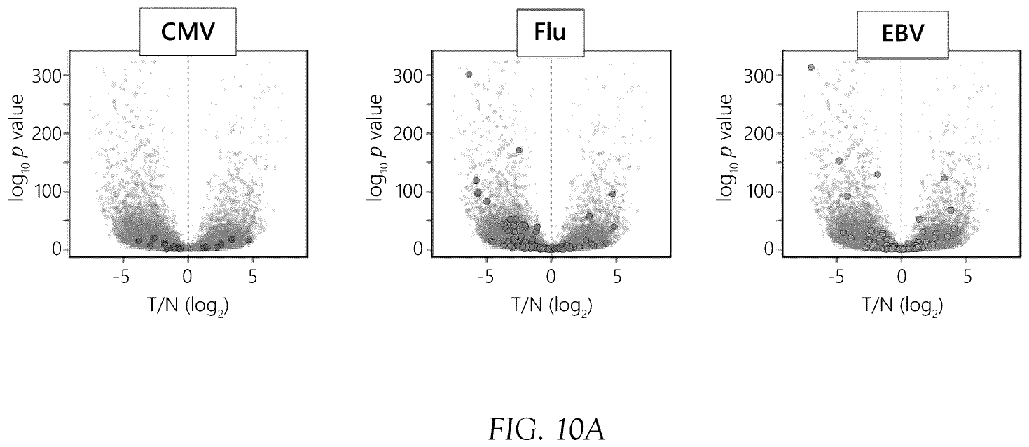

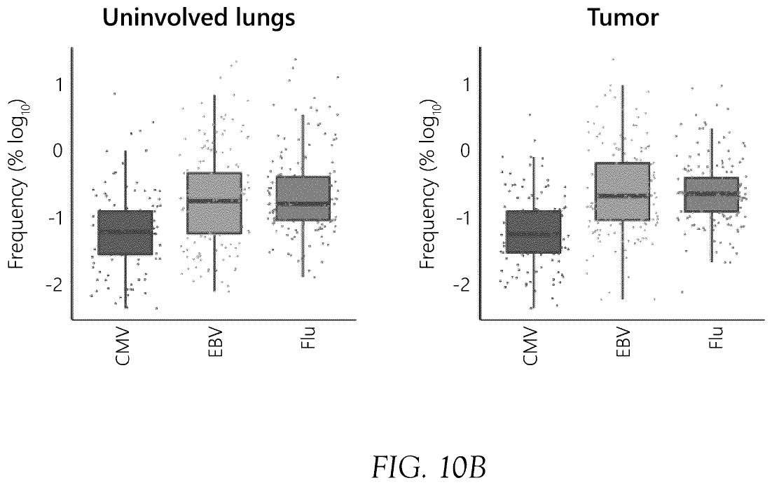

In order to validate the specificity groups established by GLIPH2, publicly available CDR3β sequences from various HLA tetramer databases were included in combination with the MD Anderson CDR3β sequences for a joint GLIPH2 analysis (Glanville, J., et al., 2017 supra; Shugay, M., et al., Nucleic Acids Res, 2018. 46(D1): p. D419-D427; and Song, I., et al., Nat Struct Mol Biol, 2017. 24(4): p. 395-406). The CDR3β sequences available from the tetramer datasets primarily cover viral specificities and have been experimentally shown to bind epitopes in the context of their respective HLAs. This allows us to annotate some of the specificity groups with sequences from the tetramer databases linked to experimentally-established antigen specificities and HLA restrictions. The joint analysis led to the annotation of 396 specificity groups (FIG. 1C). Of these specificity groups, 72 were clonally expanded and annotated with 11 different tetramers (FIG. 9). As anticipated, it was found that clonotypes with inferred specificities to Flu, Epstein-Barr virus (EBV), or CMV antigens were not preferentially localized in the tumor compared to uninvolved lung (FIG. 10A). Twelve of the 27 clonally expanded FluM1-annotated specificity groups carry either the “RS” or “GxY” motifs that are known to be critical for their engagement with FluM1 tetramer HLA-A*02/GILGFVFTL (SEQ ID NO: 23), further supporting the validity of the annotations (FIG. 9A) Song, I., et al., 2017 supra). In addition, network analysis organized these tetramer-annotated specificity groups sharing some identical CDR3β sequence members into communities (FIGS. 1C and 9). Specificity groups belonging to a given community were consistently annotated with identical HLA tetramers (FIGS. 1C and 9), indicating that some antigen specificity groups, albeit sharing distinct sequence motifs, are likely related to the same specificity and HLA restriction.

### Example 26

### Inference of HLA Restriction

This Example describes the results from experiments performed to validate the HLA alleles predicted by GLIPH v2. In these experiments, the degree to which enrichment of an HLA allele within a specificity group reflected the HLA context annotated by the tetramer was examined. The enrichment of HLA alleles that belonged to a given supertype across all of the 72 specificity groups annotated with CDR3β sequences from the tetramer dataset (Sidney, J., et al., BMC Immunol, 2008. 9: p. 1; and Harjanto, S. et al., PLoS One, 2014. 9(1): p. e86655) was quantified. The analysis was focused on HLA alleles that belonged to the HLA-A*02 and HLA-B*08 supertypes since these tetramer-defined HLA contexts were the most abundant in the MD Anderson dataset (FIG. 9B). It was hypothesized that if a given specificity group were annotated by an HLA/peptide tetramer, there should be a higher probability of observing enrichment of HLA allele(s) belonging to the same supertype by GLIPH2. Indeed, 36.0% of all HLA-A*02 tetramer-annotated specificity groups were enriched with HLA-A*02 supertype alleles, while none of the groups annotated with non-A*02 tetramers were enriched (FIG. 1D). Similarly, while 62.5% of HLA-B*08 tetramer-annotated specificity groups were enriched with HLA-B*08 supertype alleles, only 3.13% of the non-B*08 tetramer-annotated groups were enriched with HLA-B*08 supertype alleles (FIG. 1D). Therefore, the enrichment of a given HLA allele within a specificity group accurately reflected the HLA context of the cognate antigen. Previous work had also validated the inferred HLA restricting element by the more laborious and lower throughput method of expressing TCR heterodimers in reporter T cells and identifying their peptide-MHC specificity (Glanville, J., et al., 2017 supra and Huang, H., et al., 2020 supra).

### Example 27

### Establishing a Comprehensive Set of TCR Specificity Groups Derived from NSCLC Patients

One of the major advantages of establishing TCR specificity groups with GLIPH2 is that it facilitates TCR repertoire analysis across individuals. This has been previously limited by the immense diversity of TCR sequences, making it extremely challenging to derive meaningful information from deep sequencing of TCRs beyond the descriptive level (Robins, H. S., et al., Sci Transl Med, 2010. 2(47): p. 47ra64 and Arstila, T. P., et al., Science, 1999. 286(5441): p. 958-61). Given the sequencing depth of the MD Anderson lung cancer dataset, only an average ˜0.4% of the repertoire was shared between any two patients, consistent with previous reports (FIG. 1E). However, the likelihood of measuring such shared specificities increased to 1.9% when considering the 4,300 specificity groups derived from NSCLC patients or 5.3% if all specificity groups regardless of clonal expansion were used (FIG. 1E). To further illustrate that GLIPH2 specificity inferences can estimate tetramer-derived measurements, it was further shown that the estimated frequencies of clonotypes with inferred pathogen specificities were within the previously reported ranges measured by HLA tetramer staining (FIG. 10B) (Simoni, Y., et al., Nature, 2018. 557(7706): p. 575-579 and Rosato, P. C., et al., Nat Commun, 2019. 10(1): p. 567).

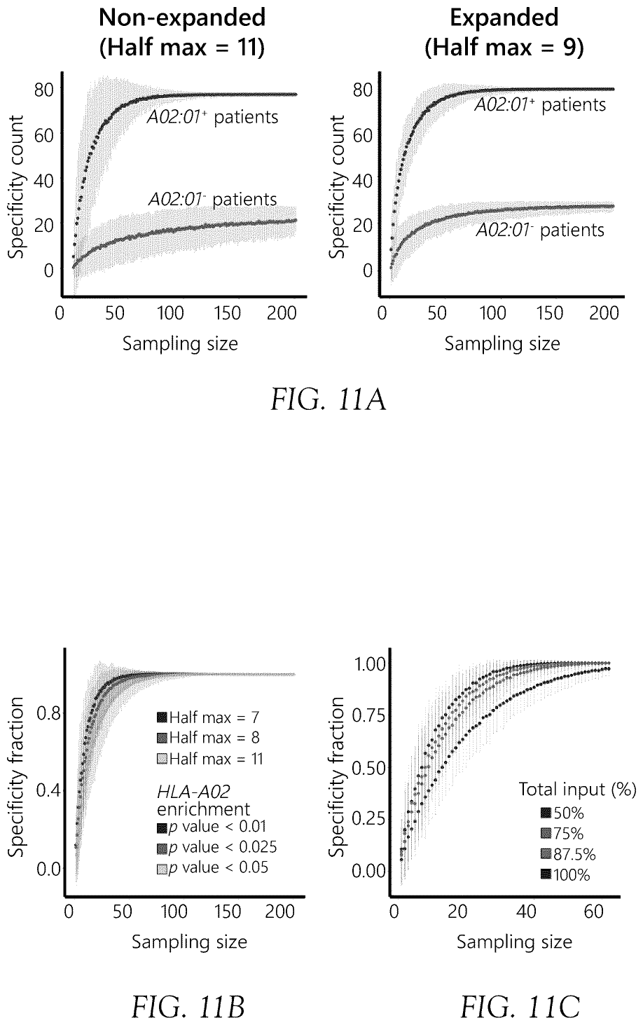

Next, it was reasoned that if a finite number of shared antigens exist in a particular disease context, then the number of specificity groups should also reach saturation given enough patients. Consistent with this reasoning, it was observed that the specificity groups reached saturation within a given HLA allele context (FIG. 1F). The minimum numbers of patients needed to cover half of the specificity groups enriched with a given HLA allele was then estimated. By bootstrapping from patients who carry at least one copy of the most prevalent HLA-A*02:01 allele, it was found that repertoires from at least 9 patients were needed to account for half of the clonally expanded specificity groups (n=77) enriched for HLA-A*02:01 (FIG. 1F). In contrast, concurrent bootstrapping from A*02:01−patients was able to account for far fewer A*02:01-enriched specificity groups (FIG. 1F). In addition, the number of patients needed to reach half the maximum specificity groups was dependent of the level of clonal expansion, the absolute numbers of specificity groups, as well as the sequencing depth of the repertoires (FIG. 11). Furthermore, a similar low coverage was observed in an independent, healthy A02+ cohort, emphasizing that these specificity groups were much more prevalent in NSCLC patients with the A*02:01 allele (FIG. 1G). Thus, a complete set of shared TCR specificity groups in lung cancer can be established with finite patient numbers. Furthermore, these results indicate that the inference of T cell specificity is strengthened by including an additional cutoff for the enrichment of specific HLA alleles.

### Example 28

### Experimental Validation of GLIPH2-Inferred Specificities

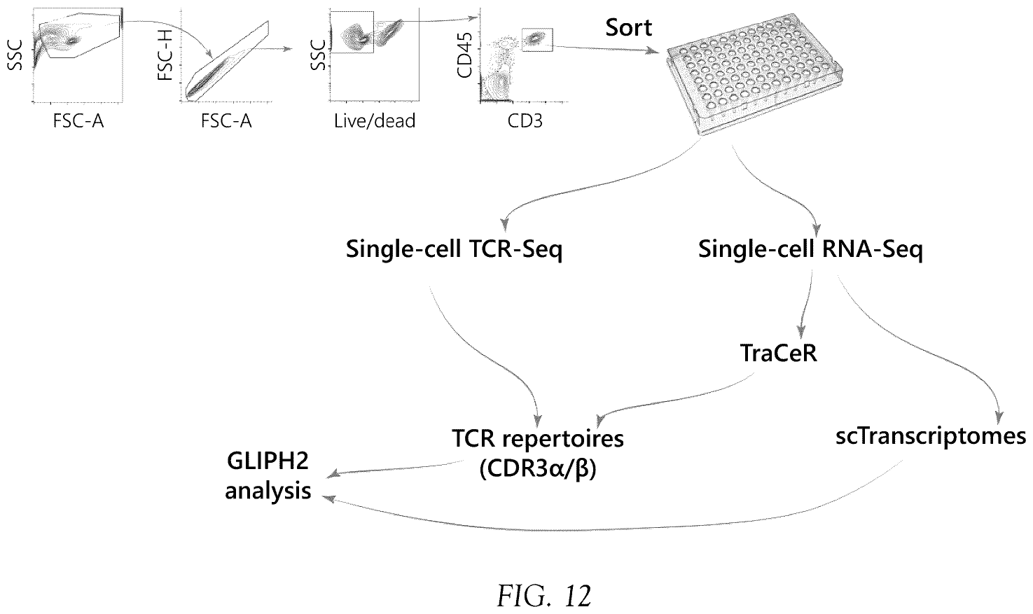

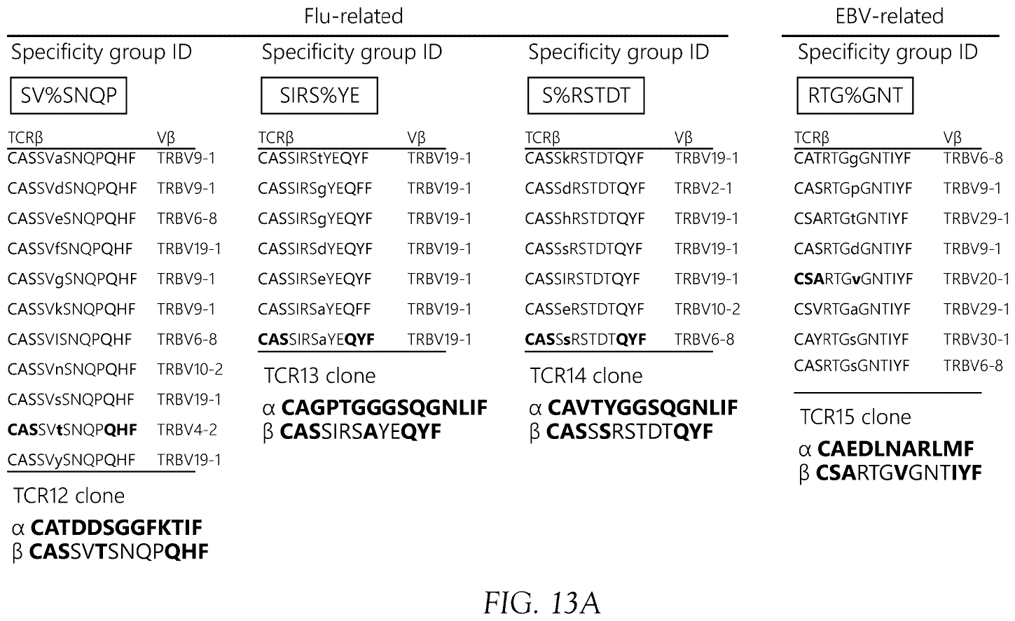

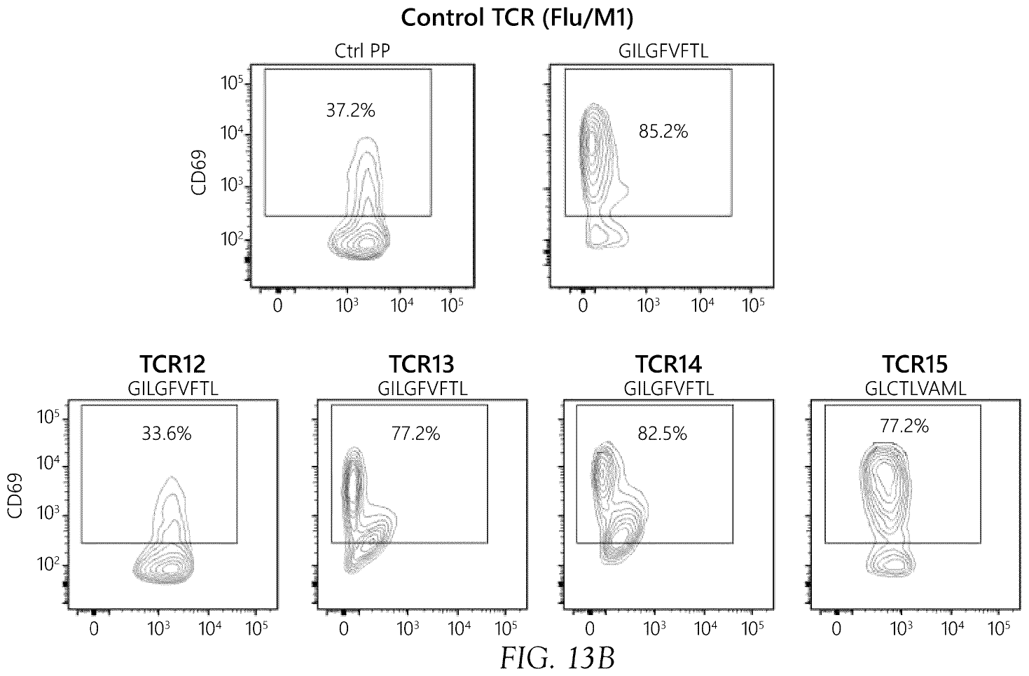

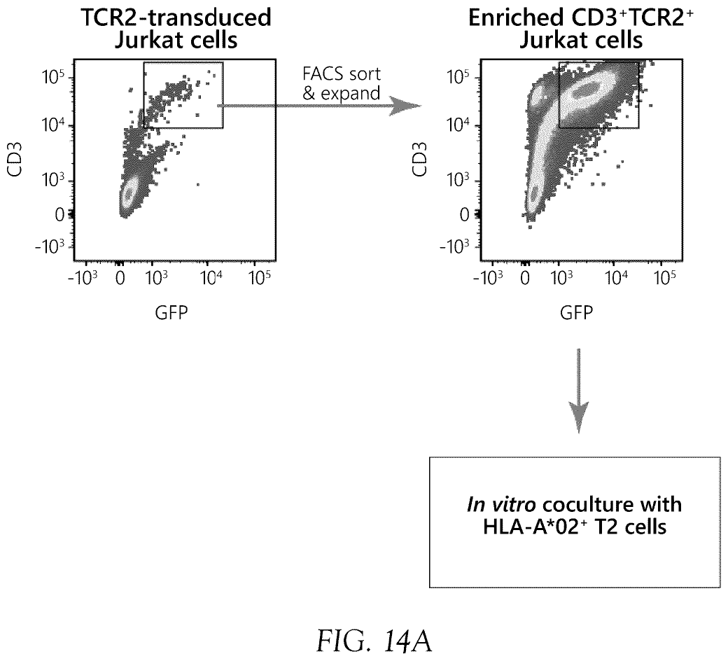

In order to experimentally validate peptide-MHC specificities, sequences from TCRα/β pairs are required from single T cells. Therefore, single-cell TCR sequencing (TCR-seq) from 15 early-stage NSCLC patients treated at Stanford was performed. Tumor-infiltrating T cells were prepared from surgically resected specimens and sorted by FACS before sequencing (FIG. 12). A total of 4,704 paired CDR3α and CDR3β sequences were sequenced and combined the CDR3β sequences with the dataset from MD Anderson. Three specificity groups inferred to recognize Flu [TCR12 (“SV % SNQP” CDR3β motif; SEQ ID NO: 50), TCR13 (“SIRS % YE” CDR3β motif; SEQ ID NO: 51), and TCR14 (“S % RSTDT” CDR3β motif; SEQ ID NO: 52)] and one specificity group inferred to recognize EBV (TCR15, “RTG % GNT” CDR3β motif; SEQ ID NO: 49) were chosen for validation. Jurkat cell clones engineered to express the four TCR candidates were used and co-cultured with T2 cells as antigen-presenting cells in the presence of their respective peptides (FIG. 13). It was found that three of them—TCR13, TCR14 and TCR15—responded to their predicted antigens in the context of HLA-A*02, which showed the robust T cell specificity inferences using GLIPH2 (FIG. 13).

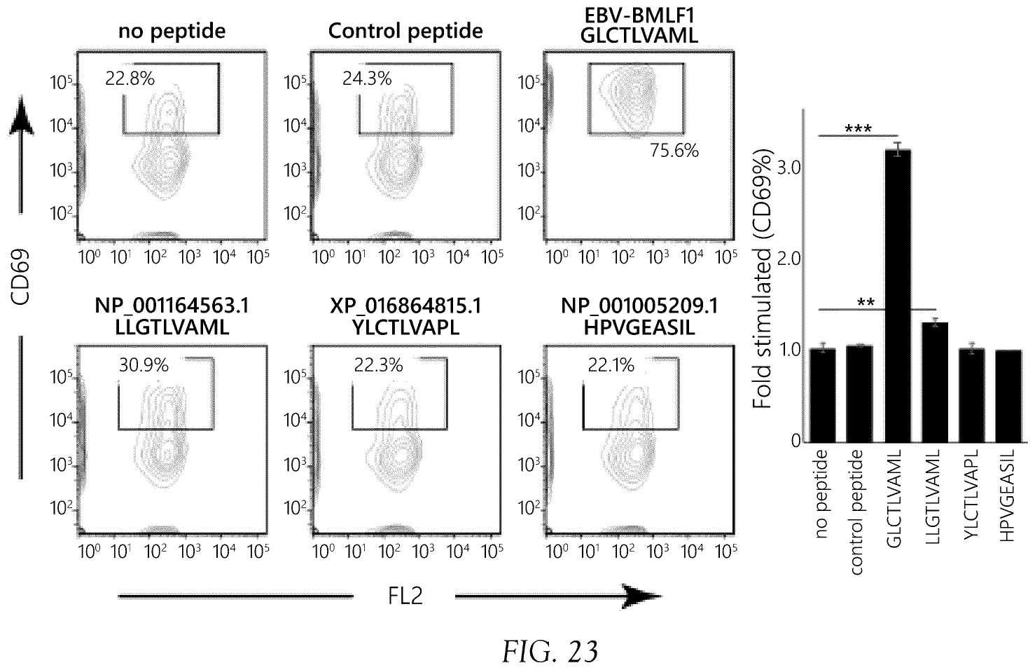

FIG. 23 shows representative FACS plots shown the stimulation of the Jukat-TCR cells with 9 mers from the EBV BMLF1 locus “GLCTLVAML” (SEQ ID NO: 44), uniprot NP 001164563.1 (CLDN2 locus, LLGTLVAML; SEQ ID NO: 45), XP 016864815.1 (SERINC5 locus, YLCTLVAPL; SEQ ID NO: 46), and NP 001005209.1 (TMEM198 locus, HPVGEASIL; SEQ ID NO: 47). Right panel: results of Jurkat-TCR15 cell stimulation in triplicate. Control peptide: flu M1 “GILGFVFTL” (SEQ ID NO: 23). *** p<0.001; **, p<0.01 by student t test. In conclusion, the experimental results indicate that the tumor-derived clone TCR15 is likely cross-reactive to a shared tumor antigen (CLDN2) and the viral antigen from EBV. Accordingly, the CDR3β TCR15 sequences described in the present disclosure may be useful in the development of recombinant T-cell receptors that therapeutically target CLDN2-positive cancers. Multiple human cancers have been previously reported to express CLDN2 protein. In particular, high levels of CLDN2 have been previously observed in colorectal cancer, cervical cancer, liver cancer, lung cancer, gastric cancer, pancreatic cancer, renal cancer, and stomach cancer. More information in this regard can be found, for example, in the Human Protein Atlas, which is publicly available at www.proteinatlas.org/ENSGO0000165376-CLDN2/pathology.

### Example 29

### Identification of a T Cell Specificity Group Cross-Reactive to Tumor and Pathogen-Derived Antigens in Human Lung Cancer

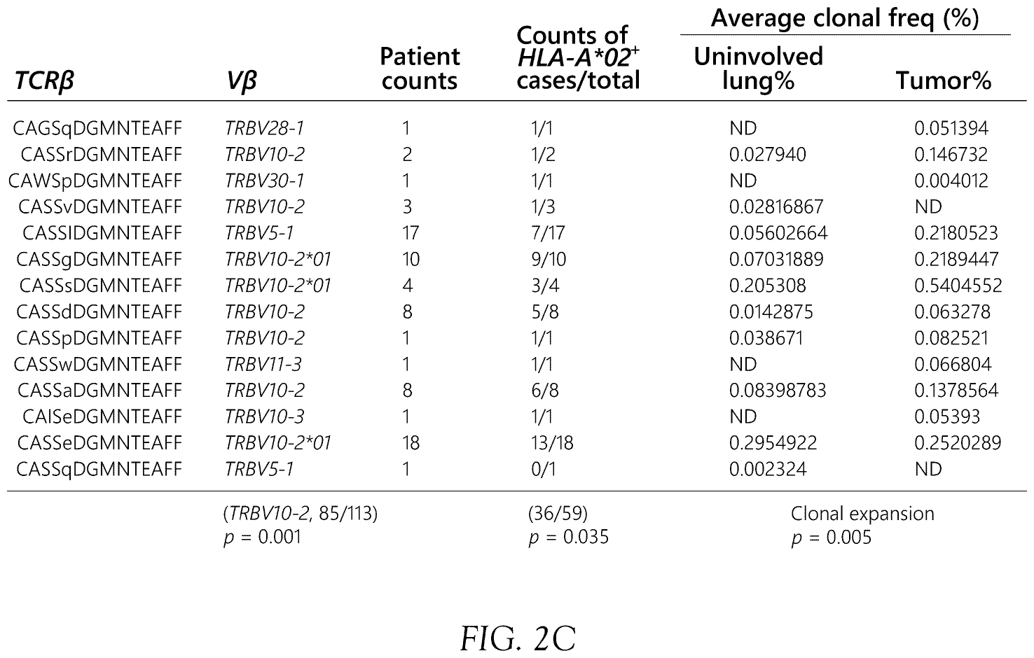

To identify novel shared tumor antigens, the repertoires from both tumors and uninvolved lungs from NSCLC patients and prioritized TCR specificity groups enriched in tumors were leveraged (FIGS. 2A and 7). Specifically, the CDR3β frequencies for the 4,300 clonally expanded specificity groups between tumors and uninvolved lungs were compared using the Poisson test (FIG. 2A). This led to the identification of 449 specificity groups that are significantly enriched with CDR3β clonotypes localized in tumor, suggesting that these clonotypes may recognize tumor antigens that are shared across multiple patients (FIG. 2A). Of the 449 tumor-enriched specificity groups, the priority was made for those that fulfilled the criteria of (1) having a paired TCRα/β clonotype from the Stanford cohort and (2) significantly enriched with HLA-A*02 alleles (FIGS. 2B and 2C). This led us to identify the specificity group with the “S % DGMNTE” CDR3β motif (SEQ ID NO: 48) where the amino acid “%” varied (FIG. 2C). Hence, the candidate TCRWp clonotype (referred to as TCR2) bearing the CDR3α sequence CAVLMDSNYQLIW (SEQ ID NO: 24) and CDR3β sequence CASSGDGMNTEAFF (SEQ ID NO: 6) was chosen for antigen identification (FIG. 3A).

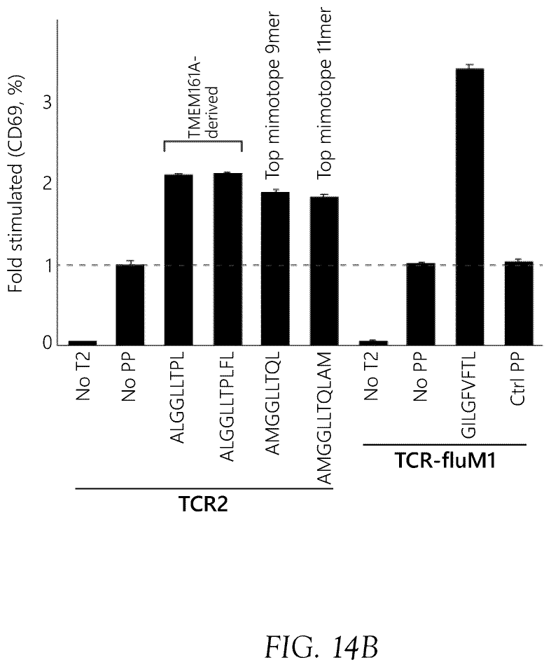

To screen for the cognate epitopes of the candidate clone TCR2, the previously reported yeast libraries displaying peptides of 4 different lengths (8-11 amino acids) were used in the context of wildtype HLA-A*02:01(Gee, M. H., et al., 2018 supra). Four rounds of affinity-based selection of yeast clones with the recombinant TCR2a/p soluble proteins led to the enrichment of peptide sequences (mimotopes) only in the 111 mer library (data not shown). An in vitro stimulation assay was performed with the top-20 enriched mimotopes and showed that the top two sequences “AMGGLLTQLAM” (SEQ ID NO: 15) and “KLGGLLTMVGV” (SEQ ID NO: 18) stimulated Jurkat cells expressing TCR2 when co-cultured with HLA-A*02+ T2 cells (FIGS. 3A and 14A). A protein database search (UniParc) led to the identification of multiple endogenous 9mers that resembled the top two mimotopes from the yeast library screen and were predicted to bind HLA-A*02:01 with anchors separated by 6 instead of 8 amino acids (FIG. 3B). Indeed, the 9mer variant of the top two enriched mimotopes stimulated the Jurkat TCR2 clone to the comparable level of the 11mer counterparts (FIG. 14B). This result suggested that the identified HLA-A*02 antigens were defacto 9mers.

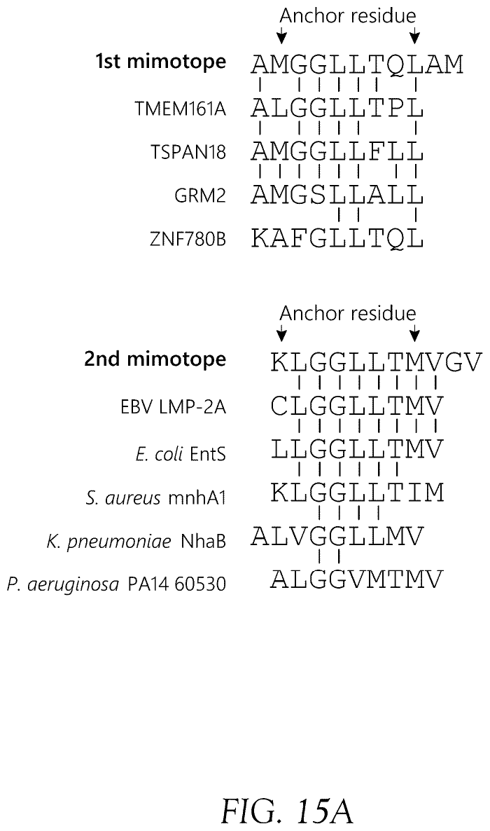

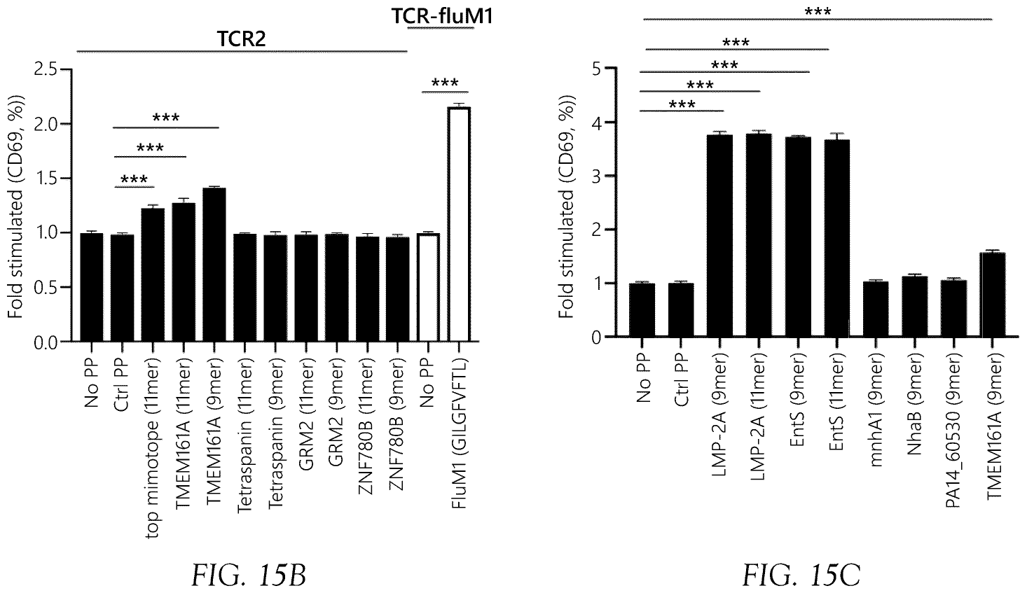

The ability of the candidate endogenous peptides resembling the top two mimetopes was functionally tested in the yeast library screen to stimulate Jurkat cells expressing TCR2 (FIGS. 3C and 15). It was found that 9mers and 11 mers from the mammalian protein TMEM161A (TMEM9mer, ALGGLLTPL, SEQ ID NO: 17 and TMEM11 mer, ALGGLLTPLFL, SEQ ID NO: 25), the latent membrane protein 2a (LMP9mer, CLGGLLTMV, SEQ ID NO: 19 and LMP1I1mer, CLGGLLTMVSA, SEQ ID NO: 20) from EBV, and the enterobactin exporter (EntS9mer, LLGGLLTMV, SEQ ID NO: 21 and EntS11 mer, LLGGLLTMV, SEQ ID NO: 22) from E. coli could all stimulate the Jurkat TCR2 clone when co-cultured with HLA-A*02+ T2 cells (FIGS. 3C and 15). Furthermore, to show that the full-length proteins TMEM161A, LMP2, and EntS could be processed, presented, and loaded on the HLA-A*02:01 and activate T cells, these full-length proteins were then overexpressed in HLA-A*02+293T cells and the responses from co-cultured primary T cells expressing TCR2 were measured. Similar to the pulsed peptides, full-length proteins from TMEM161A, LMP2, and EntS could all be processed into peptides that stimulate primary T cells expressing TCR2, with the human TMEM161A appearing to be the weakest stimulant of the three (FIG. 3D). More importantly, identification of the cognate antigens for TCR2 further demonstrated that GLIPH2 correctly predicted the HLA restriction of “S % DGMNTE” (SEQ ID NO: 48) specificity group. In summary, combining GLIPH2 and yeast antigen library screening led us to identify a T cell specificity group and the cross-reactive antigens from tumor and pathogens.

### Example 30

### TMEM161A is Overexpressed on Human Lung Cancer

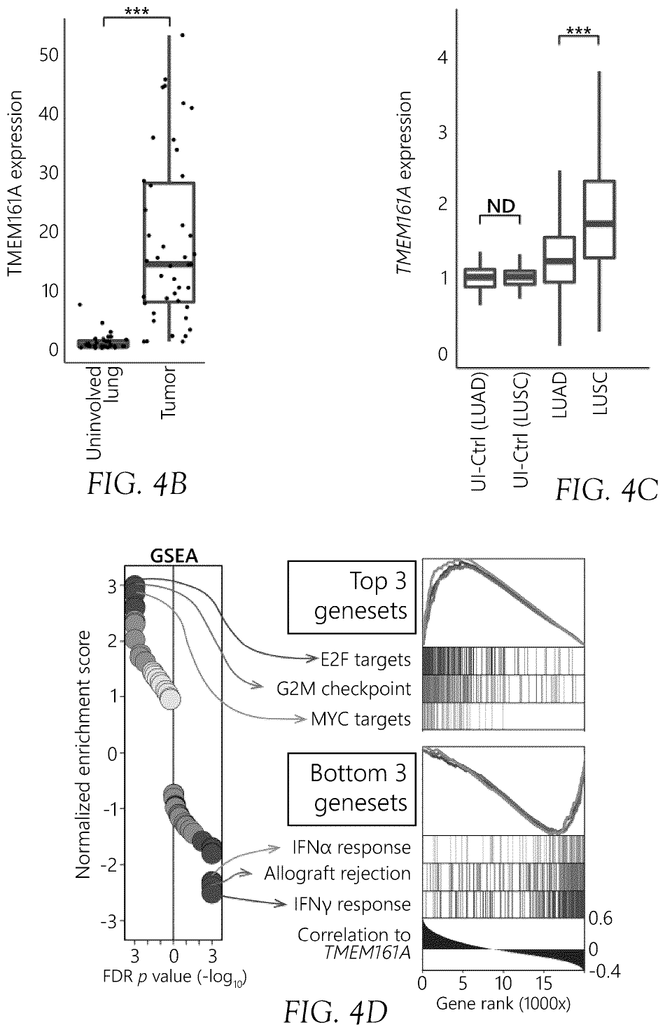

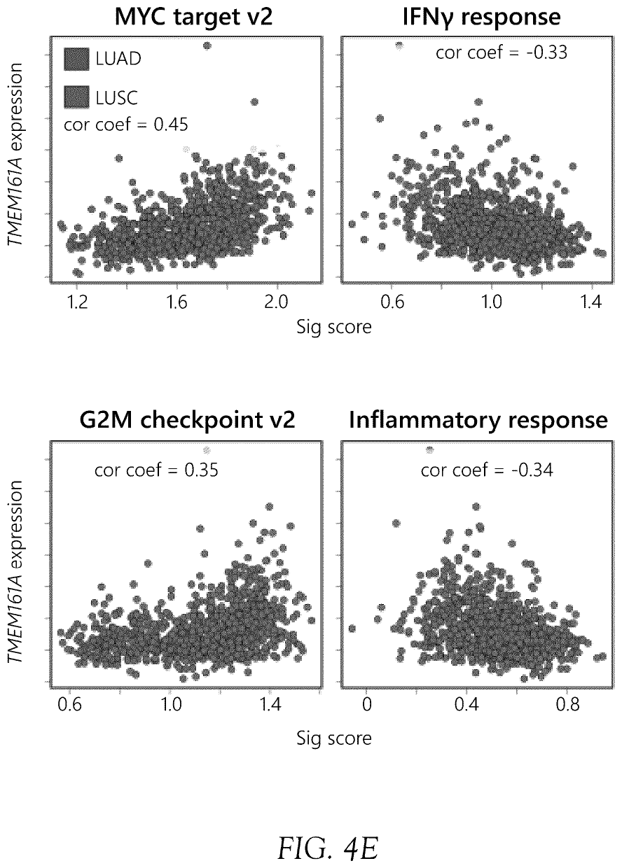

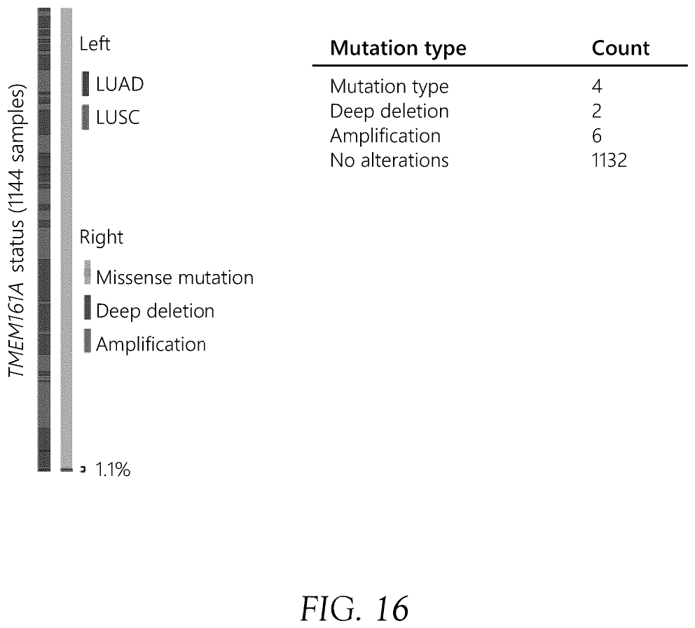

Significantly higher levels of TMEM161A protein expression in human lung cancer compared to the uninvolved lungs (FIGS. 4A and 4B) were found. In particular, TMEM161A was found broadly expressed on human NSCLC tumors (see, e.g., FIGS. 4A, 4B, and 4C; n=900 subjects). To examine TMEM161A expression in a larger lung cancer cohort, TMEM161A RNA expression was examined with the Cancer Genome Atlas (TCGA) dataset. Consistent with protein expression, higher levels of TMEM161A expression were found in tumor compared to the uninvolved lung from NSCLC patients. Moreover, the level of TMEM161A expression was higher in squamous cell carcinomas of the lung compared to adenocarcinomas (FIG. 4C). Whole-exome sequencing of specimens from the Stanford cohort did not identify any mutation within the coding region of the TMEM161A locus, supporting its role as a non-mutated tumor antigen (data not shown). Similarly, less than 1% of deleterious mutations in the TMEM161A locus were found in the pan-lung cancer TCGA dataset (n=6/1053, FIG. 16). Interestingly, gene set enrichment analysis (GSEA) showed TMEM161A expression in lung cancer was associated with signatures related to cell proliferation programs and the proto-oncogene MYC targets (FIGS. 4D and 4E). In contrast, TMEM161A expression appeared to correlate negatively with gene sets related to inflammatory responses (FIGS. 4D and 4E). Altogether, the results from the GSEA analysis show that TMEM161A expression in tumors was inversely correlated with expression of genes associated with an anti-tumor immune response.

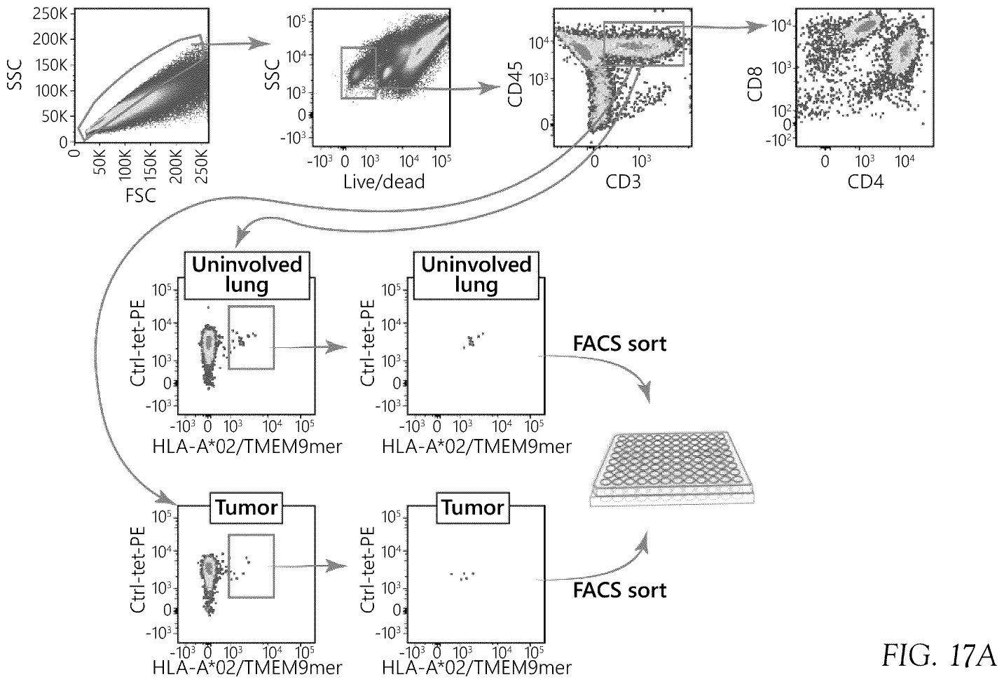

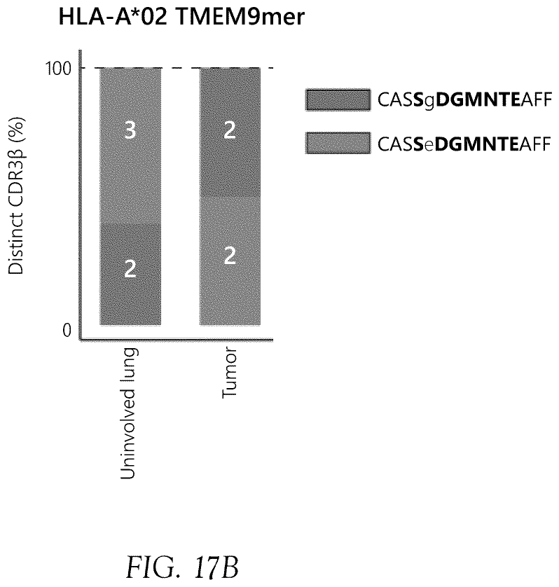

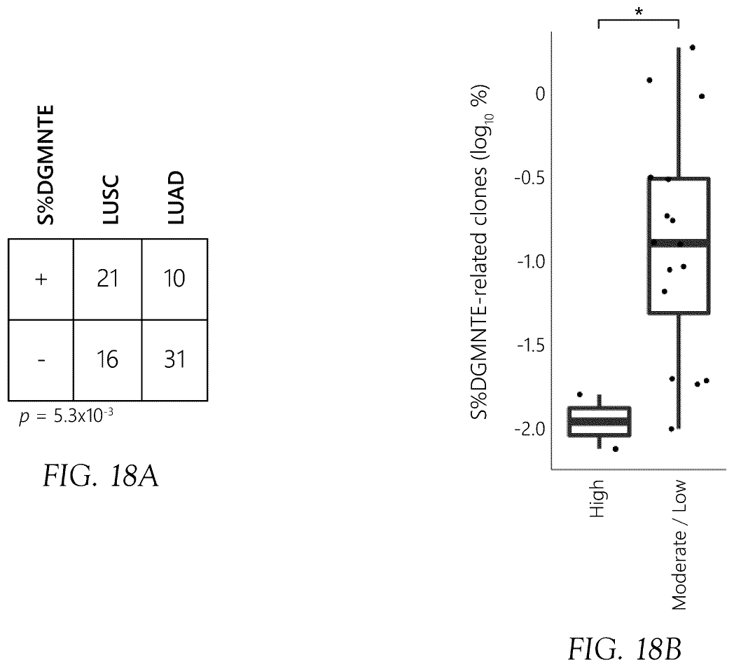

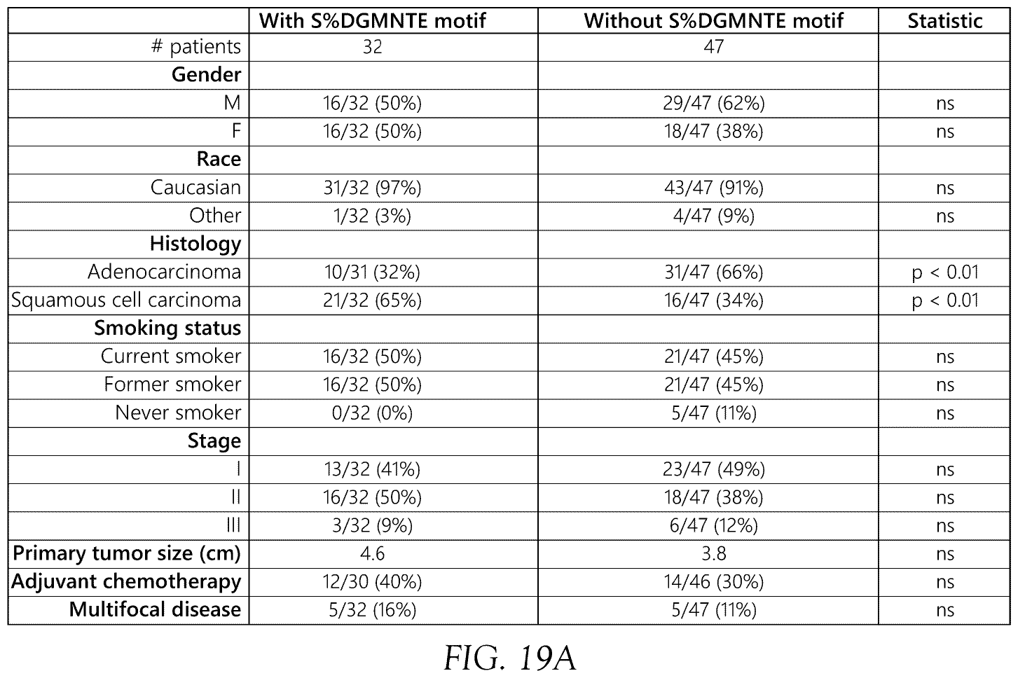

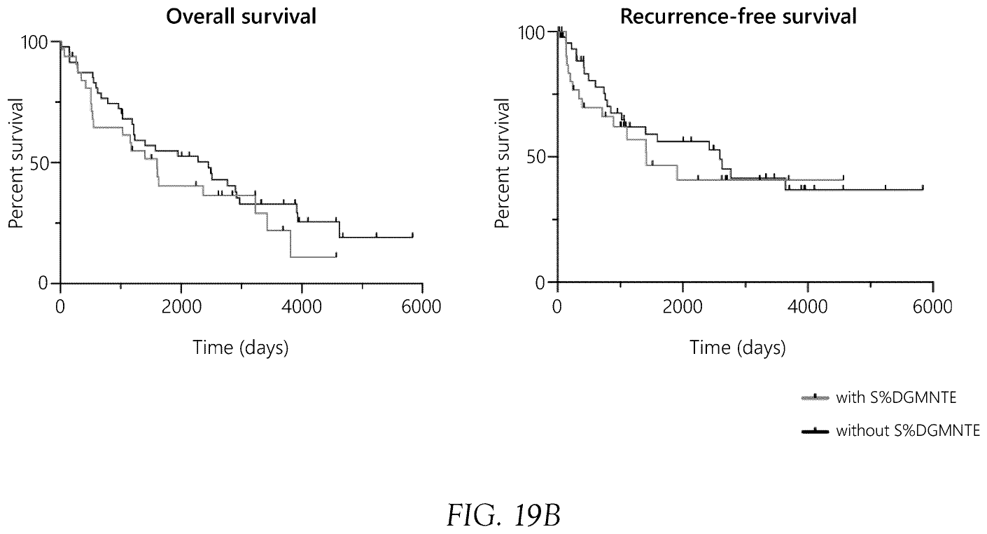

In addition to examining TMEM161A expression in tumors, additional experiments were performed to further characterize TMEM161A-specific CD8+ T cells. HLA-A*02 tetramers loaded with TMEM9mer were used to sort T cells from single-cell suspensions of tumor cells from the HLA-A*02+ patient where these T cells were first identified (patient A6). Single-cell TCR-seq of these TMEM9mer/A02 tetramer+ T cells from tumor and uninvolved lung confirmed that they carried the “S % DGMNTE” motif (SEQ ID NO: 48), consistent with their recognition of TMEM161A in vivo (FIG. 17). The question of how tumor characteristics impact the recruitment of T cells with the “S % DGMNTE” CDR3β motif (SEQ ID NO: 48) among patients who were HLA-A02+ was next examined. It was observed that T cells with the “S % DGMNTE” CDR3β motif (SEQ ID NO: 48) were more frequently observed in squamous cell carcinomas compared to adenocarcinomas, consistent with the expression pattern of TMEM161A in these tumors (FIGS. 18A and 4C). It was also observed that these T cells were more abundant in tumors with mutation count <500 compared to tumors with mutation count >500 (n=34), suggesting that the presence of tumor-infiltrating T cells recognizing over-expressed shared antigens may be a more common feature of tumors with low to moderate mutation burdens (FIG. 18B). The presence or absence of T cells with the “S % DGMNTE” CDR3β motif (SEQ ID NO: 48) in tumors did not impact the clinical outcome of patients (FIG. 19). However, the association T cells with the “S % DGMNTE” CDR3β motif (SEQ ID NO: 48) with genetic attributes of the tumors further supports their recognition of the tumor antigen TMEM161A and not pathogen-derived antigens in vivo. Consistently, previous reports on TCGA dataset showed that EBV was rarely detected in lung cancer. In summary, the experimental data described herein has shown that TMEM161A is broadly and highly expressed in NSCLC and that T cells recognizing this antigen are found in 30/78 (38%) of HLA-A*02+ patients.

### Example 31

### Cross-Reactive CD8+ T Cells Recognizing Tumor and Pathogen Antigens are Detected in Healthy Donors

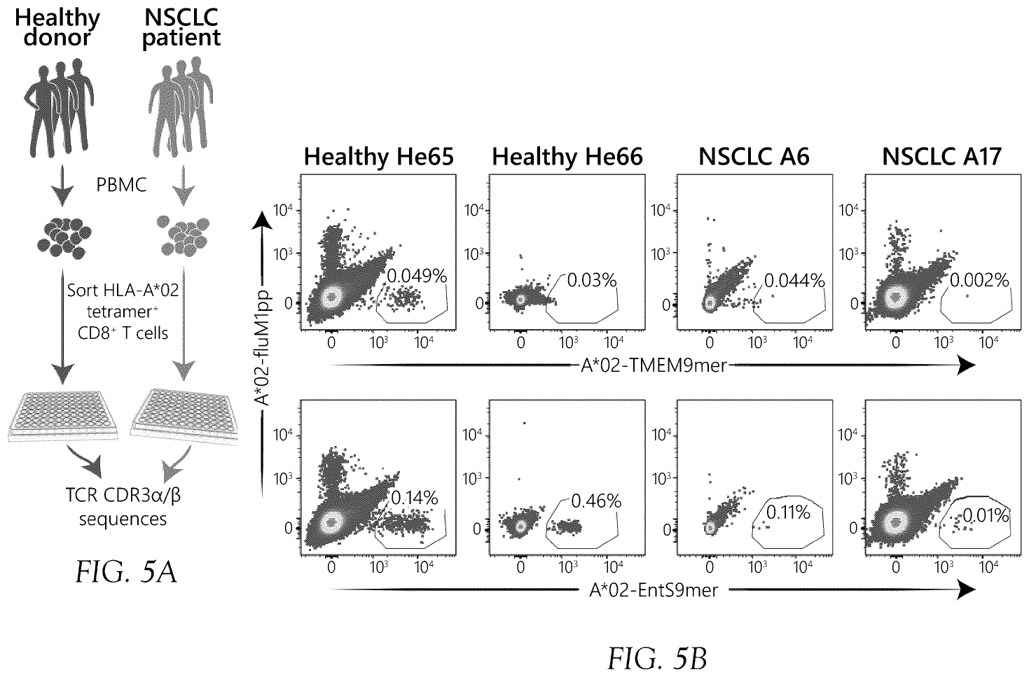

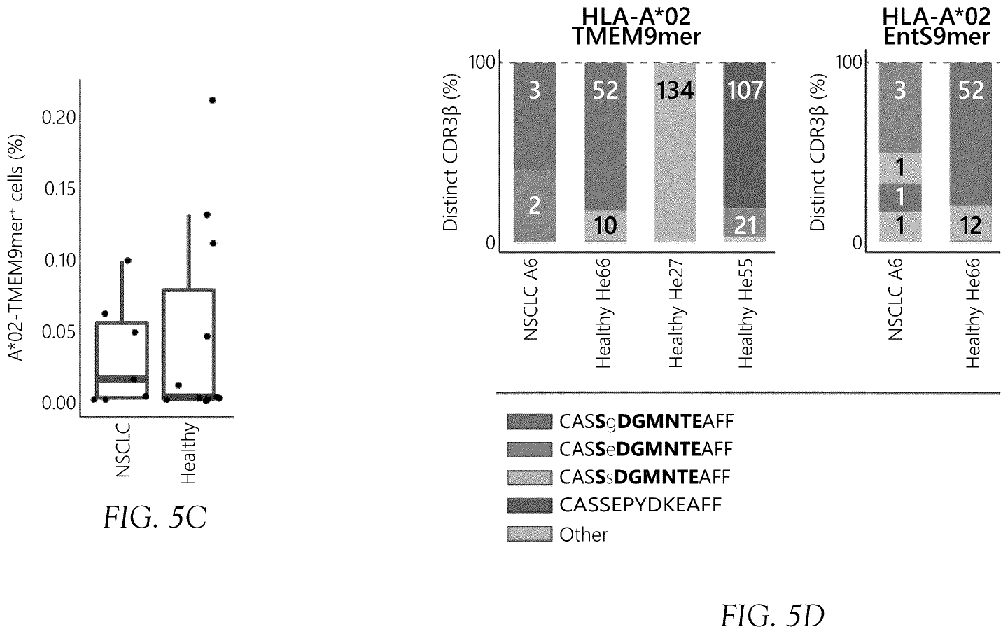

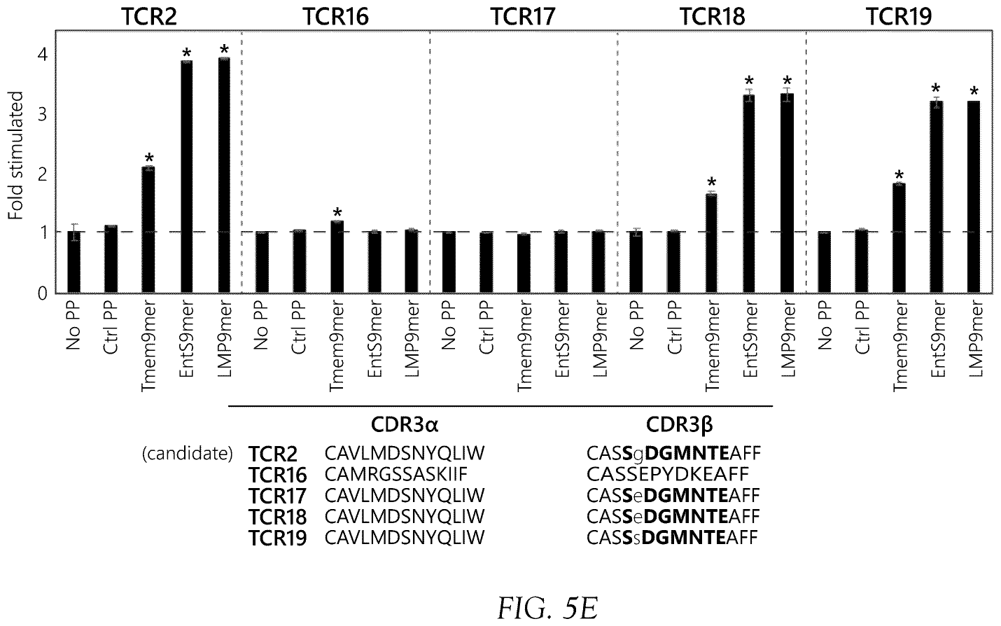

To characterize all the cross-reactive TMEM161A-specific and pathogen-specific clonotypes, HLA-A*02 tetramers loaded with either the TMEM9mer or the EntS9mer were used to sort CD8+ T cells from the peripheral blood of HLA-A*02+ healthy donors and NSCLC patients by FACS (FIG. 5A). No difference in the frequency of HLA-A*02/TMEM9mer+CD8 T cells in healthy donors and lung cancer patients (FIGS. 5B and 5C) was observed, suggesting that the frequencies of these T cells were likely being maintained due to the recognition of pathogen-derived antigens. In addition, the frequencies of these specific T cells, as directly measured by tetramers or as estimated by GLIPH2 were approximately one in every 103-105 T cells [tetramer-measured: 0.0032-0.0980%; GLIPH2-inferred: 0-0.2643%], which is elevated above the typical baseline range (one in every 105-10⁶ T cells).

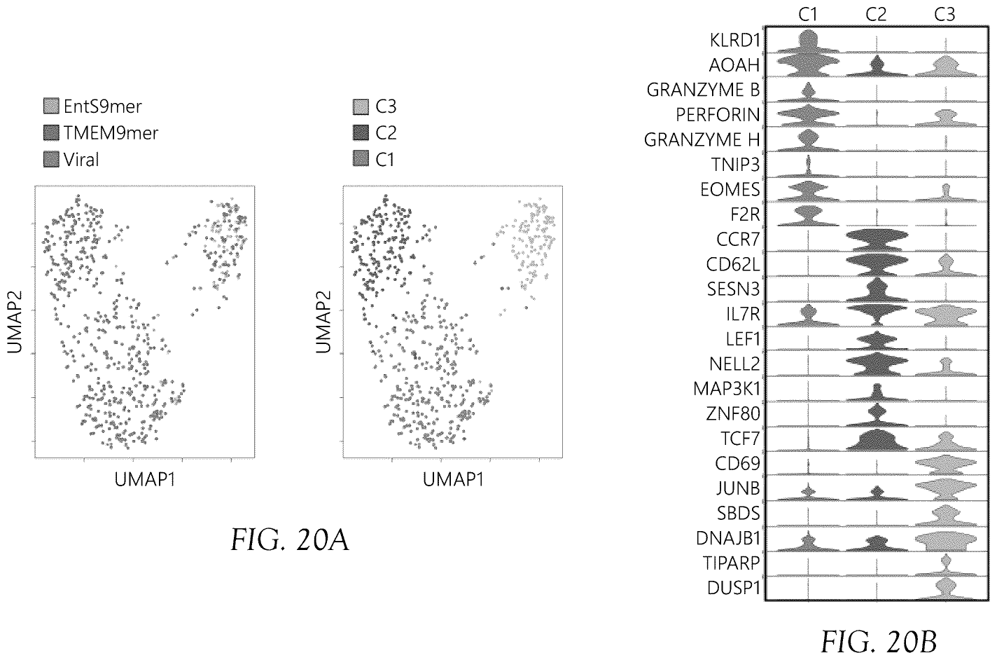

Regardless of whether TMEM9mer/A*02 tetramers or EntS9mer/A*02 tetramers were used to sort peripheral blood T cells, the CDR3β sequences of the sorted cells were consistently enriched with the “S % DGMNTE” sequence motif (SEQ ID NO: 48) (FIG. 5D). In fact, a variety of CDR3β sequences sharing the “S % DGMNTE” motif (SEQ ID NO: 48) where % could be a glycine, glutamate, or serine was found, confirming the diversity seen in the GLIPH2 specificity group carrying this motif (FIG. 5D). Furthermore, scRNA-Seq data suggested that the sorted cells mostly had effector T cell states, indicating that they have encountered their cognate antigens, even in healthy individuals (FIG. 20). To functionally validate CDR3α/β sequences from the tetramer-sorted clones, stable Jurkat cells expressing the TCRα/β chains identified with the tetramers were generated. Their reactivity to both TMEM9mer and pathogen-derived 9mers in the context of HLA-A*02:01 was then quantified. It was found that the Jurkat cell clones with the “S % DGMNTE” CDR3β motif (SEQ ID NO: 48) could cross-react to TMEM9mer, EntS9mer, and LMP9mer peptides only when paired with the permissive TCR2a chain (CDR3a: CAVLMDSNYQLIW, FIG. 5E). Furthermore, an A*02:01/TMEM9mer-specific clonotype identified with the tetramer sort that did not carry the “S % DGMNTE” motif (SEQ ID NO: 48) within the CDR3β sequence was also tested (FIG. 5E). This clonotype appeared to be slightly reactive to TMEM9mer stimulation but did not cross-react to other peptides (FIG. 5E). In summary, CD8+ T cells with the “S % DGMNTE” CDR3β motif (SEQ ID NO: 48) could cross-react to tumor antigen TMEM161A and pathogen-derived antigens EntS and LMP2 when paired with the permissive a chain.

### Example 32

### Phenotypic Characterization of the TMEM161A-Specific CD8+ T Cells

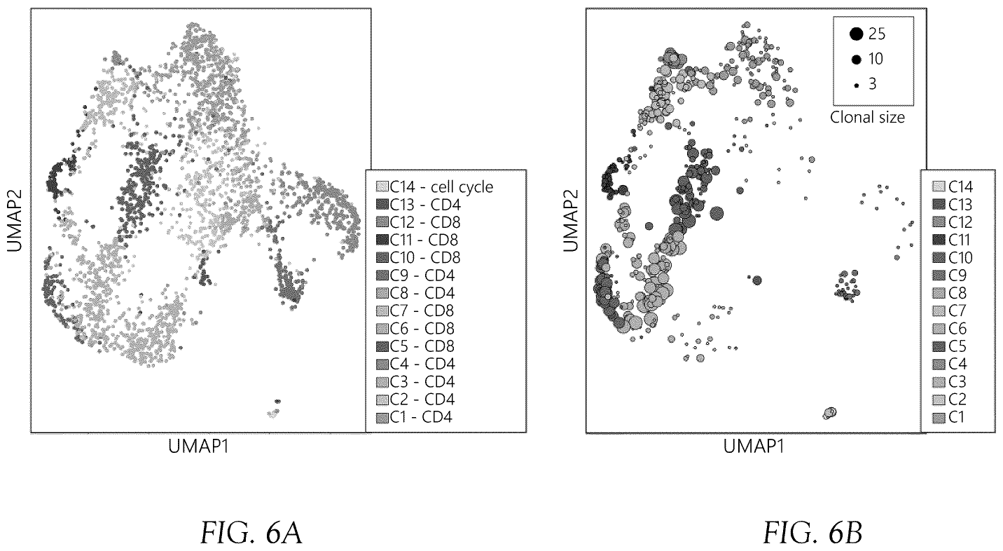

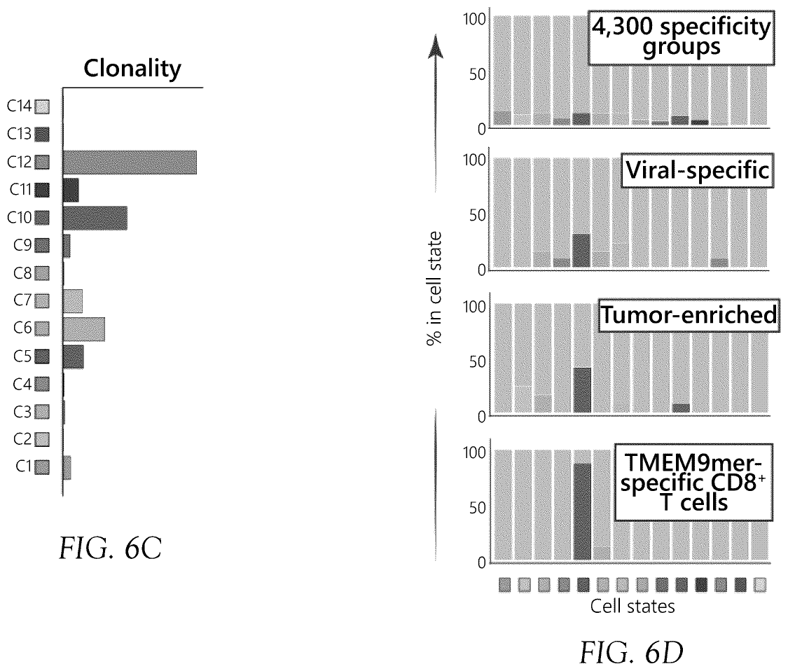

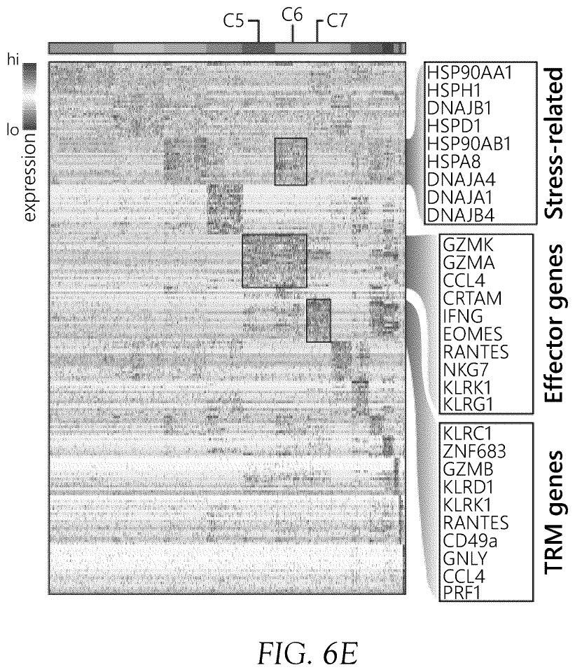

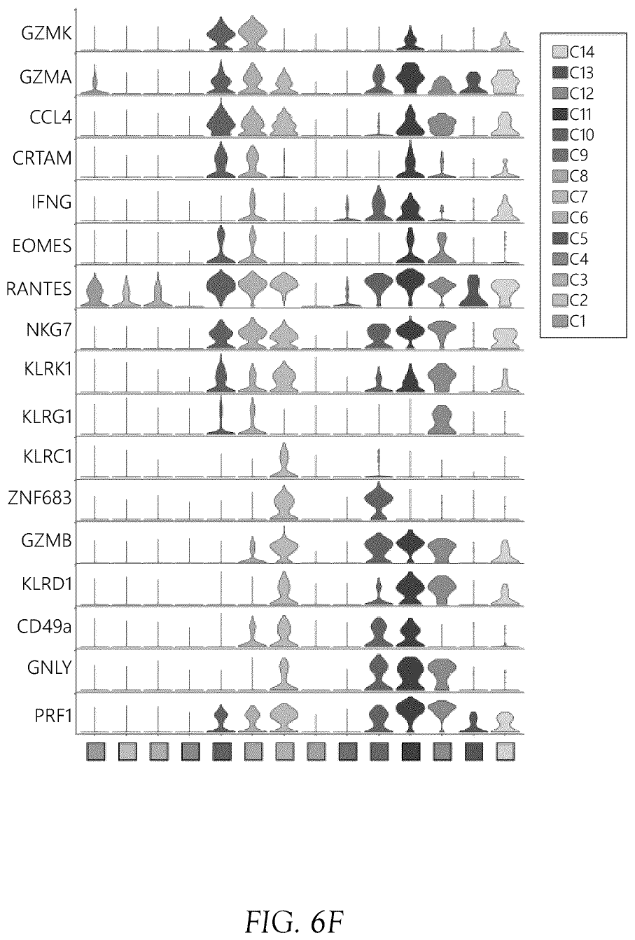

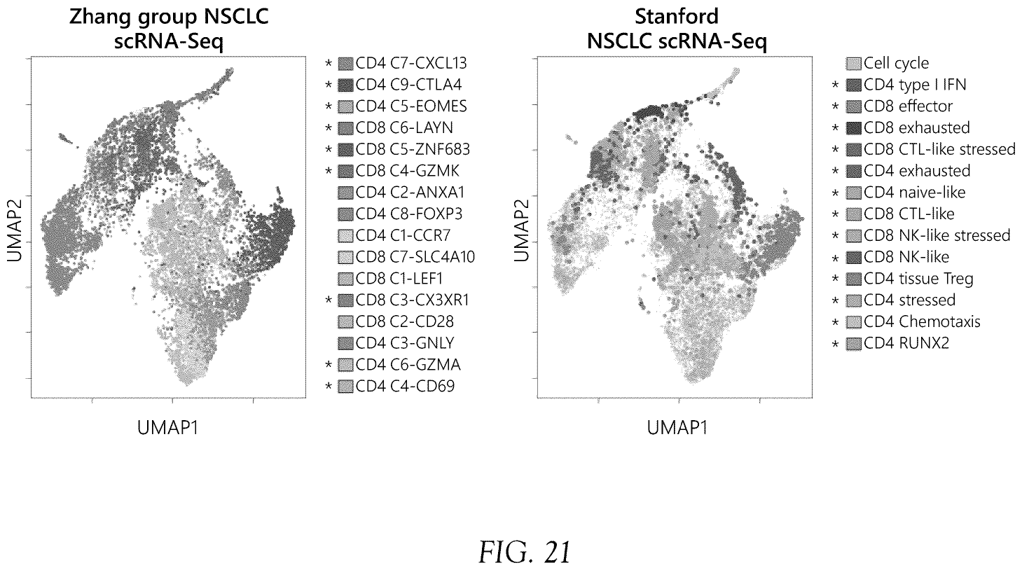

In order to assess the cell states of tumor-infiltrating T cells, the full transcriptomic profiles of 2,950 sorted T cells from 10 treatment-naïve NSCLC patients with single-cell RNA-sequencing were generated using the SMART-Seq method (scRNA-Seq). These data were linked to the TCR repertoire data (paired CDR3α/β) of the sorted T cell (FIG. 12). Fourteen major cell states were identified. Of which, 13 could be mapped to those previously described in a different NSCLC patient cohort (FIGS. 6A and 21). Among the most clonally expanded cell state clusters are c5, c6, c12 (CD8+ T cells with effector phenotypes), c7, and c10 (CD8+ T cells with resident memory phenotype) (FIGS. 6B and 6C). In order to understand the relationship between cell states and specificities against shared antigens, specificity groups using the combined Stanford and MD Anderson cohorts and examined their scRNA-Seq profiles were established. It was found that 2.9% of the T cells (n=86/2950) from the Stanford cohort belonged to the clonally expanded specificity groups (n=4,300, FIG. 6D). In addition, 12 of these T cells were members of the 449 tumor-enriched specificity groups whereas 13 of these T cells were inferred to be virus-specific (FIG. 6D). Interestingly, while T cells belonging to all the 4,300 clonally expanded specificity groups did not exhibit a bias in their cell states, T cells belonging to tumor-enriched specificity groups were biased toward effector phenotypes (c5, c6) and differentially expressing EOMES, KLRG1, and other genes expressed in activated NK cells (FIGS. 6D, 6E, and 6F). Consistently, HLA-A*02/TMEM9mer tetramer-sorted CD8+ T cells from tumor also preferentially exhibited effector T cell phenotypes in c5 and c6 (FIGS. 6D, 6E, and 6F). T cells inferred to be viral-specific exhibited cell states that included both effector phenotypes (c5, c6, c12) and tissue resident-memory phenotypes (c7). In conclusion, it was found that TMEM161A-specific CD8+ T cells reveal effector T cell states in tumors.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. Kawakami, Y., et al., Cloning of the gene coding for a shared human melanoma antigen recognized by autologous T cells infiltrating into tumor. Proc Natl Acad Sci USA, 1994. 91(9): p. 3515-9.
2. Coulie, P. G., et al., A new gene coding for a differentiation antigen recognized by autologous cytolytic T lymphocytes on HLA-A2 melanomas. J Exp Med, 1994. 180(1): p. 35-42.
3. van der Bruggen, P., et al., A gene encoding an antigen recognized by cytolytic T lymphocytes on a human melanoma. Science, 1991. 254(5038): p. 1643-7.
4. Coulie, P. G., et al., A mutated intron sequence codes for an antigenic peptide recognized by cytolytic T lymphocytes on a human melanoma. Proc Natl Acad Sci USA, 1995. 92(17): p. 7976-80.
5. Wolfel, T., et al., A p16INK4a-insensitive CDK4 mutant targeted by cytolytic T lymphocytes in a human melanoma. Science, 1995. 269(5228): p. 1281-4.
6. Murray, R. J., et al., Identification of target antigens for the human cytotoxic T cell response to Epstein-Barr virus (EBV): implications for the immune control of EBV-positive malignancies. J Exp Med, 1992. 176(1): p. 157-68.
7. Koziel, M. J., et al., HLA class I-restricted cytotoxic T lymphocytes specific for hepatitis C virus. Identification of multiple epitopes and characterization of patterns of cytokine release. J Clin Invest, 1995. 96(5): p. 2311-21.
8. Rehermann, B., et al., The cytotoxic T lymphocyte response to multiple hepatitis B virus polymerase epitopes during and after acute viral hepatitis. J Exp Med, 1995. 181(3): p. 1047-58.
9. Tran, E., et al., Immunogenicity of somatic mutations in human gastrointestinal cancers. Science, 2015. 350(6266): p. 1387-90.
10. Gros, A., et al., Prospective identification of neoantigen-specific lymphocytes in the peripheral blood of melanoma patients. Nat Med, 2016. 22(4): p. 433-8.
11. Zacharakis, N., et al., Immune recognition of somatic mutations leading to complete durable regression in metastatic breast cancer. Nat Med, 2018. 24(6): p. 724-730.
12. Schumacher, T. N., W. Scheper, and P. Kvistborg, Cancer Neoantigens. Annu Rev Immunol, 2019. 37: p. 173-200.
13. Rosenberg, S. A. and M. E. Dudley, Adoptive cell therapy for the treatment of patients with metastatic melanoma. Curr Opin Immunol, 2009. 21(2): p. 233-40.
14. Hinrichs, C. S. and S. A. Rosenberg, Exploiting the curative potential of adoptive T-cell therapy for cancer. Immunol Rev, 2014. 257(1): p. 56-71.
15. de Vos van Steenwijk, P. J., et al., An unexpectedly large polyclonal repertoire of HPV-specific T cells is poised for action in patients with cervical cancer. Cancer Res, 2010. 70(7): p. 2707-17.
16. Piersma, S. J., et al., Human papilloma virus specific T cells infiltrating cervical cancer and draining lymph nodes show remarkably frequent use of HLA-DQ and -DP as a restriction element. Int J Cancer, 2008. 122(3): p. 486-94.
17. Evans, E. M., et al., Infiltration of cervical cancer tissue with human papillomavirus-specific cytotoxic T-lymphocytes. Cancer Res, 1997. 57(14): p. 2943-50.
18. Triozzi, P. L. and A. P. Fernandez, The role of the immune response in merkel cell carcinoma. Cancers (Basel), 2013. 5(1): p. 234-54.
19. Simoni, Y., et al., Bystander CD8(+) T cells are abundant and phenotypically distinct in human tumour infiltrates. Nature, 2018. 557(7706): p. 575-579.
20. Rosato, P. C., et al., Virus-specific memory T cells populate tumors and can be repurposed for tumor immunotherapy. Nat Commun, 2019. 10(1): p. 567.
21. Scheper, W., et al., Low and variable tumor reactivity of the intratumoral TCR repertoire in human cancers. Nat Med, 2019. 25(1): p. 89-94.
22. Yu, W., et al., Clonal Deletion Prunes but Does Not Eliminate Self-Specific alphabeta CD8(+) T Lymphocytes. Immunity, 2015. 42(5): p. 929-41.
23. Glanville, J., et al., Identifying specificity groups in the T cell receptor repertoire. Nature, 2017. 547(7661): p. 94-98.
24. Huang, H., et al., Analyzing the CD4+ T cell response repertoire to M. tuberculosis using GLIPH2 and whole-genome antigen screening. Nat Biotechnol, 2020.
25. Reuben, A., et al., Comprehensive T cell repertoire characterization of non-small cell lung cancer. Nat Commun, 2020. 11(1): p. 603.
26. Joshi, K., et al., Spatial heterogeneity of the T cell receptor repertoire reflects the mutational landscape in lung cancer. Nat Med, 2019. 25(10): p. 1549-1559.
27. Shugay, M., et al., VDJdb: a curated database of T-cell receptor sequences with known antigen specificity. Nucleic Acids Res, 2018. 46(D1): p. D419-D427.
28. Song, I., et al., Broad TCR repertoire and diverse structural solutions for recognition of an immunodominant CD8(+) T cell epitope. Nat Struct Mol Biol, 2017. 24(4): p. 395-406.
29. Sidney, J., et al., HLA class I supertypes: a revised and updated classification. BMC Immunol, 2008. 9: p. 1.
30. Harjanto, S., L. F. Ng, and J. C. Tong, Clustering HLA class I superfamilies using structural interaction patterns. PLoS One, 2014. 9(1): p. e86655.
31. Robins, H. S., et al., Overlap and effective size of the human CD8+ T cell receptor repertoire. Sci Transl Med, 2010. 2(47): p. 47ra64.
32. Arstila, T. P., et al., A direct estimate of the human alphabeta T cell receptor diversity. Science, 1999. 286(5441): p. 958-61.
33. Gee, M. H., et al., Antigen Identification for Orphan T Cell Receptors Expressed on Tumor-Infiltrating Lymphocytes. Cell, 2018. 172(3): p. 549-563 e16.
34. Kheir, F., et al., Detection of Epstein-Barr Virus Infection in Non-Small Cell Lung Cancer. Cancers (Basel), 2019. 11(6).
35. Han, A., et al., Linking T-cell receptor sequence to functional phenotype at the single-cell level. Nat Biotechnol, 2014. 32(7): p. 684-92.
36. Stubbington, M. J. T., et al., T cell fate and clonality inference from single-cell transcriptomes. Nat Methods, 2016. 13(4): p. 329-332.
37. Guo, X., et al., Global characterization of T cells in non-small-cell lung cancer by single-cell sequencing. Nat Med, 2018. 24(7): p. 978-985.
38. Joglekar, A. V., et al., T cell antigen discovery via signaling and antigen-presenting bifunctional receptors. Nat Methods, 2019. 16(2): p. 191-198.
39. Li, G., et al., T cell antigen discovery via trogocytosis. Nat Methods, 2019. 16(2): p. 183-190.
40. Kula, T., et al., T-Scan: A Genome-wide Method for the Systematic Discovery of T Cell Epitopes. Cell, 2019. 178(4): p. 1016-1028 e13.
41. Sewell, A. K., Why must T cells be cross-reactive? Nat Rev Immunol, 2012. 12(9): p. 669-77.
42. McCarthy, E. F., The toxins of William B. Coley and the treatment of bone and soft-tissue sarcomas. Iowa Orthop J, 2006. 26: p. 154-8.
43. Morales, A., D. Eidinger, and A. W. Bruce, Intracavitary Bacillus Calmette-Guerin in the treatment of superficial bladder tumors. J Urol, 1976. 116(2): p. 180-3.
44. Gopalakrishnan, V., et al., Gut microbiome modulates response to anti-PD-1 immunotherapy in melanoma patients. Science, 2018. 359(6371): p. 97-103.
45. Routy, B., et al., Gut microbiome influences efficacy of PD-1-based immunotherapy against epithelial tumors. Science, 2018. 359(6371): p. 91-97.
46. Vetizou, M., et al., Anticancer immunotherapy by CTLA-4 blockade relies on the gut microbiota. Science, 2015. 350(6264): p. 1079-84.
47. Sivan, A., et al., Commensal Bifidobacterium promotes antitumor immunity and facilitates anti-PD-L1 efficacy. Science, 2015. 350(6264): p. 1084-9.
48. Matson, V., et al., The commensal microbiome is associated with anti-PD-1 efficacy in metastatic melanoma patients. Science, 2018. 359(6371): p. 104-108.
49. Riquelme, E., et al., Tumor Microbiome Diversity and Composition Influence Pancreatic Cancer Outcomes. Cell, 2019. 178(4): p. 795-806 e12.
50. Emerson, R. O., et al., Immunosequencing identifies signatures of cytomegalovirus exposure history and HLA-mediated effects on the T cell repertoire. Nat Genet, 2017. 49(5): p. 659-665.
51. Abazeed, M. E., et al., Integrative radiogenomic profiling of squamous cell lung cancer. Cancer Res, 2013. 73(20): p. 6289-98.
52. Barbie, D. A., et al., Systematic RNA interference reveals that oncogenic KRAS-driven cancers require TBKI. Nature, 2009. 462(7269): p. 108-12.
53. Altman, J. D. and M. M. Davis, MHC-peptide tetramers to visualize antigen-specific T cells. Curr Protoc Immunol, 2003. Chapter 17: p. Unit 17 3.
54. Picelli, S., et al., Full-length RNA-seq from single cells using Smart-seq2. Nat Protoc, 2014. 9(1): p. 171-81.
55. Rueden, C. T., et al., ImageJ2: ImageJ for the next generation of scientific image data. BMC Bioinformatics, 2017. 18(1): p. 529.
56. Schindelin, J., et al., Fiji: an open-source platform for biological-image analysis. Nat Methods, 2012. 9(7): p. 676-82.
57. Hellmann, M. D., et al., Circulating tumor DNA analysis to assess risk of progression after long-term response to PD-(L)1 blockade in NSCLC. Clin Cancer Res, 2020.
58. Newman, A. M., et al., An ultrasensitive method for quantitating circulating tumor DNA with broad patient coverage. Nat Med, 2014. 20(5): p. 548-54.
59. Koboldt, D. C., et al., VarScan 2: somatic mutation and copy number alteration discovery in cancer by exome sequencing. Genome Res, 2012. 22(3): p. 568-76.
60. Cibulskis, K., et al., Sensitive detection of somatic point mutations in impure and heterogeneous cancer samples. Nat Biotechnol, 2013. 31(3): p. 213-9.
61. Saunders, C. T., et al., Strelka: accurate somatic small-variant calling from sequenced tumor-normal sample pairs. Bioinformatics, 2012. 28(14): p. 1811-7.
62. Lek, M., et al., Analysis of protein-coding genetic variation in 60,706 humans. Nature, 2016. 536(7616): p. 285-91.

What is claimed is:

1. An construct comprising at least one complementary determining region (CDR) having at least 70%, 75%, 80%, 85%, 90%, 95%, 96%, 97%, 98%, 99%, or 100% sequence identity to a sequence selected from the group consisting of SEQ ID NOs: 1-14, 26-33, and 48-49.

2. The construct of claim 1, wherein the construct is capable of binding to an epitope comprising a sequence having at least 70%, 75%, 80%, 85%, 90%, 95%, 96%, 97%, 98%, 99%, or 100% sequence identity to a sequence selected from the group consisting of SEQ ID NOs: 15-22 and 44-45.

3. The construct of any one of claims 1 to 2, wherein the construct is a single-chain construct or a double-chain construct.

4. The construct of any one of claims 1 to 3, wherein the construct is selected from the group consisting of: (a) a T cell receptor (TCR); (b) an antibody; and (c) a functional derivative or fragment of (a) or (b).

5. The construct of any one of claims 1 to 4, wherein the construct is a TCR construct comprising a TCR alpha chain and a TCR beta chain covalently linked to each other.

6. The construct of any one of claims 1 to 5, wherein the construct is a TCR construct comprising in its beta chain a CDR3β having at least 70%, 75%, 80%, 85%, 90%, 95%, 96%, 97%, 98%, 99%, or 100% sequence identity to a sequence selected from SEQ ID NOs: 1-14, 26-33, and 48-49.

7. The construct of claim 6, wherein the construct further comprising in its alpha chain a CDR3α sequence.

8. The construct of claim 7, wherein the CDR3α sequence has at least 70%, 75%, 80%, 85%, 90%, 95%, 96%, 97%, 98%, 99%, or 100% sequence identity to the sequence of SEQ ID NO: 24.

9. The construct of any one of claims 1 to 4, wherein construct is an antibody construct selected from the group consisting of an antigen-binding fragment (Fab), a single-chain variable fragment (scFv), a nanobody, a single domain antibody (sdAb), a V_{H} domain, a V_{L} domain, a V_{H}H domain, a diabody, or a functional fragment of any thereof.

10. A recombinant nucleic acid comprising a nucleic acid sequence encoding a construct according to any one of claims 1 to 8.

11. The nucleic acid of claim 10, wherein the nucleic acid sequence is operably linked to a heterologous nucleic acid sequence.

12. The nucleic acid of any one of claims 10 to 11, wherein the nucleic acid molecule is further defined as an expression cassette or a vector.

13. The nucleic acid of claim 12, wherein the vector is a plasmid vector or a viral vector.

14. The nucleic acid of claim 13, wherein the viral vector is derived from a lentivirus, an adeno virus, an adeno-associated virus, a baculovirus, or a retrovirus.

15. An engineered cell comprising:
a) a construct according to any one of claims 1 to 9; and/or
b) a recombinant nucleic acid according to any one of claims 10 to 14.

16. The engineered cell of claim 15, wherein the engineered cell is a eukaryotic cell.

17. The engineered cell of claim 16, wherein the eukaryotic cell is a mammalian cell.

18. The engineered cell of claim 17, wherein the mammalian cell is a human cell.

19. The engineered cell of any one of claims 15 to 18, wherein the cell is an immune cell.

20. The engineered cell of claim 19, wherein the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

21. The engineered cell of claim 20, wherein the immune cell is a lymphocyte.

22. The engineered cell of claim 21, wherein the lymphocyte is a T lymphocyte or a T lymphocyte progenitor.

23. The engineered cell of claim 22, wherein the T lymphocyte is a CD4+ T cell or a CD8+ T cell.

24. The engineered cell of any one of claims 22 to 23, wherein the T lymphocyte is a CD8+ T cytotoxic lymphocyte cell selected from the group consisting of naïve CD8+ T cells, central memory CD8+ T cells, effector memory CD8+ T cells, effector CD8+ T cells, CD8+ stem memory T cells, and bulk CD8+ T cells.

25. The engineered cell of any one of claims 22 to 23, wherein the T lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

26. A method for making an engineered cell, comprising:
a) providing a host cell capable of protein expression; and
b) transducing the provided host cell with a recombinant nucleic acid according to any one of claims 10 to 14 to produce an engineered cell.

27. An engineered cell produced by a method according to claim 26.

28. A cell culture comprising at least one engineered cell of any one of claims 15-25 and 27, and a culture medium.

29. A pharmaceutical composition comprising a pharmaceutically acceptable carrier and:
a) a construct according to any one of claims 1 to 9;
b) a recombinant nucleic acid according to any one of claims 10 to 14; and/or
c) an engineered cell according to any one of claims 15-25 and 27.

30. The pharmaceutical composition of claim 29, wherein the composition comprises a recombinant nucleic acid according to any one of claims 10 to 14, and a pharmaceutically acceptable carrier.

31. The pharmaceutical composition of claim 30, wherein the recombinant nucleic acid is encapsulated in a viral capsid or a lipid nanoparticle.

32. The pharmaceutical composition of claim 29, wherein the composition comprises an engineered cell according to any one of claims 15-25 and 27.

33. A method for the prevention and/or treatment of a condition in a subject in need thereof, the method comprising administering to the subject a composition comprising:
a) a construct according to any one of claims 1 to 9;
b) a recombinant nucleic acid according to any one of claims 10 to 14;
c) an engineered cell according to any one of claims 15-25 and 27; and/or
d) a pharmaceutically composition according to any one of claims 29 to 32.

34. The method of claim 33, wherein the condition is associated with a proliferative disorder.

35. The method of claim 34, wherein the proliferative disorder is a cancer that expresses an epitope comprising a sequence with at least 70%, 75%, 80%, 85%, 90%, 95%, 96%, 97%, 98%, 99%, or 100% sequence identity to a sequence selected from the group consisting of SEQ ID NOs: 15-22 and 44-45.

36. The method of any one of claims 34 to 35, wherein the proliferative disorder is a cancer expressing the TMEM161A antigen (TMEM161A-positive cancer).

37. The method of claim 36, wherein the TMEM161A-positive cancer is selected from the group consisting of colon cancer, breast cancer, kidney cancer, lung cancer, ovarian cancer, pancreatic cancer, prostate cancer, sarcoma, neuroendocrine cancer, and testicular cancer.

38. The method of any one of claims 34 to 35, wherein the proliferative disorder is a cancer expressing the CLDN2 antigen (CLDN2-positive cancer).

39. The method of claim 38, wherein the CLDN2-positive cancer is selected from the group consisting of colorectal cancer, cervical cancer, liver cancer, lung cancer, gastric cancer, pancreatic cancer, renal cancer, and stomach cancer.

40. The method of claim 37 or 39, wherein the lung cancer is selected from the group consisting of adenocarcinoma, squamous cell carcinoma, small cell carcinoma, non-small cell carcinoma, adenosquamous carcinoma, small cell lung cancer, large cell carcinoma, neuroendocrine cancers of the lung, non-small cell lung cancer (NSCLC), undifferentiated non-small cell carcinoma, non-small cell carcinoma not otherwise specified, pulmonary squamous cell carcinoma, broncho-alveolar carcinoma, sarcomatoid carcinoma, pleomorphic carcinoma, carcinosarcoma, pulmonary blastoma, metastatic carcinoma of unknown primary, primary pulmonary lymphoepithelioma-like carcinoma, and benign neoplasms of the lung.

41. The method of any one of claims 33 to 40, wherein the cancer is a non-metastatic cancer, a metastatic cancer, a multiply drug resistant cancer, or a recurrent cancer.

42. The method of claim 41, wherein the administered composition inhibits tumor growth or metastasis of the cancer in the subject.

43. The method of claim 42, wherein the condition is a malignancy associated with a bacterial infection or viral infection.

44. The method of claim 43, wherein the condition is a malignancy associated with an infection by Epstein-Barr virus (EBV) or Escherichia coli.

45. The method of claim 44, wherein the malignancy is associated with an EBV infection and is selected from the group consisting of Hodgkin lymphoma, Burkitt lymphoma, diffuse large B cell lymphoma, nasopharyngeal carcinoma, gastric carcinoma, post-transplant lymphoproliferative disease, B lymphoproliferative disease, T/NK lymphoproliferative disease, T/NK lymphomas/leukemias, leiomyosarcomas, and lymphoepithelioma-like carcinomas.

46. The method of any one of claims 33 to 45, wherein the subject is a mammal.

47. The method of claim 46, wherein the mammal is a human.

48. The method of any one of claims 33 to 47, wherein the composition is administered to the subject individually as a first therapy or in combination with at least one additional therapies.

49. The method of claim 48, wherein the at least one additional therapies is selected from the group consisting of chemotherapy, radiotherapy, immunotherapy, hormonal therapy, toxin therapy, targeted therapy, and surgery.

50. The method of any one of claims 48 to 49, wherein the first therapy and the at least one additional therapies are administered concomitantly.

51. The method of any one of claims 48 to 50, wherein the first therapy is administered at the same time as the at least one additional therapies.

52. The method of any one of claims 48 to 50, wherein the first therapy and the at least one additional therapies are administered sequentially.

53. The method of claim 52, wherein the first therapy is administered before the at least one additional therapies.

54. The method of claim 52, wherein the first therapy is administered after the at least one additional therapies.

55. The method of claim 52, wherein the first therapy is administered before and/or after the at least one additional therapies.

56. The method of any one of claims 48 to 55, wherein the first therapy and the at least one additional therapies are administered in rotation.

57. The method of any one of claims 48 to 49, wherein the first therapy and the at least one additional therapies are administered together in a single formulation.

58. A kit for the diagnosis, prevention, and/or treatment a condition in a subject in need thereof, the system comprising:
a) a construct according to any one of claims 1 to 9;
b) a recombinant nucleic acid according to any one of claims 10 to 14;
c) an engineered cell according to any one of claims 15-25 and 27; and/or
d) a pharmaceutically composition according to any one of claims 29 to 32.

59. A method for obtaining a construct according to claim 1, the method comprising:
a) identifying a plurality of T cell receptors (TCRs) associated with a health condition;
b) determining a sequence of a CDR3β present in each of the identified TCRs;
c) identifying one or more cognate antigens commonly recognized by the CDR3β sequences;
c) making a construct comprising a CDR3β sequence determined in (b), wherein the construct is capable of binding to the one or more cognate antigens.
